(19) United States
(12) Patent Application Publication
(10) Pub. No.: US 2023/0176070 A1
(43) Pub. Date: Jun. 8, 2023

(54) COMPOSITIONS AND METHODS FOR IDENTIFYING NANOBODIES AND NANOBODY AFFINITIES

(71) Applicant: UNIVERSITY OF PITTSBURGH-OF THE COMMONWEALTH SYSTEM OF HIGHER EDUCATION, Pittsburgh, PA (US)

(72) Inventors: Yi SHI, Wexford, PA (US); Yufei XIANG, Pittsburgh, PA (US); Zhe SANG, Pittsburgh, PA (US)

(21) Appl. No.: 17/922,685

(22) PCT Filed: Apr. 29, 2021
(86) PCT No.: PCT/US21/29869

§ 371 Date: Nov. 1, 2022

Related U.S. Application Data
(60) Provisional application No. 63/018,559, filed on May 1, 2020.

(51) Int. Cl. G01N 33/68; G16B 35/10; G16B 35/20

(52) U.S. Cl. CPC G01N 33/6857; G01N 33/6848; G16B 35/10; G16B 35/20

(57) ABSTRACT

Provided herein are methods of identifying a group of complementarity determining region (CDR)3, 2 and/or 1 nanobody amino acid sequences (CDR3, CDR2 and/or CDR1 sequences) wherein a reduced number of the CDR3, CDR2 and/or CDR1 sequences are false positives as compared to a control, methods for determining antigen affinity of nanobody peptide sequences, and related methods for training a deep learning model.

### CROSS-REFERENCE TO RELATED APPLICATIONS

This application claims the benefit of U.S. Provisional Application No. 63/018,559, filed May 1, 2020, which is expressly incorporated herein by reference in its entirety.

### BACKGROUND

Nanobodies (Nbs) are natural antigen-binding fragments derived from the VHH domain of camelid heavy-chain only antibodies (HcAbs). They are characterized by their small size and outstanding structural robustness, excellent solubility and stability, ease of bioengineering and manufacturing, low immunogenicity in humans and fast tissue penetration. For these reasons, Nbs have emerged as promising agents for cutting-edge biomedical, diagnostic and therapeutic applications (Muyldermans, 2013; Beghein, 2017; Rasmussen, 2011; Jovcevska, I. & Muyldermans, S, 2020).

Display-based technologies have been developed for Nb discovery (Lauwereys, 1998; Pardon, 2014; McMahon, 2018; Egloff, 2019). These methods usually yield a small handful of target synthetic Nbs that bind specific targets with moderate affinities and do not directly analyze naturally circulating, antigen-specific HcAb/Nb repertoires. Recently, mass spectrometry-based proteomics has emerged as a promising technique for Nb discovery (Fridy, 2014). However, significant challenges remain towards a large-scale, sensitive, and reliable analysis of antigen-specific Nb proteomes for at least several reasons: (a) the diversity and dynamic range of circulating antibodies are orders of magnitude higher than any cellular proteome. (b) A Nb sequence database, obtained from an immunized camelid, usually contains millions of unique sequences posing a challenge for accurate database search (Savitski, 2015). (c) This massive database is overrepresented by conserved Nb framework sequences, which provide little specificity for identification. The specificity is largely determined by complementarity-determining regions (CDRs), among which CDR3 loops can be long, rendering it difficult for confident MS analysis. (d) Current methods are limited by the availability of efficient protocols and informatics that enable accurate quantification and classification of large Nb repertoires.

### SUMMARY

Provided herein is a method of identifying a group of complementarity determining region (CDR)3, 2, and/or 1 nanobody amino acid sequences (CDR3, CDR2 and/or CDR1 sequences) wherein a reduced number of the CDR3, CDR2 and/or CDR1 sequences are false positives as compared to a control, the method comprising: (a) obtaining a blood sample from a camelid immunized with an antigen; (b) using the blood sample to obtain a nanobody cDNA library; (c) identifying the sequence of each cDNA in the library; (d) isolating nanobodies from the same or a second blood sample from the camelid immunized with the antigen; (e) digesting the nanobodies with trypsin or chymotrypsin to create a group of digestion products; (f) performing a mass spectrometry analysis of the digestion products to obtain mass spectrometry data; (g) selecting sequences identified in step c. that correlate with the mass spectrometry data; (h) identifying sequences of CDR3, CDR2 and/or CDR1 regions in the sequences from step g.; and (i) selecting from the CDR3, CDR2 and/or CDR1 region sequences of step h. those sequences having equal to or more than a required fragmentation coverage percentage; wherein the selected sequences of step (i) comprise a group having the reduced number of false positive CDR3, CDR2 and/or CDR1 sequences. In some embodiments, step (d) comprises obtaining plasma from the blood sample and isolating nanobodies using one or more affinity isolation methods. In some aspects, the one or more affinity isolation methods of step (d) comprise one or more of protein G sepharose affinity chromatography and protein A sepharose affinity chromatography. In some aspects, step (d) further comprises a functional selection step comprising selecting antigen-specific nanobodies using an antigen-specific affinity chromatography and eluting the antigen-specific nanobodies under varying degrees of stringency thereby creating different nanobody fractions, and performing steps (e) through (i) on each fraction individually and estimating an affinity of each different step (i) CDR3, CDR2 and/or CDR1 region sequence for the antigen based on a relative abundance of the CDR3, CDR2 and/or CDR1 region sequence, respectively, in each of the nanobody fractions.

In some embodiments, a group of complementarity determining region (CDR)3 nanobody amino acid sequences (CDR2 sequences) wherein a reduced number of the CDR3 sequences are false positives as compared to a control, the method comprising: (a) obtaining a blood sample from a camelid immunized with an antigen; (b) using the blood sample to obtain a nanobody cDNA library; (c) identifying the sequence of each cDNA in the library; (d) isolating nanobodies from the same or a second blood sample from the camelid immunized with the antigen; (e) digesting the nanobodies with trypsin or chymotrypsin to create a group of digestion products; (f) performing a mass spectrometry analysis of the digestion products to obtain mass spectrometry data; (g) selecting sequences identified in step c. that correlate with the mass spectrometry data; (h) identifying sequences of CDR3 regions in the sequences from step g.; and (i) selecting from the CDR3 region sequences of step h. those sequences having equal to or more than a required fragmentation coverage percentage; wherein the selected sequences of step (i) comprise a group having the reduced number of false positive CDR3 sequences. In some embodiments, step (d) comprises obtaining plasma from the blood sample and isolating nanobodies using one or more affinity isolation methods. In some aspects, the one or more affinity isolation methods of step (d) comprise one or more of protein G sepharose affinity chromatography and protein A sepharose affinity chromatography. In some aspects, step (d) further comprises a functional selection step comprising selecting antigen-specific nanobodies using an antigen-specific affinity chromatography and eluting the antigen-specific nanobodies under varying degrees of stringency thereby creating different nanobody fractions, and performing steps (e) through (i) on each fraction individually and estimating an affinity of each different step (i) CDR3 region sequence for the antigen based on a relative abundance of the CDR3 region sequence in each of the nanobody fractions.

In some embodiments, a group of complementarity determining region (CDR)2 nanobody amino acid sequences (CDR2 sequences) wherein a reduced number of the CDR2 sequences are false positives as compared to a control, the method comprising: (a) obtaining a blood sample from a camelid immunized with an antigen; (b) using the blood sample to obtain a nanobody cDNA library; (c) identifying the sequence of each cDNA in the library; (d) isolating nanobodies from the same or a second blood sample from the camelid immunized with the antigen; (e) digesting the nanobodies with trypsin or chymotrypsin to create a group of digestion products; (f) performing a mass spectrometry analysis of the digestion products to obtain mass spectrometry data; (g) selecting sequences identified in step c. that correlate with the mass spectrometry data; (h) identifying sequences of CDR2 regions in the sequences from step g.; and (i) selecting from the CDR2 region sequences of step h. those sequences having equal to or more than a required fragmentation coverage percentage; wherein the selected sequences of step (i) comprise a group having the reduced number of false positive CDR2 sequences. In some embodiments, step (d) comprises obtaining plasma from the blood sample and isolating nanobodies using one or more affinity isolation methods. In some aspects, the one or more affinity isolation methods of step (d) comprise one or more of protein G sepharose affinity chromatography and protein A sepharose affinity chromatography. In some aspects, step (d) further comprises a functional selection step comprising selecting antigen-specific nanobodies using an antigen-specific affinity chromatography and eluting the antigen-specific nanobodies under varying degrees of stringency thereby creating different nanobody fractions, and performing steps (e) through (i) on each fraction individually and estimating an affinity of each different step (i) CDR2 region sequence for the antigen based on a relative abundance of the CDR2 region sequence in each of the nanobody fractions.

In some embodiments, a group of complementarity determining region (CDR)1 nanobody amino acid sequences (CDR1 sequences) wherein a reduced number of the CDR1 sequences are false positives as compared to a control, the method comprising: (a) obtaining a blood sample from a camelid immunized with an antigen; (b) using the blood sample to obtain a nanobody cDNA library; (c) identifying the sequence of each cDNA in the library; (d) isolating nanobodies from the same or a second blood sample from the camelid immunized with the antigen; (e) digesting the nanobodies with trypsin or chymotrypsin to create a group of digestion products; (f) performing a mass spectrometry analysis of the digestion products to obtain mass spectrometry data; (g) selecting sequences identified in step c. that correlate with the mass spectrometry data; (h) identifying sequences of CDR1 regions in the sequences from step g.; and (i) selecting from the CDR1 region sequences of step h. those sequences having equal to or more than a required fragmentation coverage percentage; wherein the selected sequences of step (i) comprise a group having the reduced number of false positive CDR1 sequences. In some embodiments, step (d) comprises obtaining plasma from the blood sample and isolating nanobodies using one or more affinity isolation methods. In some aspects, the one or more affinity isolation methods of step (d) comprise one or more of protein G sepharose affinity chromatography and protein A sepharose affinity chromatography. In some aspects, step (d) further comprises a functional selection step comprising selecting antigen-specific nanobodies using an antigen-specific affinity chromatography and eluting the antigen-specific nanobodies under varying degrees of stringency thereby creating different nanobody fractions, and performing steps (e) through (i) on each fraction individually and estimating an affinity of each different step (i) CDR1 region sequence for the antigen based on a relative abundance of the CDR1 region sequence in each of the nanobody fractions.

In some embodiments, the antigen-specific affinity chromatography is a resin conjugated to the antigen. In some embodiments, the antigen-specific affinity chromatography is a resin coupled to a protein tag and the antigen. In some embodiments, the antigen-specific affinity chromatography is a resin coupled to a maltose binding protein and the antigen.

Some aspects further comprise creating a CDR3, CDR2, or CDR1 peptide having a sequence identified in step (i). Some aspects further comprise creating a nanobody comprising a CDR3, CDR2, and/or CDR1 region having a sequence identified in step (i).

Also included herein is a nanobody comprising an amino acid sequence selected from SEQ ID NOs: 1-2536 and SEQ ID NOs: 2665-2667.

Further provided herein is a computer-implemented method, comprising: (a) receiving a nanobody peptide sequence; (b) identifying a plurality of complementarity-determining region (CDR) regions of the nanobody peptide sequence, the CDR regions including CDR3, CDR2 and/or CDR1 regions; (c) applying a fragmentation filter to discard one or more false positive CDR3, CDR2 and/or CDR1 regions of the nanobody peptide sequence; (d) quantifying an abundance of one or more non-discarded CDR3, CDR2 and/or CDR1 regions of the nanobody peptide sequence; and (e) inferring an antigen affinity based on the quantified abundance of the one or more non-discarded CDR3, CDR2 and/or CDR1 regions of the nanobody peptide sequence.

In some embodiments, the computer-implemented method further comprises classifying the one or more non-discarded CDR3, CDR2 and/or CDR1 regions of the nanobody peptide sequence as having a low antigen affinity, mediocre antigen affinity, or high antigen affinity.

In some embodiments, the computer-implemented method further comprises assembling the one or more non-discarded CDR3, CDR2 and/or CDR1 regions of the nanobody peptide sequence classified as having the high antigen affinity into a nanobody protein.

In some aspects of the computer-implemented method, the fragmentation filter is configured to require a minimum calculated fragmentation coverage percentage. In other or further aspects, the minimum calculated fragmentation coverage percentage is about 30. In some aspects, the minimum calculated fragmentation coverage percentage is about 50 for trypsin-treated samples and about 40 for chymotrypsin-treated samples.

In some embodiments, the computer-implemented method further comprises receiving a plurality of nanobody peptide sequences; and comparing each of the nanobody peptide sequences to a database to separate the nanobody peptide sequences into an excluded subgroup and a non-excluded subgroup, wherein the nanobody peptide sequences of the excluded subgroup are not found in the database, and wherein the CDR regions are only identified in the nanobody peptide sequences of the non-excluded subgroup.

In some embodiments of the computer-implemented method, the abundance of one or more non-discarded CDR3, CDR2 and/or CDR1 regions of the nanobody peptide sequence is quantified based on relative MS1 ion signal intensities. In some embodiments, the antigen affinity is inferred using k-means clustering based on epitope similarity.

Also provided herein is a method for training a deep learning model, comprising: creating a dataset using the computer-implemented method described above; and training, using the dataset, a deep learning model to classify nanobody peptide sequences having low antigen affinity and nanobody peptide sequences having high antigen affinity, wherein the dataset comprises a plurality of nanobody peptide sequences and corresponding antigen-affinity labels. In some embodiments, the deep learning model is a convolutional neural network.

Further provided herein is a method for determining antigen affinity of nanobody peptide sequences, comprising: receiving a nanobody peptide sequence; inputting the nanobody peptide sequence into a trained deep learning model; and classifying, using the trained deep learning model, the nanobody peptide sequence as having low antigen affinity or high antigen affinity. In some embodiments, the deep learning model is a convolutional neural network. In some embodiments, the trained deep learning model is trained according to method for training a deep learning model described above

### DESCRIPTION OF DRAWINGS

FIG. 1(A-K). In-silico analysis of a NGS Nb database reveals the superiority of chymotrypsin for Nb proteomics. (A) A Nb crystal structure (PDB: 4QGY). CDR loops are color coded. (B) Sequence length distributions of CDRs of the database. (C) In-silico digestion of the Nb database by two proteases and a cumulative plot of corresponding peptide masses. (D) The length distributions for both trypsin and chymotrypsin digested CDR3 peptides. (E) Complementarity of trypsin and chymotrypsin for Nb mapping based on simulation. 10,000 Nbs with unique CDR3 sequences were randomly selected and in silico digested to produce CDR3 peptides. Peptides with molecular weights of 0.8-3 kDa and with sufficient CDR3 coverage (>30%) were used for Nb mapping. (F-G) Evaluations of unique CDR3 peptide identifications (1F: trypsin; 1G: chymotrypsin) based on the percentage of CDR3 fragment ions that were matched in the MS/MS spectra. CDR3 peptides were identified by database search using either the “target” database (in salmon) or the “decoy” database (in grey). (H-K) 3D plots of the normalized CDR3 peptide identifications from the target database search, the percentages of CDR3 fragmentations, and CDR3 length. FDR: false discovery rate. FDRs of CDR3 identifications are colored on the 3D plots. The color bar shows the scale of FDR. FDR below 5% are presented in gradient red. (1H: analysis by trypsin; 1I: analysis by chymotrypsin.) (J-L). Representative high-quality MS/MS spectra of trypsin and chymotrypsin-digested CDR3 peptides. The sequence in FIG. 1K is NTVYLEMNSLKPEDTAVYSCAAGVSDYGCYR (SEQ ID NO: 2656). The sequence in FIG. 1L is YCAAAEGLASGSY (SEQ ID NO: 2657).

FIG. 2(A-G). Schematics of the hybrid proteomic pipeline for reliable and in-depth analysis of antigen-engaged Nb proteomes. (A) Schematic of the pipeline for Nb proteomics. The pipeline consists of three main components: camelid immunization and purification of antigen-specific Nbs, proteomic analysis of Nbs (facilitated by a dedicated software Augur Llama and deep-learning), and high-throughput integrative structural analysis of antigen-Nb complexes. (B) ELISA measurements of the camelid immune responses of three antigens of GST, HSA and the PDZ. (C) Identifications of unique CDR combinations and unique CDR3 sequences for different antigens. (D) A comparison between trypsin and chymotrypsin for CDR3 mapping of high-quality Nb_{GST}. (E) Comparisons of Nb_{GST} CDR3 identifications by three different proteases (gluC, trypsin and chymotrypsin). The results were based on three independent experiments. (F) The solubility of the randomly selected antigen-specific Nbs. (G) Verifications of the selected Nbs for antigen binding.

FIG. 3(A-L). Classification of Nb repertoires for GST, HSA and PDZ binding. (A) Label-free MS quantification and heat map analysis of CDR3_{GST} fingerprints by chymotrypsin. (B) Reproducibility and precision of label-free CDR3_{GST} peptide quantifications by chymotrypsin. (C) Percentages of different Nb affinity clusters that were classified by quantitative proteomics. (D) Linear Correlation (R²=0.85) of Nb ELISA affinities (LogIC50 of O.D. 450 nm) with SPR K_{D} measurements. (E) Boxplots of ELISA affinities of different Nb clusters. The p values were calculated based on the student's t test. * indicates a p value of <0.05, ** indicates <0.01, *** indicates <0.001, **** indicates <0.0001, ns indicates not significant. (F) A plot summarizing ELISA affinities of 25 Nb_{HSA} (circles), O.D. at 450 nm. K_{D} affinities of the top 14 ranked Nbs by ELISA were measured by SPR (triangles). (G) A plot summarizing the ELISA affinities of 11 soluble Nb_{PDZ}. (H) SPR kinetics analysis of representative Nb_{GST} from three different affinity clusters. For G60(C1), Ka(1/Ms)=4.9e3, Kd(1/s)=5.9e-3, K_{D}=1.3 μM; for G95(C2), Ka(1/Ms)=1.4e4, Kd(1/s)=1.1e-3, K_{D}=77 nM; For G13(C3), Ka(1/Ms)=4.74e5, Kd(1/s)=1.7e-4, K_{D}=360 pM. (I) A representative SPR kinetics measurements of high-affinity Nb_{HSA}. For H14, Ka(1/Ms)=2.5e5, Kd(1/s)=5.75e-6, K_{D}=22.3 pM. (J) The SPR kinetics measurement of Nb_{PDZ} P10. For P10, Ka(1/Ms)=2.06e6, Kd(1/s)=9.03e-6, K_{D}=4.4 pM. (K) Immunoprecipitations of GST (1 nM) by different Nbs-coupled dynabeads and GSH resin. (L) Schematic of the PDZ domain of the mammalian mitochondrial outer membrane protein 25. Fluorescence microscopic analysis of Nb_{PDZ} P10. The Nb was conjugated by Alexa Fluor 647 for native mitochondrial immunostaining of the COS-7 cell line. Mitotracker was used for positive control.

FIG. 4(A-K). The structural landscapes of HSA-specific Nb proteomes revealed by the integrative structural methods. (A) The sequence variations of pI and hydropathy between human and camelid serum albumins (upper panel,). The heatmap of the major epitopes mapped by structural docking (lower panel). (B) Cartoon representations of the four dominant HSA epitopes. HSA are presented in gray. E1, E2 and E3 are in salmon, orange and cyan, respectively. (C) Surface representations showing co-localizations of electrostatic potential surfaces with three major epitopes. (D) The HSA epitopes and their fractions (%) based on converged cross-link models (E1: residues 57-62, 135-169; E2: 322-331, 335, 356-365, 395-410; E3: 29-37, 86-91, 117-123, 252-290; E4: 566-585, 595, 598-606 and E5:188-208, 300-306, 463-468). (E-G) Representative cross-link models of HSA-Nb complexes. The best scoring models were presented. Satisfied DSS or EDC cross-links are shown as blue sticks. (H) A putative salt bridge between glutamic acid 400 (HSA) and arginine 108 of a Nb CDR3 is presented. The local sequence alignment between HSA and camelid albumin is shown. (I) ELISA affinity screening (heatmap) of 19 different Nbs for binding to wild type HSA and the point mutant (E400R). * indicates decreased affinity. (J) A plot of the RMSDs (room-median-square-deviations) of HSA-Nb cross-link models. (K) Bar plots showing the percentage of all the DSS and EDC cross-links of HSA-Nbs that satisfied the models.

FIG. 5(A-K). Mechanisms of Nb affinity maturation. (A) Distributions of CDR3 lengths of high-affinity (dark) and low-affinity (light) Nb_{GST} and Nb_{HSA} (B) Comparisons of the pI of different Nbs. (C-D) Comparisons of pI and hydropathy of CDRs between different Nbs. (E) A plot of CDR3 sequences. The alignment is based on a random selection of 1,000 unique CDR3 sequences with the identical length of 15 residues. Schematic of CDR3 architecture: the hypervariable “head” is in dark grey and the semi-variable “torso” is in pale grey. (F) Pie charts of the amino acid compositions of the CDR3 heads (Nb_{GST} and Nb_{HSA}) and the CDR2s (Nb_{GST}) Only the top 6 abundant residues are shown. (G) The relative changes of abundant amino acids on CDR3 heads of both Nb_{GST} and Nb_{HSA}. Positive charged residues of K(lysine)/R(arginine)/H(histidine), negative charged residues of D(aspartic acid)/E(glutamic acid), aromatic residue of Y(tyrosine) and small flexible amino acids of G(glycine)/S(serine) are shown. (H) Comparisons of the relative abundance of Y, G and S on the CDR3 heads between high-affinity and low-affinity Nb_{HSA}. Their relative abundances are plotted as a function of the relative position of the respective residues. A representative structure (PDB: 5F1O) of antigen-Nb complex showing two tyrosines on the CDR3 head are inserted into the deep pockets of the antigen. (I) Correlation plots of the ELISA affinities and the number of specific amino acids on the CDR3 heads of Nb_{HSA}. Pearson correlation coefficients and the statistical values are shown. (J) The correlation plot of ELISA affinities and the number of positively charged residues on the CDR2s of Nb_{GST}. (K) Sequence logo of two representative convolutional CDR3 filters (Filter 14 for high-affinity Nb_{HSA}; filter 3 for low-affinity Nb_{HSA}) learned by a deep learning model. The sequence of the top panel of FIG. 5K is SEQ ID NO: 2661 (YXXXXXX, residue 2 can be Y, L, D, R, or I; residue 3 can be K or G; residue 4 can be R, Y, T, or D; residue 5 can be P, D, or R, residue 6 can be E, Y, V, P, W or D; residue 7 can be G, W, D, or P). The sequence of the bottom panel of FIG. 5K is SEQ ID NO: 2662 (YXXXLXX, residue 2 can be D, P, K, or A; residue 3 can be F, P, D, or A; residue 4 can be H, T, or G, residue 6 can be G, N; residue 7 can be R, P, D, or Y.

FIG. 6(A-H): The outstanding versatility of Nbs for antigen binding. (A) The electrostatic potential surface and the dominant E2 epitope of PDZ domain (PDB: 2JIK; E1: 7-8, 35-36, 43, 99-100, and E2: 25-26, 45-46, 48, 78-79, 82-83, 85-86). (B) A docking model by a long CDR3 (in deep salmon) of a high-affinity Nb_{PDZ}P10. (C) Comparison between a crystal structure of PDZ-peptide ligand complex (PDB:1EB9) and a docking model of PDZ-Nb complex. The conserved ligand binding sites are shown in cyan. Side chains of both CDR3 and the peptide ligand are shown. (D) A heatmap showing the ELISA affinities of 11 different Nbs for binding to wild type or a mutant (R46E: K48D) PDZ. * indicates a decrease of 10-100,000 fold ELISA affinity. (E) Plot comparisons of both the CDR3 lengths (upper panel) and pIs (lower panel) of different Nbs (high-affinity Nb_{HSA}, Nb_{GST}, Nb_{PDZ} and Nbs from the sequence database). The data was smoothed with a gaussian function. (F) Comparisons of pI and hydropathy among different Nbs. (G) Pie charts of the top 6 most abundant amino acids on the Nb CDR3 heads. (H) A schematic model for antigen binding by Nbs.

FIG. 7(A-F). Analysis of NGS Nb databases and representative false positive CDR3 peptide identifications. (A) The normalized variability of Nb sequences. Approximately 0.5 million unique Nb sequences were aligned based on IMGT numbering scheme to generate the plot. Amino acids were grouped based on their properties (i.e., positive, negative, polar, and nonpolar) and were color-coded. (B) The mass distribution of ˜1.5 million peptide identifications of human proteins from PeptideAtlas. (C) In silico digestion of Nb NGS database by different proteases (AspN, GluC, LysC, Trypsin and Chymotrypsin) and plot of peptide masses. (D) The overlaps between the target Nb sequence database of the immunized Llama and a decoy database from another native Llama. ˜0.5 million sequences were included in each database. (E) A representative low quality/false positive MS/MS spectrum (HCD) of a tryptic CDR3 peptide. (F) That of a chymotryptic CDR3 peptide. Few high-resolution fragment ions were matched in the spectra. The sequences in FIG. 7E are NTVYLQMNSLKPE (SEQ ID NO: 2658) and DTSIYYCAATPVFQSMSTMATESVYDYWGQGTQVTVSSEPK (SEQ ID NO: 2659). The sequence in FIG. 7F is CAAGSGVGLY (SEQ ID NO: 2660).

FIG. 8(A-J). The informatics pipeline of “Augur Llama” for Nb proteomics and validation of Nb binders. (A) Schematics of the informatic pipeline. Three modules including 1) peptide identifications, 2) Nb peptide and protein quality control, and 3) quantification and classifications were presented. Nb proteomics data is first searched against the search engine. The initial identifications that pass the search engine can be automatically annotated, and evaluated based on different quality filters at peptide and protein levels. High-quality fingerprint peptides that pass the quality filters can be quantified and clustered. (B) Illustrations of the Nb CDR3 spectrum and coverage quality filters. (C) Illustrations of peptide classification method. (D) Phylogenetic tree and Web logo analyses of 230 unique CDR3s of the identified Nb_{PDZ}. (E) Schematic of PCR amplifications of HcAb variable domain (VHH) from B lymphocytes of the camelid. (F) DNA gel electrophoresis of the VHH PCR amplicons from the cDNA libraries prepared from the immunized bone marrow/blood. (G) SDS-PAGE analysis of fractionated Nb_{GST} based on different fractionation protocols. (H) SDS-PAGE analysis of Nb_{PDZ}. Maltose-binding protein (MBP) tag was fused to PDZ domain and the fusion protein was used as affinity handle for isolation. MBP was used as a negative control for quantification. (I) Unique Nb identifications for different antigens. (J) Comparison of antigen-specific Nbs identified by either chymotrypsin or trypsin-based method. Y axis stands for the % of the positive hits that were randomly selected for verifications.

FIG. 9(A-D). Proteomic quantifications, biochemical verifications and affinity measurements of Nb_{GST}. (A) Proteomic quantifications and heatmap analysis of Nb_{GST} based on different fractionation methods. (B) Pearson correlations of LC retention times of different fractionated Nb peptide samples. (C) Representative GST beads-binding assay. GST coupled resin was used to specifically isolate recombinant Nb from the E. coli lysis. Red arrows indicate enriched Nbs. Inactivated resin was used for negative control. (D) SPR kinetic measurements of 10 representative Nb_{GST}.

FIG. 10(A-B). Characterizations of High-quality HSA and PDZ Nbs. (A) SPR kinetic measurements of representative high-affinity Nb_{HSA}. (B) Beads-binding assays of selected high-quality Nb_{PDZ}. Recombinant MBP fusion PDZ was used as an affinity handle for isolation of Nbs from E. coli lysates. MBP coupled resin was used for negative control. I: E. coli lysate input, B: beads control, P: affinity pullout by PDZ.

FIG. 11(A-G). Hybrid structural analysis of GST-Nb complexes. (A) Heatmap analysis of structural docking of 64,670 GST-Nb complexes showing three converged epitopes (E1: 75-88, 143-148; E2: 33-43, 107-127; E3: 158-200, 213-220). (B) Cartoon representations of the three dominant GST epitopes. GST dimers were presented in gray. E1, E2 and E3 were in pale yellow, orange, and deep teal respectively. (C) Surface representations showing colocalizations of electrostatic surfaces with three major epitopes. (D) GST epitopes and their abundances (%) based on converged cross-link models were shown with different colors.

FIG. 12(A-H). The analysis of the CDR sequences of different Nbs and the sequence conservation of camelid and human albumin. (A-B) Comparison of the abundance of amino acids on the CDR3 heads between high-affinity and low-affinity Nbs. (C-F) Comparison of CDR1 and CDR2 for different Nbs. (G) Comparison of the relative position of tyrosine (Y), glycine(G) and serine(S) on the CDR3 heads of GST Nbs. (H) Sequence alignment of human serum albumin and llama serum albumin. Conserved amino acids were highlighted.

FIG. 13(A-F). Comparison among different antigen epitopes. (A) Comparison of the geometries of a major epitope of three different antigens (i.e., E2 for PDZ, E3 for GST dimer and E3 for HSA). Different epitopes were color coded on the antigen structures. (B) The surface electrostatic potentials and the E1 epitope of the PDZ domain. (C) A plot of the solvent accessible areas of different epitopes. The y axis stands for the areas of different epitopes in square angstrom. (D) Net formal charges of the epitopes. (E) Relative abundance of different amino acids on the CDR3 heads. DB: NGS Nb sequence database. (F) Comparison of the pI of CDR1 and CDR2 among different antigen-specific Nbs.

FIG. 14 depicts an example of a computing system that executes methods and procedures described in certain embodiments of the present disclosure.

FIG. 15(A-B) shows the results of amino acid sequence filters that are derived from the deep learning approach. The sequence filters can be used to accurately separate high-affinity from low-affinity binding HSA Nbs. The sequence of FIG. 15A is SEQ ID NO: 2663 (LXYRXXX, residue 2 can be N, Y, V, or G; residue 5 can be L or W; residue 6 can be E, G, N, T, or S; residue 7 can be D or E). The sequence of FIG. 15B is SEQ ID NO: 2664 (XXXXXXX, residue 1 can be C, F, Q, S, H, K, L, Y, or R; residue 2 can be G, P, A, or N; residue 3 can be E, S, G, T, P, V, Y, H, or A; residue 4 can be C, A, S, P, or D; residue 5 can be I, W, V, T, or A; residue 6 can be M, Q, or H; residue 7 can be K, Y, Q, V, or W).

FIG. 16(A-C) shows the results of amino acid sequence filters that are derived from the deep learning approach. The sequence filters can be used to accurately separate high-affinity from low-affinity binding HSA Nbs. The sequence of FIG. 16A is SEQ ID NO: 2665 (TXXXLXX; residue 2 can be D, P, K, or A; residue 3 can be F, P, L, D, or A; residue 4 can be H, T, or G; residue 6 can be G, E, N, or R; residue 7 can be R, P, G, D, or Y). The sequence of FIG. 16B is SEQ ID NO: 2666 (XXRXXXX; residue 1 can be E, G, W, D, or I; residue 2 can be N, G, or C; residue 4 can be A, H, or D; residue 5 can be E, R, Y, A, or T; residue 6 can be G, A, or P; residue 7 can be L, S, or Y). The sequence of FIG. 16C is SEQ ID NO: 2667 (XXGAQXW; residue 1 can be R or A; residue 2 can be K or L; residue 6 can be L, G, Y, or W).

### DETAILED DESCRIPTION

Here reported is an integrative proteomic platform for in-depth discovery, classification, and high-throughput structural characterization of antigen-engaged Nb repertoires. The sensitivity and robustness of the technologies were validated using antigens spanning three orders of magnitude in immune response including a small, weakly immunogenic antigen derived from mitochondrial membrane. Tens of thousands of highly diverse, specific Nb families were confidently identified and quantified according to their physicochemical properties; a significant fraction had sub-nM affinity. Using high-throughput structural modeling, structural proteomics, and deep learning, the structural landscapes of >100,000 antigen-Nb complexes were systematically surveyed to significantly advance the understanding of immunogenicity and Nb affinity maturation. The study has revealed a surprising efficiency, specificity, diversity, and versatility of the mammalian humoral immune system.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “antibody” and “antibodies” are used herein in a broad sense and include polyclonal antibodies, monoclonal antibodies, and bi-specific antibodies. In addition to intact immunoglobulin molecules, also included in the term “antibodies” are fragments or polymers of those immunoglobulin molecules, and human or humanized versions of immunoglobulin molecules or fragments thereof. Antibodies are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

As used herein, the terms “antigen” or “immunogen” are used interchangeably to refer to a substance, typically a protein, a nucleic acid, a polysaccharide, a toxin, or a lipid, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The terms “antigenic determinant” and “epitope” may also be used interchangeably herein, referring to the location on the antigen or target recognized by the antigen-binding molecule (such as the nanobodies of the invention). Epitopes can be formed both from contiguous amino acids (a “linear epitope”) or noncontiguous amino acids juxtaposed by tertiary folding of a protein. The latter epitope, one created by at least some noncontiguous amino acids, is described herein as a “conformational epitope.” An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “antigen binding site”, “binding site” and “binding domain” refer to the specific elements, parts or amino acid residues of a polypeptide, such as a nanobody, that bind the antigenic determinant or epitope.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “cDNA library” refers herein to a combination of different cDNA fragments, which constitute some portion of the transcriptome of a given organism.

The terms “CDR” and “complementarity determining region” are used interchangeably and refer to a part of the variable chain of an antibody that participates in binding to an antigen. Accordingly, a CDR is a part of, or is, an “antigen binding site.” In some embodiments, the nanobody comprises three CDR that collectively form an antigen binding site.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

“Effective amount” encompasses, without limitation, an amount that can ameliorate, reverse, mitigate, prevent, or diagnose a symptom or sign of a medical condition or disorder (e.g., cancer). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant nanobody” refers to an amount of a recombinant nanobody sufficient to prevent, treat, or mitigate a cancer.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to HSA and/or ameliorating cancer.

The term “fragmentation coverage percentage” refers to a percentage obtained using the following formula:

f(x,Enzyme) is the function to calculate fragmentation coverage (%) of peptides digested by Enzyme

x is the length of CDR3 that the peptide mapped

f(x,chymotrypsin)=0.0023x²−0.0497x+0.7723,x[5,30]

f(x,trypsin)=0.00006x²−0.00444x+0.9194,x[5,30].

In some embodiments, a minimum calculated fragmentation coverage percentage is required. In other or further aspects, the required minimum calculated fragmentation coverage percentage is about 30. In some aspects, the required minimum calculated fragmentation coverage percentage is about 50 when trypsin is the enzyme and about 40 when chymotrypsin is the enzyme.

As used herein, a “functional selection step” is a method by which nanobodies are divided into different fractions or groups based upon a functional characteristic. In some embodiments, the functional characteristic is nanobody or CD3, CD2, or CD1 region antigen affinity. In other embodiments, the functional characteristic is nanobody thermostability. In other embodiments, the functional characteristic is nanobody intracellular penetration. Accordingly, the present invention includes a method of identifying a group of complementarity determining region (CDR)3, 2 or 1 region nanobody amino acid sequences (CDR3, CDR2 or CDR1 sequences) wherein a reduced number of the CDR3, CDR2 or CDR1 sequences are false positives as compared to a control, the method comprising: obtaining a blood sample from a camelid immunized with the antigen; using the blood sample to obtain a nanobody cDNA library; identifying the sequence of each cDNA in the library; isolating nanobodies from the same or a second blood sample from the camelid immunized with the antigen; performing a functional selection step; digesting the nanobodies with trypsin or chymotrypsin to create a group of digestion products; performing a mass spectrometry analysis of the digestion products to obtain mass spectrometry data; selecting sequences identified in step c. that correlate with the mass spectrometry data; identifying sequences of CDR3, CDR2 or CDR1 regions in the sequences from step g.; and excluding from the CDR3, CDR2 or CDR1 region sequences from step h. those sequences having less than a calculated fragmentation coverage percentage; wherein the non-excluded sequences comprise a group having the reduced number of false positive CDR3, CDR2 or CDR1 sequences. It should be understood that the method steps following the functional selection step can be performed separately on each different fraction or group created by the functional selection.

The “half-life” of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of a nanobody, amino acid sequence, compound or polypeptide of the invention can be determined in any manner known, such as by pharmacokinetic analysis. these, for example, Kenneth, A et al., Chemical Stability of Pharmaceuticals: A Handbook for Pharmacists; Peters et al., Pharmacokinete analysis: A Practical Approach (1996); “Pharmacokinetics”, M Gibaldi & D Perron, published by Marcel Dekker, 2nd Rev. edition (1982).

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.). In some embodiments, percent identity is determined along the entire length of the compared sequences.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to isolation.

The term “mass spectrometry” refers to a measurement of the mass-to-charge ratio (m/z) of one or more molecules present in a sample. “Mass spectrometry data” refers to mass, charge, mass-to-charge ratio, molecular weight and/or amino acid identity or sequence of the one or more molecules present in a sample. In some embodiments, the mass spectrometry data is the amino acid sequence of a molecule present in the sample. Sequences, including cDNA sequences, that “correlate” with mass spectrometry data have an expected same or highly similar amino acid sequence determined in the mass spectrometry step of the method. In some embodiments, a sequence correlates with mass spectrometry data when there is about 80%, about 85%, about 90%, about 91%, about 92%, about 93%, about 94%, about 95%, about 96%, about 97%, about 98%, or about 99% similarity or identity. In some embodiments, a sequence correlates with mass spectrometry data when there is about 90-100% similarity or identity.

As used herein, the terms “nanobody”, “V_{H}H”, “V_{H}H antibody fragment” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety. As used herein, “single domain antibody” refers to a nanobody and an Fc domain.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

As used herein, “operatively linked” refers to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via a “linker” that comprises one or more intervening amino acids.

The term “reduced”, “reduce”, “reduction”, or “decrease” as used herein generally means a decrease by a statistically significant amount. However, for avoidance of doubt, “reduced” means a decrease by at least 5% as compared to a reference level, for example a decrease by at least about 10%, or at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein. The terms “peptide,” “protein,” and “polypeptide” are used interchangeably herein.

“Recombinant” used in reference to a polypeptide refers herein to a combination of two or more polypeptides, which combination is not naturally occurring.

The term “specificity” refers to the number of different types of antigens or antigenic determinants to which a particular antigen-binding molecule (such as the nanobody of the invention) can bind. A nanobody with low specificity binds to multiple different epitopes (or polypeptide regions) via a single antigen binding site or binding domain, whereas a nanobody with high specificity binds to one or a few epitopes (or polypeptide regions) via a single antigen binding site or binding domain. In some embodiments, the few epitopes (or polypeptide regions) are similar or highly similar, such as, for example, cross-species epitopes. As used herein, the term “specifically binds,” as used herein with respect to a nanobody refers to the nanobody's preferential binding to an epitope (or polypeptide region) as compared with other epitopes (or polypeptide regions). Specific binding can depend upon binding affinity and the stringency of the conditions under which the binding is conducted. In one example, a nanobody specifically binds an epitope when there is high affinity binding under stringent conditions. In some embodiments, the HSA binding polypeptide or nanobody described herein specifically binds to human serum albumin.

It should be understood that the specificity of an antigen-binding molecule (e.g., the HSA binding polypeptides, the nanoantibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (KA), which is 1/K_{D}). Methods for determining affinity are well known to those of ordinary skill in the art. Avidity is the measure of the strength of binding between an antigen-binding molecule (such as the HSA binding polypeptides and the nanobodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the HSA binding polypeptides and the nanobodies of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e., with an association constant (KA) of 10⁵ to 10¹² liter/moles or more, and preferably 10⁷ to 10¹² liter/moles or more and more preferably 10⁸ to 10¹² liter/moles). In some embodiments, the Ka (on rate, IMs) is about 10⁵, 10⁶, 10⁷, 10⁸, 10⁹, 10¹⁰, or 10¹¹. In some embodiments, the Ka is about 10⁷. In some embodiments, the Kd (off rate, s) is about 10⁻⁵, 10⁻⁶, 10⁻⁷, 10⁻⁸, 10⁻⁹, 10⁻¹⁰, or 10⁻¹¹. In some embodiments, the K_{D} is about 10⁻⁷. In some embodiments, the antigen-binding protein disclosed herein binds to its antigen with a K_{D} of less than about 10⁻⁹ moles/liter. Any K_{D} value greater than 10 μM is generally considered to indicate non-specific binding. The dissociation constant may be the actual or apparent dissociation constant, as will be clear to the person of ordinary skill in the art.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

### Compositions and Methods

In some aspects, disclosed herein is a method of identifying a group of complementarity determining region (CDR)3, 2 or 1 region nanobody amino acid sequences (CDR3, CDR2 or CDR1 sequences) wherein a reduced number of the CDR3, CDR2 or CDR1 sequences are false positives as compared to a control. The term “false positive” herein refers to a result that indicates something is present when it is not. Herein the phrase “sequences are false positive” refers to the CDR3, CDR2 and/or CDR1 sequences that do not specifically bind to the tested antigens, or to the CDR3, CDR2 and/or CDR1 sequences contained within a nanobody, which nanobody cannot specifically bind to the tested antigens. It should be understood that the number or amount of false positive CDR3, CDR2 and/or CDR1 sequences can be reduced using the methods disclosed herein with a fragmentation filter set at about at least 30% (for example, at least about 30%, 35%, 40%, 45%, 50%, 55%, 60%, 65%, 70%, 75%, 80%, 85%, 90%, 95%, or 99%) for trypsin-treated samples and/or about at least 30% (for examples, at least about 30%, 35%, 40%, 45%, 50%, 55%, 60%, 65%, 70%, 75%, 80%, 85%, 90%, 95%, or 99%) for chymotrypsin-treated samples. In some examples, the false positive CDR3, CDR2 and/or CDR1 sequences can be mostly removed using the methods disclosed herein with a fragmentation filter set at about 50% for trypsin-treated samples and/or about 40% for chymotrypsin-treated samples.

Accordingly, the disclosed method of identifying CDR3, CDR2 and/or CDR1 sequences can reduce the number of the CDR3, CDR2 and/or CDR1 sequences that are false positives as compared to a control. The reduction can be, for example, at least about a 2-fold, at least about a 3-fold, at least about a 4-fold, at least about a 5-fold, at least about a 10-fold, at least about a 20-fold, at least about a 50-fold, or at least about a 100-fold compared to the number of false positive CDR3, CDR2 and/or CDR1 sequences that are identified without using the method described herein.

In some embodiments, the method comprises:
a. obtaining a blood sample from a camelid immunized with an antigen;
b. using the blood sample to obtain a nanobody cDNA library;
c. identifying the sequence of each cDNA in the cDNA library;
d. isolating nanobodies from the same or a second blood sample from the camelid immunized with the antigen;
e. digesting the nanobodies with trypsin or chymotrypsin to create a group of digestion products;
f. performing a mass spectrometry analysis of the digestion products to obtain mass spectrometry data;
g. selecting sequences identified in step c. that correlate with the mass spectrometry data;
h. identifying sequences of CDR3, CDR2 and/or CDR1 regions in the sequences from step g.; and
i. selecting from the CDR3, CDR2 and/or CDR1 region sequences of step h. those sequences having equal to or more than a required fragmentation coverage percentage; wherein the selected sequences comprise a group having the reduced number of false positive CDR3, CDR2 and/or CDR1 sequences.

In some embodiments, the method comprises:
a. obtaining a blood sample from a camelid immunized with an antigen;
b. using the blood sample to obtain a nanobody cDNA library;
c. identifying the sequence of each cDNA in the library;
d. isolating nanobodies from the same or a second blood sample from the camelid immunized with the antigen;
e. digesting the nanobodies with trypsin or chymotrypsin to create a group of digestion products;
f. performing a mass spectrometry analysis of the digestion products to obtain mass spectrometry data;
g. selecting sequences identified in step c. that correlate with the mass spectrometry data;
h. identifying sequences of CDR3, CDR2 and/or CDR1 regions in the sequences from step g.; and
i. selecting from the CDR3, CDR2 and/or CDR1 region sequences of step h. those sequences having equal to or more than a required fragmentation coverage percentage; wherein the fragmentation coverage percentage is determined by a formula f(x,chymotrypsin)=0.0023x2−0.0497x+0.7723,x[5,30] when chymotrypsin is used in step e. or a formula f(x,trypsin)=0.00006x2−0.00444x+0.9194, x[5,30] when trypsin is used in step e., and wherein x is the length of the CDR3, CDR2 and/or CDR1 region sequence; and
j. wherein the selected sequences of step i. comprise a group having the reduced number of false positive CDR3, CDR2 and/or CDR1 sequences.

In some aspects, the selected CDR3, CDR2 and/or CDR1 region sequences in step i. have a minimum required fragmentation coverage percentage of about 30. In some aspects, the selected CDR3, CDR2 and/or CDR1 region sequences in step i. have a minimum required fragmentation coverage percentage of about 50 and trypsin is used in step e. In some embodiments, the selected CDR3, CDR2 and/or CDR1 region sequences in step i. have a minimum required fragmentation coverage percentage about 40 and chymotrypsin is used in step e.

It should be understood that the nanobody cDNA library in step b. is obtained from a biological sample (e.g., a blood sample or bone marrow) of the immunized subject. In some embodiments, the cDNA library is obtained from the B cells. A cDNA (cloned cDNA or complementary DNA) library is a combination of cDNAs that are produced from mRNAs in a biological sample (e.g., a blood sample or bone marrow sample) using reverse transcription technology. The method of producing cDNA library is well-known in the art. Accordingly, in some embodiments, step b. further comprises a step of isolating mRNAs from a biological sample (e.g., a blood sample or a bone marrow sample) and/or a step of reverse transcribing the isolated mRNA to cDNAs.

The produced cDNAs are then sequenced as described in step c. In some embodiments, step c. further comprises a step of amplifying camelid IgG heavy chain cDNA sequences from the variable domain to the CH2 domain using specific primers (e.g., SEQ ID NO: 2646 and SEQ ID NO: 2647), a step of separating the V_{H}H genes that lack CH1 domain from conventional IgG (having CH1 domain) using DNA gel electrophoresis, a step of re-amplifying from framework 1 to framework 4 using a 2nd-Forward primer (e.g., SEQ ID NO: 2648) and a 2nd-Reverse primer (e.g., SEQ ID NO: 2649), a step of purifying the amplicon of this second PCR (e.g., using a PCR clean up kit or isolation kit), a step of another PCR with primers to add adapter for sequencing analysis (e.g., using forward primer SEQ ID NO: 2650 and reverse primer SEQ ID NO: 2651) for sequencing analysis (e.g., MiSeq sequencing analysis). The methods for sequencing analysis can be, for example, single molecule real time (SMRT) sequencing, nanopore DNA sequencing, massively parallel signature sequencing (MPSS), polony sequencing, 454 pyrosequencing, Illumina (Solexa) sequencing, combinatorial probe anchor synthesis (cPAS), SOLiD sequencing, or MiSeq sequencing.

Step d. above can be performed concurrently, prior, or following steps a, b, and/or c. In some examples, step d. further comprises obtaining plasma from the blood sample and isolating nanobodies using one or more affinity isolation methods. The affinity isolation methods can be any affinity isolation methods known in the art, including, for example, protein G sepharose affinity chromatography, protein A sepharose affinity chromatography, hydroxylapatite chromatography, gel electrophoresis, or dialysis. Protein G sepharose affinity chromatography and protein A sepharose affinity chromatography are two well-known affinity chromatography methods (Grodzki A. C., Berenstein E. (2010) Antibody Purification: Affinity Chromatography—Protein A and Protein G Sepharose. In: Oliver C., Jamur M. (eds) Immunocytochemical Methods and Protocols. Methods in Molecular Biology (Methods and Protocols), vol 588. Humana Press.) The methods rely on the reversible interaction between a protein and a specific ligand immobilized in a chromatographic matrix. The sample is applied under conditions that favor specific binding to the ligand as the result of electrostatic and hydrophobic interactions, van der Waals' forces, and/or hydrogen bonding. After washing away the unbound material, the bound protein is recovered by changing the buffer conditions to those that favor desorption. Protein A sepharose affinity chromatography and G sepharose affinity chromatography are commonly used in antibody purification due to the high binding affinity and specificity of Protein A or G with the Fc region of the antibody. In some embodiments, the one or more affinity isolation methods of step d. comprise one or more of protein G sepharose affinity chromatography and protein A sepharose affinity chromatography.

In some examples, step d. also further comprises a functional selection step comprising selecting antigen-specific nanobodies using an antigen-specific affinity chromatography and eluting the antigen-specific nanobodies under varying degrees of stringency thereby creating different nanobody fractions, and performing steps e. through i. on each fraction individually and estimating an affinity of each different step i. CDR3, CDR2 and/or CDR1 region sequence for the antigen based on a relative abundance of the CDR3, CDR2 and/or CDR1 region sequence in each of the nanobody fractions, respectively. In some embodiments, the antigen-specific affinity chromatography is a resin conjugated to the antigen. In some embodiments, the antigen-specific affinity chromatography is a resin coupled to maltose binding protein and the antigen.

It should be understood and herein contemplated that the term “degrees of stringency” refers to different concentrations of salt buffer (e.g., from about 0.1M to about 20 M MgCl₂ in neutral pH buffer, preferably from about 1 M to about 10 M MgCl₂ in neutral pH buffer, or preferably from about 1M to about 4.5 M MgCl₂ in neutral pH buffer), alkaline solutions with different pH values (e.g., 1-100 mM NaOH, about pH 11, 12 and 13), acidic solutions with different pH values (e.g., 0.1 M glycine, about pH 3, 2 and 1), or a combination thereof. It should also be understood that the term “different nanobody fractions” or “different biochemistry fractions” refers to different fractions of nanobodies that are eluted from an antigen-coupled solid support (e.g., a resin) under the different degrees of stringency. The nanobodies that are most resistant to high salt, high acidity or high alkalinity conditions have the highest affinity to the antigen.

The term “digestion products” herein, such as in step e., refers to the mixture of peptides following the step of digestion with an enzyme (including, for example, trypsin, chymotrypsin, LysC, GluC, and AspN). In some examples, the nanobodies are digested with trypsin(such as Pierce™ Trypsin Protease, MS Grade, Catalog number: 90057), chymotrypsin (such as Pierce™ Chymotrypsin Protease (TLCK treated), MS Grade, Catalog number: 90056), LysC (or Lys-C protease, such as Pierce™ Lys-C Protease, MS Grade, Catalog number:. 90051), GluC (or Glu-C Protease, such as Pierce™ Glu-C Protease, MS Grade, Catalog number:. 90054), and/or AspN (or Asp-N protease, such as Pierce™ Asp-N Protease, MS Grade, Catalog number: 90053) to create the corresponding digestion products. Trypsin, chymotrypsin, LysC, GluC, and AspN are enzymes that digest proteins. The cleavage rules for digestion of nanobodies by these enzymes are:
Trypsin: C-terminal to K/R, not followed by P
Chymotrypsin: C-terminal to W/F/UY, not followed by P
GluC: C-terminal to D/E, not followed by P
AspN: N-terminal to D
LysC: C-terminal to K

The digestion step can be performed at a temperature from about 2° C. to about 60° C. (e.g., at about 2° C., 4° C., 6° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., or 60° C.) for about 5 min, 10 min, 30 min, 45 min, 1 hour, 2 hours, hours, 4 hours, 6 hours, 8 hours, 10 hours, 12 hours, 14 hours, 16 hours, 18 hours, 20 hours, 22 hours, 24 hour, 36 hours, 48 hours, or 72 hours.

Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | s |
| Threonine | Thr | T |
| Tyrosine | Tyr | Y |
| Tryptophan | Trp | W |
| Valine | Val | V |

Step f. comprises performing a mass spectrometry analysis of the digestion products to obtain mass spectrometry data. The methods of using mass spectrometry for peptide analysis are well-known in the art. In some embodiments, the mass spectrometry analysis herein is performed in combination with gas chromatography (GC-MS), liquid chromatography (LC-MS), capillary electrophoresis (CE-MS), ion mobility spectrometry-mass spectrometry (IMS/MS or IMMS), Matrix Assisted Laser Desorption Ionisation (MALDI-TOF), Surface Enhanced Laser Desorption Ionization (SELDI-TOF), or Tandem MS (MS-MS). This step can identify the sequence of the nanobody, or a portion of a nanobody in the sample, based on mass of the amino acids and sequence homology search in a database of polypeptides translated from the cDNA library of step b. In some examples, mass spectrometry is used to analyze and generate a spectrum of digestion products from each nanobody fraction separately. In some examples, the spectrum of the digestion productions refers to the electron ionization data that are present as intensity versus m/z (mass-to-charge ratio) plot.

It should be understood herein that the nanobody sequence determination is not only based on mass spectrometry. It is determined by matching/correlating the sequences identified by mass spectrometry with the sequences the cDNA library identified by sequencing. The matched sequences are then selected. Accordingly, step g. comprises selecting sequences identified in step c. that correlate with the mass spectrometry data and step h comprises identifying sequences of CDR3 regions in the sequences from step g.

Step i. comprises selecting from the CDR3, CDR2 and/or CDR1 region sequences of step h. those sequences having equal to or more than a required fragmentation coverage percentage. In some embodiments, the fragmentation coverage percentage is equal to or more than about 30% (for example, about 30%, 35%, 40%, 45%, 50%, 55%, 60%, 65%, 70%, 75%, 80%, 85%, 90%, 95%, or 99%) for trypsin-treated samples. In some embodiments, the fragmentation coverage percentage is equal to or more than about 30% (for examples, at least about 30%, 35%, 40%, 45%, 50%, 55%, 60%, 65%, 70%, 75%, 80%, 85%, 90%, 95%, or 99%) for chymotrypsin-treated samples. In some embodiments, the fragmentation coverage percentage is about 50% for trypsin-treated samples and about 40% for chymotrypsin-treated samples.

In some embodiments, the method described herein further comprises creating a nanobody comprising a CDR3, CDR2 and/or CDR1 region having a sequence identified in step i. The nanobody genes are cloned into a vector, which is then transformed into competent cells for nanobody protein expression, extraction and purification.

In some embodiments, the nanobody comprises an amino acid sequence at least 80% (for examples, at least about 80%, 85%, 90%, 95%, 98% or 99%) identical to a sequence selected from the group consisting of SEQ ID NOs: 1-157. In some embodiments, the nanobody has a sequence selected from the group consisting of SEQ ID NOs: 1-157. In some embodiments, the nanobody comprises an amino acid sequence at least 80% (for examples, at least about 80%, 85%, 90%, 95%, 98% or 99%) identical to a sequence selected from the group consisting of SEQ ID NOs: 158-2536. In some embodiments, the nanobody has a sequence selected from the group consisting of SEQ ID NOs: 158-2536. In some embodiments, the nanobody comprises an amino acid sequence at least 80% (for examples, at least about 80%, 85%, 90%, 95%, 98% or 99%) identical to a sequence selected from the group consisting of SEQ ID NOs: 2665-2667. In some embodiments, the nanobody has a sequence selected from the group consisting of SEQ ID NOs: 2665-2667.

Disclosed herein is a PDZ-specific nanobody, wherein the PDZ-specific nanobody comprises an amino acid sequence selected from the group consisting of SEQ ID NOs: 158-2536. Also disclosed herein is a PDZ-specific nanobody, wherein the PDZ-specific nanobody comprises an amino acid sequence selected from the group consisting of SEQ ID NOs: 143-157. As used herein, “PDZ” refers to an 80-100 amino acid domain found in signaling proteins that have also been referred to as DHR (Dlg homologous region) or GLGF (glycine-leucine-glycine-phenylalanine) domains. PDZ domains bind to a short region of the C-terminus of other specific proteins. PDZ domains are conventionally divided into three different classes, categorized by the chemical nature of their ligands. Different ligand classes are distinguished by differences in the penultimate binding residues found at the extreme COOH of target proteins. Type I domains recognize the sequence, X-S/T-X-Φ* (where X=any amino acid, Φ=hydrophobic amino acid, * COOH terminus). Type II domains bind to ligands with the sequence X-Φ-X-Φ*. Type III domains interact with sequences with X-X-C*. Binding specificity within each domain class can be conferred by the variant (X) residues as well as residues outside the canonical binding motif. Moreover, a few PDZ domains do not fall into any of these specific classes. Proteins that contain PDZ domains include, but are not limited to, Erbin, GRIP, Htra1, Htra2, Htra3, PSD-95, SAP97, CARD10, CARD11, CARD14, PTP-BL, and SYNJ2BP. In some embodiments, the PDZ domain is from SYNJ2BP.

Disclosed herein is a GST-specific nanobody, wherein the GST-specific nanobody comprises an amino acid sequence in Table 4. Also disclosed herein is a GST-specific nanobody, wherein the GST-specific nanobody comprises an amino acid sequence selected from the group consisting of SEQ ID NOs: 1-98. “Glutathione S-transferase” or “GST” refers herein to glutathione-S-transferases (GSTs) are a family of Phase II detoxification enzymes that catalyze the conjugation of glutathione (GSH) to a wide variety of endogenous and exogenous electrophilic compounds. In some embodiments, the GST polypeptide is that in the pGEX6p-1 vector.

Disclosed herein is a HSA-specific nanobody, wherein the HSA-specific nanobody comprises an amino acid sequence in Table 5. Also disclosed herein is a HSA-specific nanobody, wherein the HSA-specific nanobody comprises an amino acid sequence selected from the group consisting of SEQ ID NOs: 99-142. “Human serum albumin” or “HSA” refers herein to a polypeptide encoded by the ALB gene. In some embodiments, the HSA polypeptide is that identified in one or more publicly available databases as follows: HGNC: 399, Entrez Gene: 213, Ensembl: ENSG00000163631, OMIM: 103600, UniProtKB: P02768. In some embodiments, the HSA polypeptide comprises the sequence of SEQ ID NO: 2668, or a polypeptide sequence having at or greater than about 80%, about 85%, about 90%, about 95%, or about 98% homology with SEQ ID NO: 2668, or a polypeptide comprising a portion of SEQ ID NO: 2668. The HSA polypeptide of SEQ ID NO: 2668 may represent an immature or pre-processed form of mature HSA, and accordingly, included herein are mature or processed portions of the HSA polypeptide in SEQ ID NO: 2668.

Here a robust proteomic pipeline was developed for large-scale quantitative analysis of antigen-engaged Nb proteomes and epitope mapping based on high-throughput structural characterization of antigen-Nb complexes.

### EXAMPLES

### Example 1. The Superiority of Chymotrypsin for Large-Scale Nb Proteomics Analysis

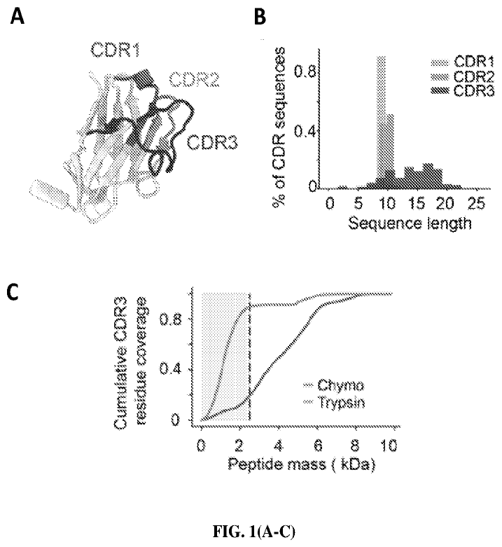

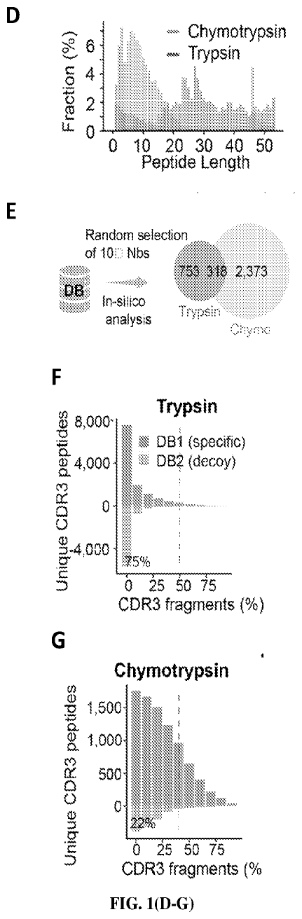

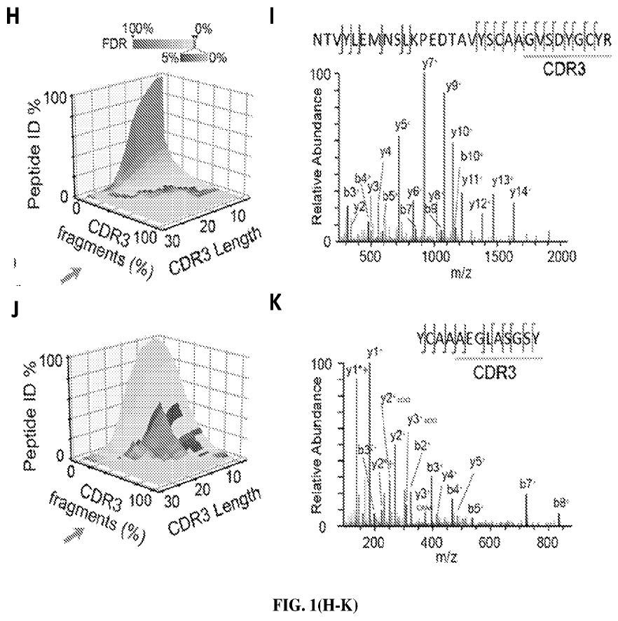

The variable domains of HcAb (VHH/Nb) cDNA libraries were amplified from the B lymphocytes of two lama glamas, recovering 13.6 million unique Nb sequences in the databases by the next-generation genomic sequencing (NGS) (DeKosky, 2013). Approximately half a million Nb sequences were aligned to generate the sequence logo (FIG. 1A,7A). CDR3 loops have both the largest sequence diversity and length variation providing excellent specificity for Nb identifications (FIG. 1B, 1C). In silico analysis of Nb databases revealed that trypsin predominantly produced large CDR3 peptides due to the limited number of trypsin cleavage sites on Nbs (FIG. 1A). As a result, the majority of the CDR3 residues (77%) were covered by large tryptic peptides of more than 2.5 kDa (FIG. 1D, 1E), which are suboptimal for proteomic analysis (FIG. 7B). In comparison, chymotrypsin, which is infrequently used for proteomics cleaving specific aromatic and hydrophobic residues, appears to be more suitable (Methods, FIG. 1A, 7B). 91% of CDR3 sequences can be covered by chymotryptic peptides less than 2.5 kDa (FIG. 1D, 1E). Random selection and simulation confirmed that significantly more CDR3 sequences can be covered by chymotrypsin than trypsin (FIG. 1F). Moreover, there was a small overlap (˜9%) between the two enzymes, indicating their good complementarity for efficient Nb analysis.

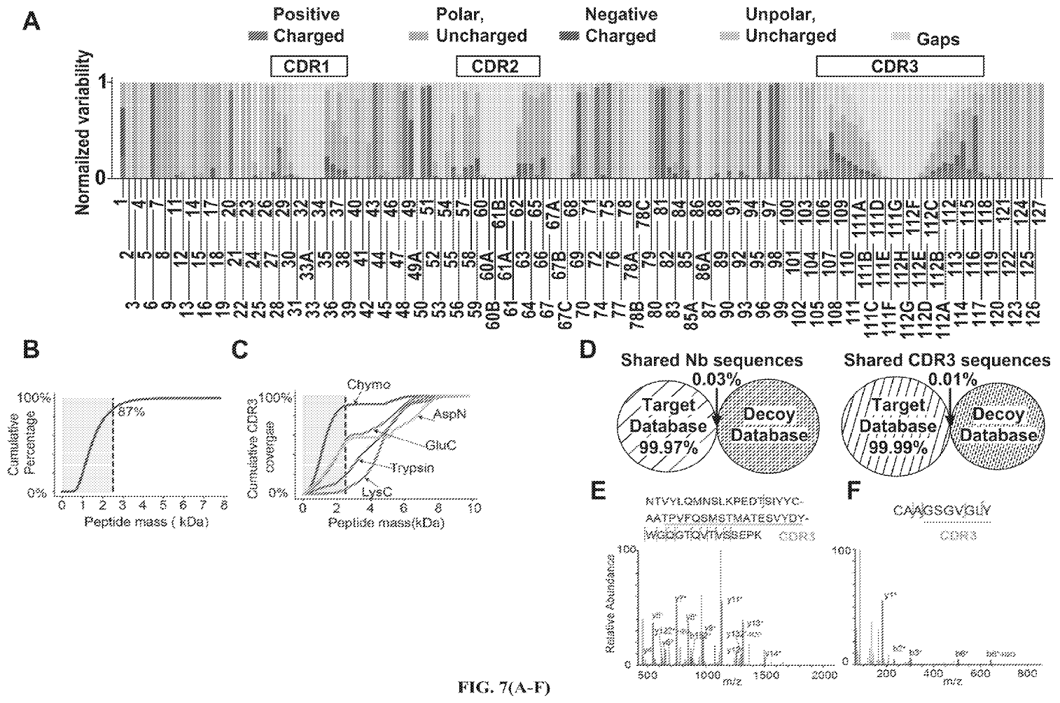

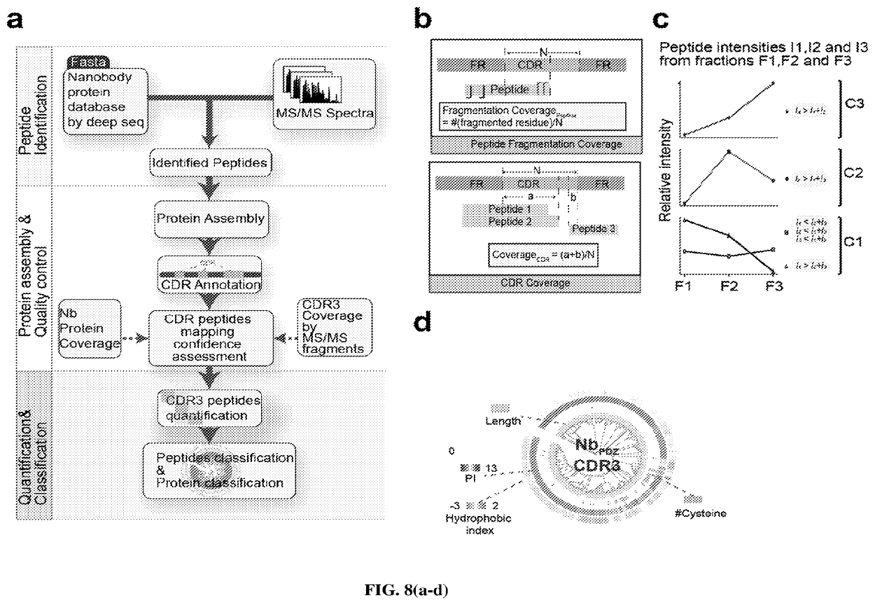

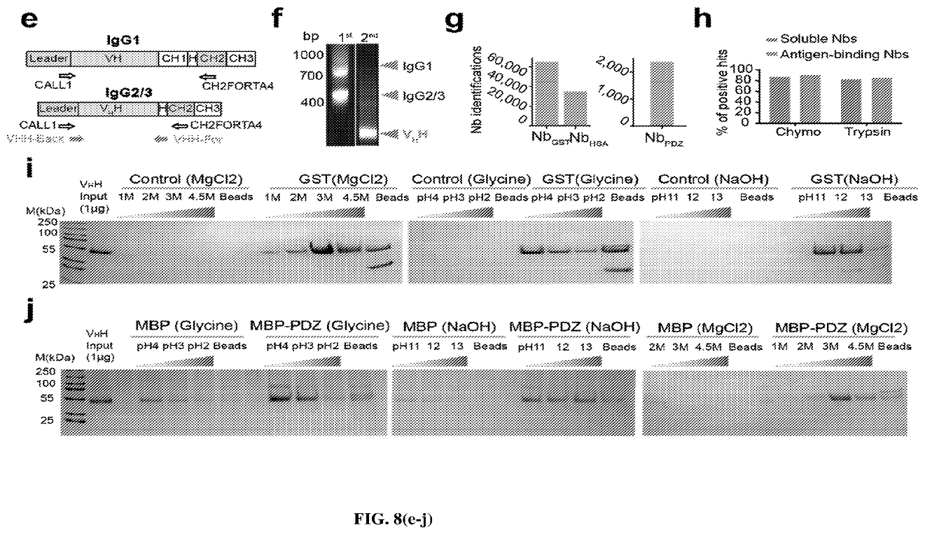

The estimated false discovery rate (FDR) of CDR3 identifications can be inflated due to the large database size and the unusual Nb sequence structure. To test this, antigen-specific HcAbs were proteolyzed with trypsin or chymotrypsin, and a state-of-the-art search engine was employed for identification using two different databases: a specific “target” database derived from the immunized llama, and a “decoy” database of similar size from an irrelevant llama with literally no identical sequences (FIG. 7D). Any CDR3 peptides identified from the decoy database search were thus considered as false positives (Elias, J. E. & Gygi, S. P, 2007). A large number of false positive CDR3 peptides were nonspecifically identified from the decoy database search. It was found that these spurious peptide-spectrum-matches generally contained poor MS/MS fragmentations on the CDR3 fingerprint sequences (FIG. 7E, 7F). The vast majority (95%) of these erroneous matches can be removed by using a simple fragmentation filter that we have implemented, requiring a minimum coverage of 50% (by trypsin, FIG. 1G) and 40% (by chymotrypsin, FIG. 1H) of the CDR3 high-resolution diagnostic ions in the MS2 spectra (FIG. 1K, 1L). The filter was further optimized based on the CDR3 length (FIG. 1I, 1J) before integrating into the new, open-source software “Augur Llama” (FIG. 8A-8C) for reliable Nb proteomic analysis.

### Example 2. Development of an Integrative Proteomics Pipeline for Nb Discovery and Characterization

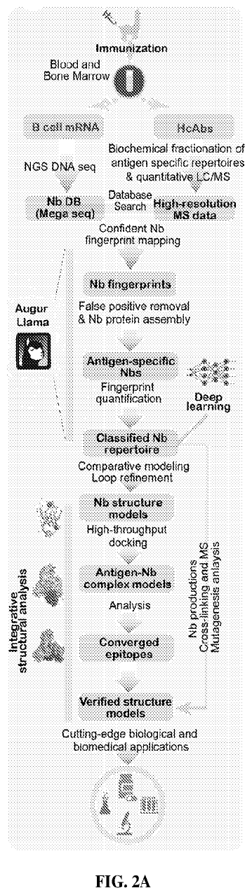

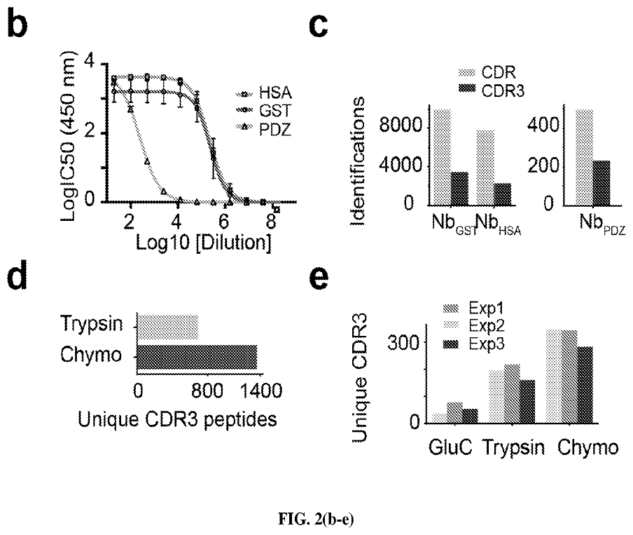

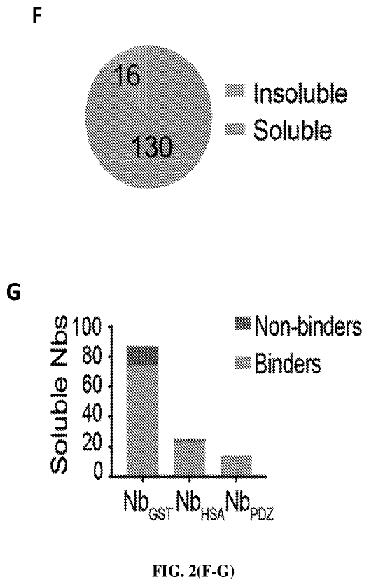

A robust platform is shown herein for comprehensive quantitative Nb proteomics and high-throughput structural characterizations of antigen-Nb complexes (Methods, FIG. 2A). A domestic camelid was immunized with the antigens of interest. The Nb cDNA library was then prepared from the blood and/or bone marrow of the immunized camelid (Fridy, 2014). NGS was performed to create a rich database of >10′ unique Nb protein sequences (FIG. 8E, 8F). Meanwhile, antigen-specific V_{H}Hs were affinity isolated from the sera and eluted using step-wise gradients of salts or pH buffers. Fractionated HcAbs were efficiently digested with trypsin or chymotrypsin to release Nb CDR peptides for identification and quantification by nanoflow liquid chromatography coupled to high-resolution MS. Initial candidates that pass database searches were annotated for CDR identifications. CDR3 fingerprints were filtered to remove false positives, their abundances from different biochemical fractions were quantified to infer the Nb affinities, and assembled into Nb proteins—all of the above steps were automated by Augur Llama. The pipeline enables identification and characterization of an unprecedented scale of diverse, specific, and high-quality Nbs. In parallel, to enable structural analysis of tens of thousands of antigen-Nb interactions, a robust method have been developed to integrate high-throughput computational docking (Schneidman-Duhovny, 2005), cross-linking and mass spectrometry (CXMS) (Chait, 2016; Rout, 2019; Yu, 2018; Leitner, 2016), and mutagenesis. A deep-learning approach was further developed to learn the latent features associated with the Nb repertoires.

### Example. 3. Robust, In-Depth, and High-Quality Identifications of Antigen-Specific Nbs

To validate this pipeline, three benchmark antigens were chosen: glutathione S-transferase (GST), human serum albumin (HSA)-an important drug target (Larsen, 2016), and a small PDZ domain derived from mitochondrial outer membrane protein 25. These antigens span three orders of magnitude of immune responses with PDZ only weakly immunogenic (FIG. 2B) and are ideal to assess the robustness of our technologies.

Here 64,670 unique Nb_{GST} sequences (9,915 unique CDR combinations from 3,453 CDR3 Nb families), 34,972 unique Nb_{HSA} (7,749 unique CDRs from 2,286 unique CDR3 Nb families) and a smaller cohort of 2,379 high-quality Nb_{PD}z sequences (495 unique CDRs from 230 CDR3 families) were identified (Methods, FIG. 2C, 8G). It was confirmed that chymotrypsin provided the most useful fingerprint information for Nb identification from the various proteases tested (FIG. 2D, 2E). The Nb repertoires exhibited exceptional CDR3 diversity (FIG. 8D).

A random set of 146 Nbs was selected from among the three antigen-specific Nb groups and expressed in E. coli. A group of 130 Nbs (89%) exhibited excellent solubility and can be readily purified in large quantities (FIG. 2F). Complementary approaches were taken, including immunoprecipitation, ELISA, and SPR, to evaluate the antigen binding (Methods, FIG. 2G, 9C, 9D, 10, Tables 1-3). Nbs identified by trypsin and chymotrypsin were comparably high-quality (FIG. 8H). 86.2% (CI_{95%}: 6.8%), 90.5% (CI_{95%}: 11.5%), and 100% true Nb binders were confirmed for GST, HSA and PDZ, respectively. These results demonstrate the high sensitivity and specificity of this approach.

### Example 4. Accurate Large-Scale Quantification and Clustering of Nb Proteomes

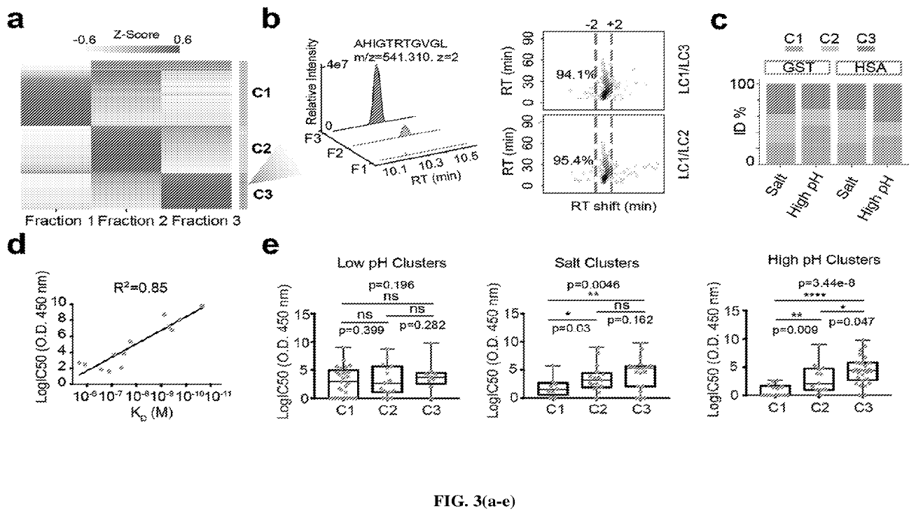

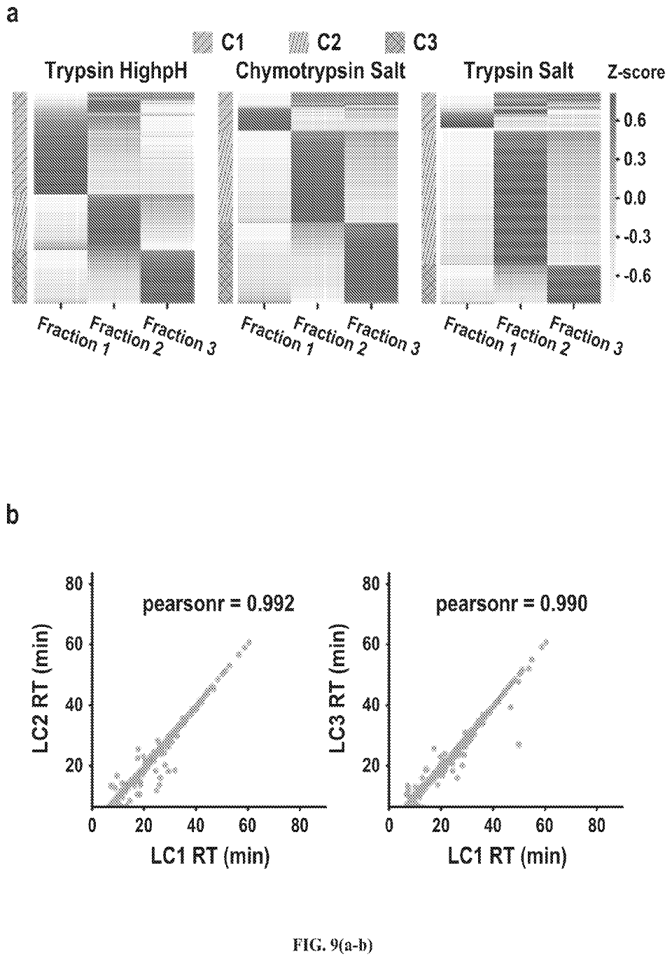

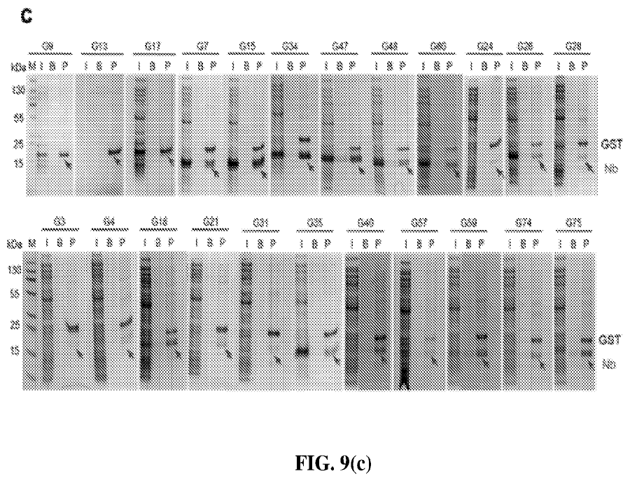

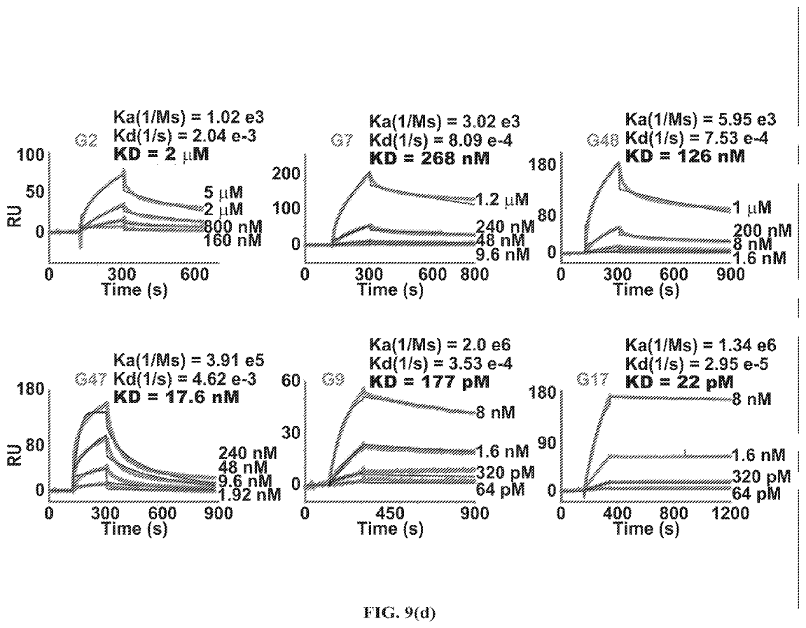

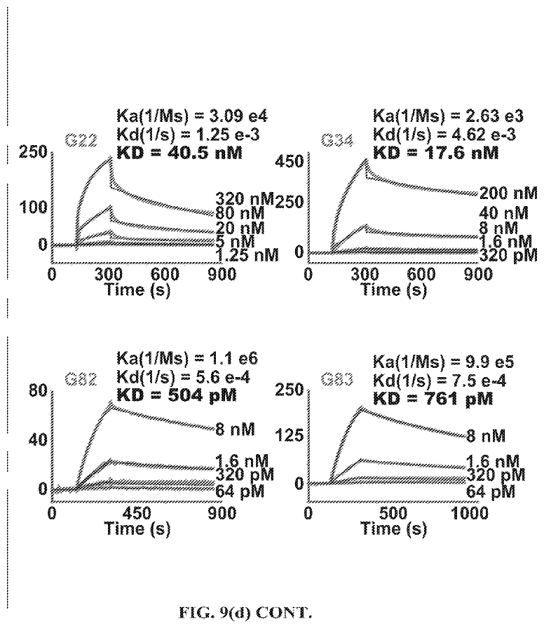

Different strategies were evaluated for accurate classification of Nbs based on affinities. Briefly, antigen-specific HcAbs were affinity isolated from the serum and eluted by the step-wise high-salt gradients, high pH buffers, or low pH buffers (Methods, FIG. 8I, 8J). Different HcAbs fractions were accurately quantified by label-free quantitative proteomics (Zhu, 2010; Cox, J. & Mann, M, 2008). The CDR3 peptides (and the corresponding Nbs) were then clustered into three groups based on their relative ion intensities (FIGS. 3A, 3B, 9A, and 9B). This classification assigns 31% of Nb_{GST} and 47% of Nb_{HSA} into the C3 high affinity group by the high pH method (FIG. 3C). A number of Nb_{GST} with unique CDR3 sequences from each cluster were randomly expressed and their affinities were measured by ELISA and SPR (R²=0.85, FIG. 3D, Table 1) to evaluate different fractionation methods. While the low pH method did not provide sufficient resolution to separate different affinity groups, the salt gradient and particularly the high pH method, enabled significant and reproducible separations of Nbs based on their affinities (FIG. 3E). Nbs from high pH clusters 1 and 2 (C1, C2) generally have low and mediocre affinities, respectively, from μM to dozens of nM, while over 50% of C3 were ultrahigh affinity, sub-nM binders (FIG. 3H, 9D). To further verify this result, a random set of 25 Nb_{HSA} (with divergent CDR3s) were purified from C3, and ranked their ELISA affinities (FIG. 3F, Table 2). The top 14 Nb_{HSA} were selected for SPR measurements, in which 11 have dozens to hundreds of pM affinities with diverse binding kinetics. The remaining 3 Nb_{HSA} demonstrated single-digit nM K_{D}'s. (FIG. 3I, 10A). 13 soluble Nb_{PDZ} were purified and their high affinities were confirmed by ELISA and immunoprecipitation (FIG. 3G, 10B, and Table 3). The K_{D} of a representative, highly soluble Nb_{PDZ} P10 was 4.4 μM (FIG. 3J).

The ultrahigh affinity Nbs for immunoprecipitation (Nb_{GST}) and fluorescence imaging (Nb_{PDZ}) of native mitochondria (FIG. 3K, 3L) were further positively evaluated. The quantitative approach enables large-scale and accurate classification of Nb proteomes based on desirable properties such as affinities.

### Example 5. The Landscapes of Antigen-Engaged Nb Proteomes Revealed by Integrative Structure Determination Methods

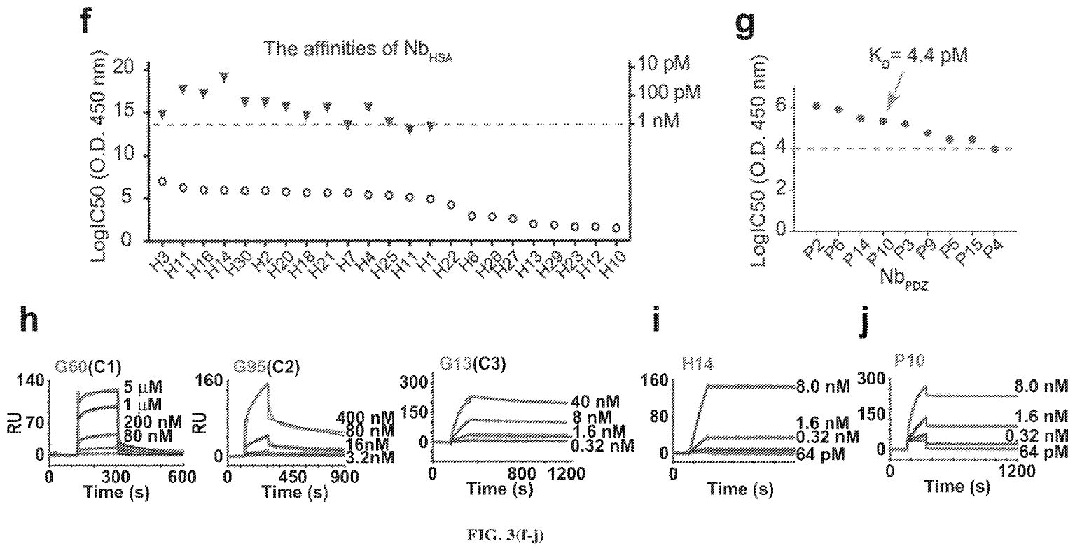

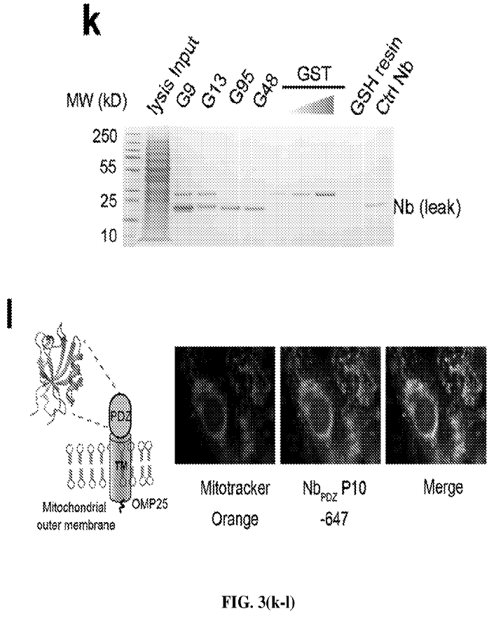

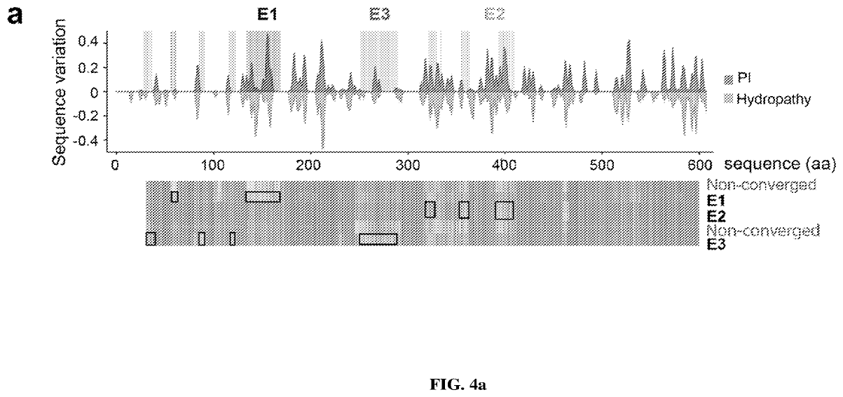

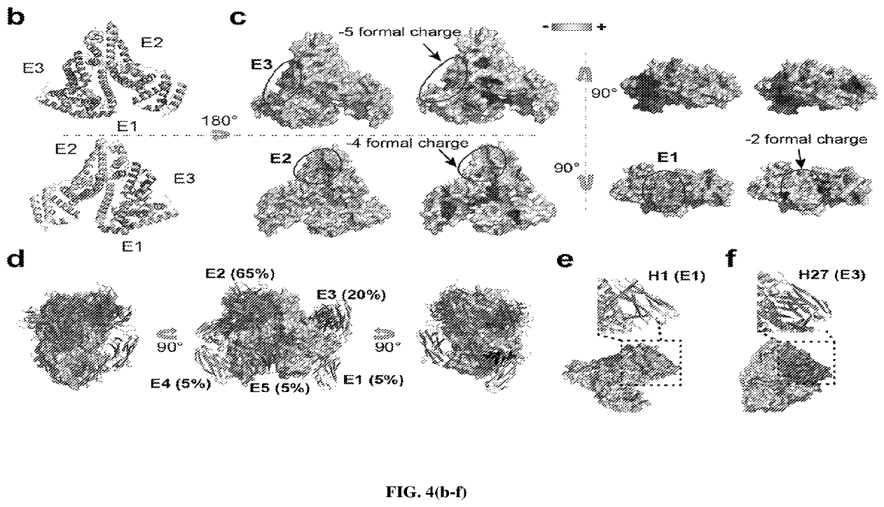

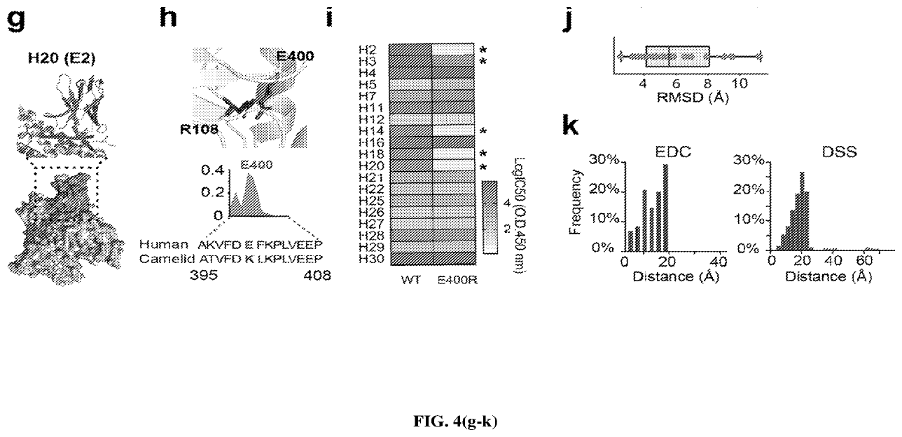

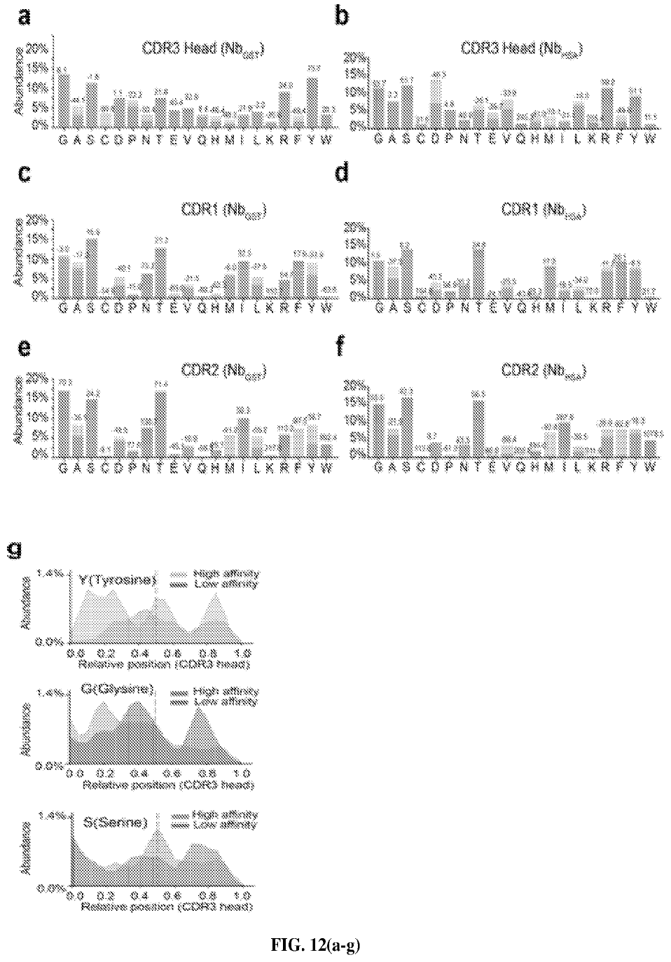

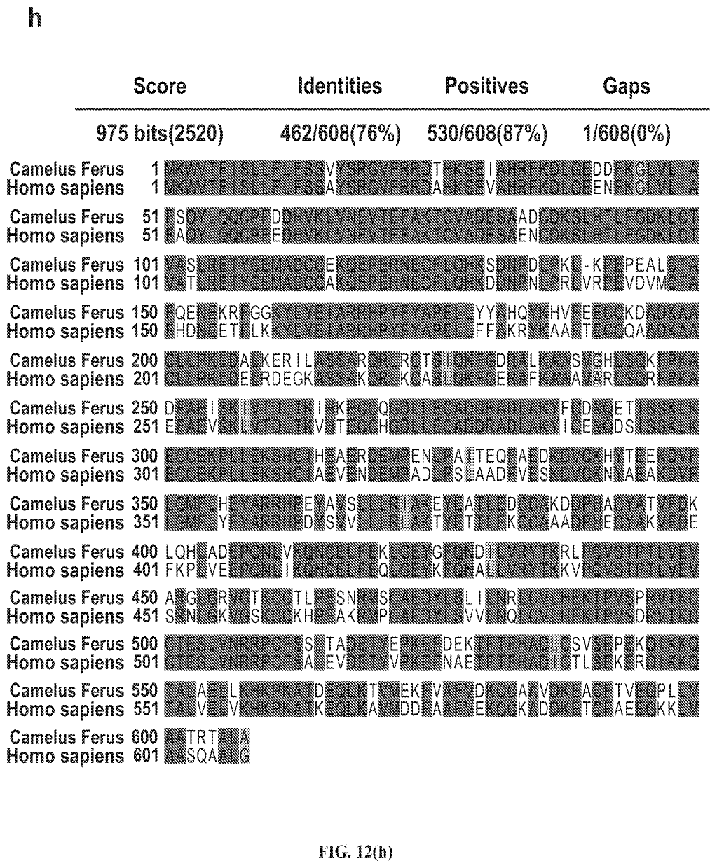

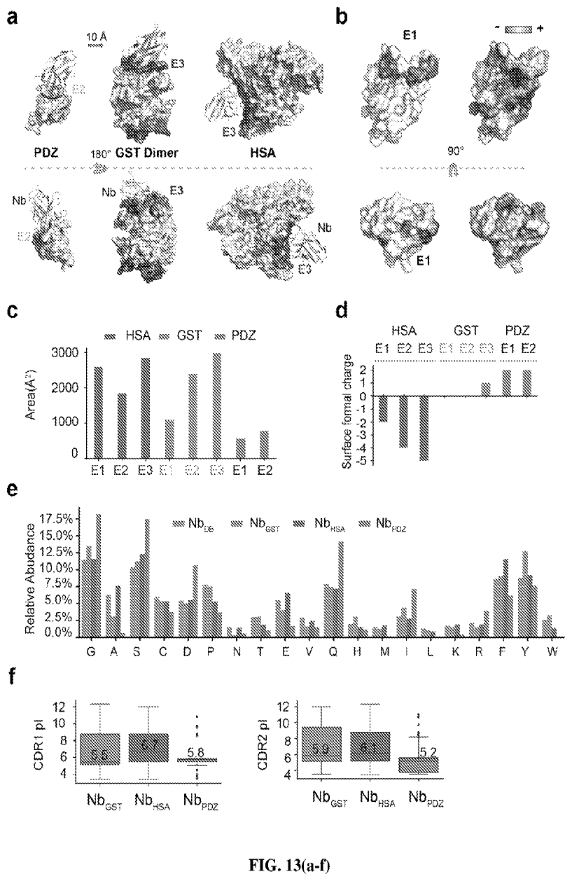

Identification and classification of large repertoires of high-quality Nbs allow to the investigation on the global structure landscapes of antigen-engaged humoral immune response. Structural docking and clustering of 34,972 Nb_{HSA} revealed three dominant HSA epitopes (FIG. 4A). The presence of abundant native serum albumin (76% identical to HSA, FIG. 12H) allowed the investigation on the specificity of the camelid humoral immunity. The two albumin sequences were aligned and their variations were calculated based on pI and hydropathy (Methods, FIG. 4A). All three epitopes are co-localized with the major peaks of pI and hydropathy which correspond to the large sequence differences. This result illustrates the exceptional specificity of antigen recognition by Nbs. It appears that Nbs preferentially bind stable helical secondary structures (FIG. 4B). It was found that the epitopes were highly charged. E2 and E3 were predominantly negative (−4 and −5 net formal charges respectively, FIG. 13D), while E1 was more heterogeneous with mixed charges −2 net formal charges) (FIG. 4C).

19 HSA-Nb complexes (Shi, 2014; Kim, 2018) were cross-linked to verify the epitopes identified by docking. Overall, 92% of cross-links were satisfied by the models, which have a median RMSD of 5.6 Å (FIG. 4J, 4K). Cross-linking confirmed the docking results and identified two epitopes (E2, E3) that were heavily populated (65% and 20%, respectively) (FIG. 4D, Table 2). E1 was identified by cross-links with low abundance (5%). Cross-linking also identified additional two minor epitopes that were not revealed by docking (FIG. 4D). High shape complementarity was observed between HSA and Nbs involving convex Nb paratopes and concave HSA epitopes (FIG. 4E-4G). To further confirm the dominant E2, we introduced a single point mutation on HSA, E400R with minimal impact on the overall structure (Pires, 2016). The resulting mutation reverses the surface charge to mimic the positive charge at the orthologous position in E2 of camelid albumin, potentially disrupting a salt bridge formed between it and an arginine in the Nb CDR3 (FIG. 4H). 19 high-affinity binders were then selected and this point mutation on HSA-Nb interactions was evaluated by ELISA (FIG. 4I, Table 2). E400R almost completely abolished the binding of 5 out of 19 Nbs (26%) that were tested, indicating that E2 is a bonafide major epitope.

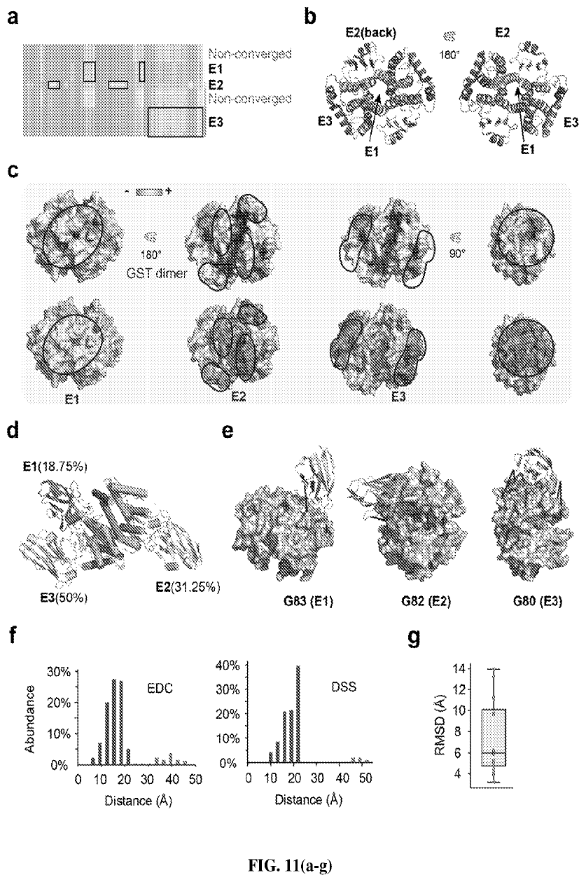

This approach was further employed to map the epitopes of 64,670 GST-Nb complexes. Three major epitopes on GST were accurately identified (FIG. 11A, 11B, 11F, 11G) and were verified by cross-links with relative abundances of 18.75%, 31.25%, and 50% for E1, E2, and E3, respectively (FIGS. 11D, 11E). E1 and E3 contain negatively charged surface patches. E2 overlapped with GST dimerization cavity (FIG. 11C); in the models shown herein E2 Nbs insert their CDR3s into this cavity. Similar to HSA, preference to charged surface residues and high shape complementarity of Nbs were confirmed. Together, these results indicate that Nbs can bind diverse protein surfaces and prefer highly charged cavities on the antigen.

### Example 6. Exploring the Mechanisms of Nb Affinity Maturation

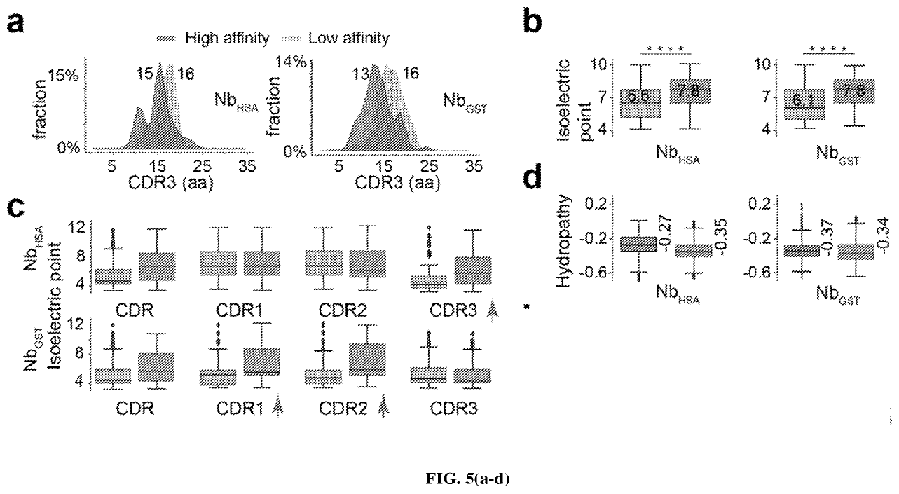

The physicochemical and structural features that distinguish high-affinity (matured) and low-affinity Nbs were investigated, based on the high pH dataset that was most reliably classified. Shorter CDR3s with distinct distributions for high-affinity binders for HSA and GST, respectively (FIG. 5A), lowering the entropy for antigen binding. A significant increase of pI was observed (FIG. 5B), from slightly acidic for low-affinity to relatively basic for high-affinity Nbs.

The contribution of CDRs to pI and hydropathy of the Nbs were compared, and it was determined that CDR3_{HSA} was primarily responsible for polarity shifts in Nb_{HSA} while CDR1_{GST} and CDR2_{GST} were primarily responsible for polarity shifts in Nb_{GST} (FIG. 5C). It was observed that high-affinity Nbs are slightly more hydrophilic (FIG. 5D).

The structure of a CDR3 can be considered as having a “head” region consisting of the highest sequence variability, and a “torso” region of lower specificity (Finn, 2016) (FIG. 5E). Certain residues were enriched on CDR3 heads, including aspartic acid and arginine (forming strong electrostatic interactions) (Tiller, 2017), small and flexible residues of glycine and serine, hydrophobic residues such as alanine and leucine, and aromatic residue of tyrosine (FIG. 5F, and FIG. 12). Nbs of different affinity groups were compared and three major differences were found. First, high-affinity Nbs were more enriched with charged residues (Mitchell, L. S. & Colwell, L J, 2018) (Methods, FIG. 5G). Second, intricate differences were identified for different antigens: high-affinity Nb_{HSA} tend to strengthen the electrostatics by increasing positively charged residues (39%) and decreasing (46%) negatively charged residues on the CDR3 heads. High-affinity Nb_{GST} predominantly altered their charges on other CDRs. Increases of 29.2% and 117.2% of positively charged residues and decreases of 44.2% and 21.5% of negatively charged residues were found on CDR1 and CDR2, respectively. The changes in charge may increase the physicochemical complementarity between the Nb and the epitope. Third, tyrosine (51%), glycine and serine (58%) were more enriched on CDR3 heads for high-affinity Nb_{HSA}. For high-affinity Nb_{GST}, there was an increase in tyrosines (73%) in CDR3 heads but the fractions of glycine and serine were hardly affected.

To further explore the putative roles of these residues for augmenting HSA binding affinity, their location frequency was calculated along the CDR3 heads (FIG. 5H). Tyrosine is more frequently found at the center of CDR3 heads for high-affinity Nb_{HSA} enabling its bulky, aromatic side chain to insert into specific epitope pocket(s) (Desmyter, 1996; Li, 2016). Glycine and serine tend to be placed away from the CDR3 center, providing additional flexibilities and facilitating the orientation of the tyrosine side chain in the antigen pocket. These results were confirmed by the correlation analysis between the number of these residue groups and ELISA affinities of our purified Nbs (FIG. 5I, 5J).

A deep learning model was developed to learn the latent features that enable Nb affinity classification (Methods). The most informative Nb_{HSA} CDR3 filter for high-affinity binder classification revealed a pattern of consecutive lysine and arginine, tyrosines and glycines (FIG. 5K, Table 4). For low-affinity binders, the most informative filter has preference for phenylalanine, histidine, and two consecutive aspartic acids. Moreover, this analysis revealed a tendency for consecutive pairs of negative and positive charges for high- and low-affinity binders, respectively.

### Example 7. The Outstanding Versatility and Resilience of Nbs for Antigen Recognition

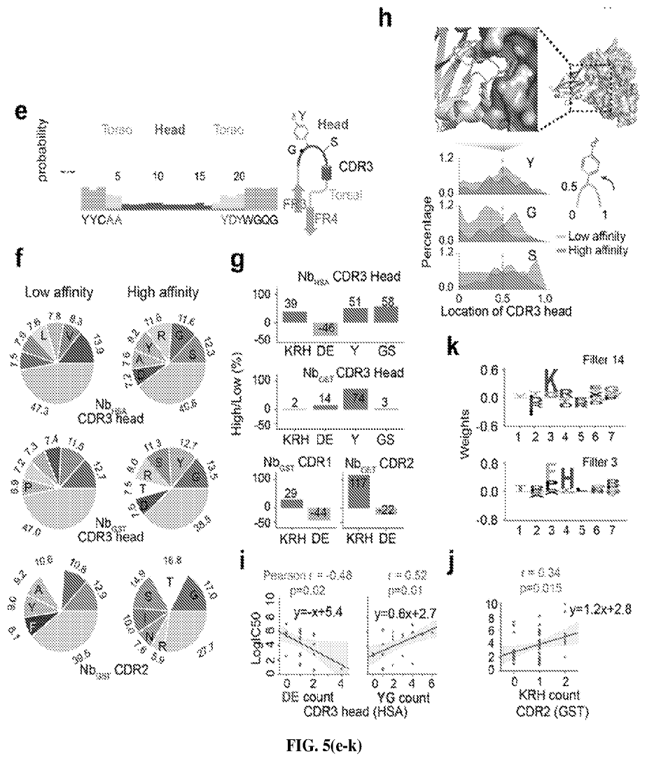

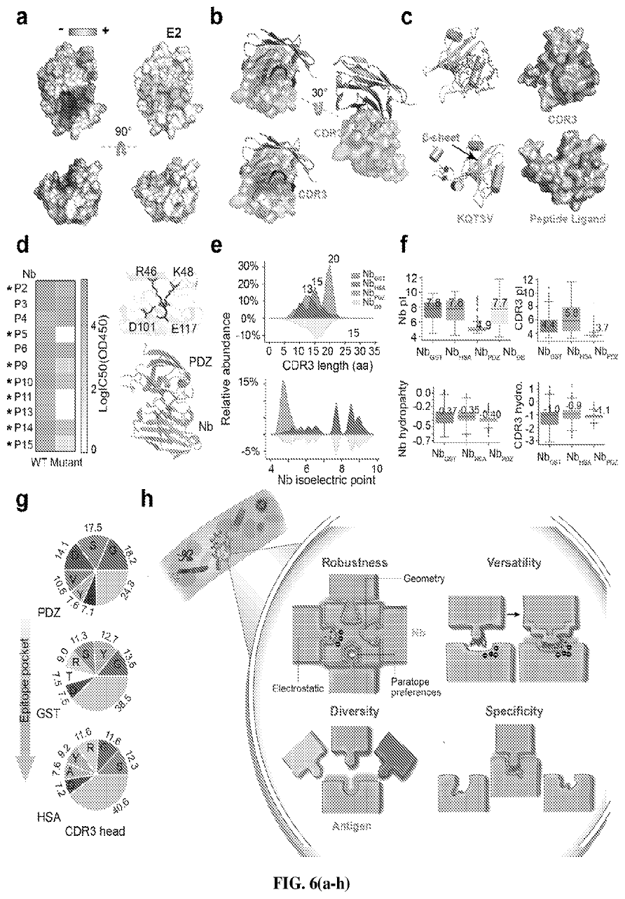

Identification of hundreds of divergent, high-affinity Nb_{CDR3} families for the weakly immunogenic PDZ domain prompted the investigation of the structural basis of such interactions. Two putative epitopes were identified based on docking (FIG. 6A, 13B). E2 can be the major epitope because it has a large positively charged surface (FIG. 6A, 6B) and it is more structured with an α helix and two P-strands. E2 overlapped with the conserved ligand binding sites that are shared among numerous PDZ interacting proteins (Sheng, 2001; Doyle, 1996) (FIG. 6C). Remarkably, Nb_{PDZ} have obtained >100,000-fold higher affinity than natural PDZ ligands (in μM affinity)(Niethammer, 1998) (FIG. 3J). Such high affinity likely was achieved by a long CDR3 loop wrapping around the small and shallow epitope, forming extensive electrostatic and hydrophobic interactions (FIG. 6C, 13A). Modeling results indicated that R46 and K48 of the second β strand in the PDZ epitope formed salt bridges with the corresponding residues in Nb_{PDZ}. A double mutant PDZ (R46E:K48D) was produced and its affinity was evaluated to Nb_{PDZ} by ELISA. The majority (8/11) of Nb_{PDZ} exhibited significantly decreased or no affinity for the mutant, confirming that E2 is indeed the major epitope (FIG. 6D).

There are several other observations on Nb_{PDZ}. First, the distribution of CDR3 loop length formed one major peak with a median of ˜20 aa that pushed the upper limit of its natural distribution (FIG. 6E). Second, Nb_{PDZ} are rather acidic with a median pI of 4.9 (FIG. 6F), which is largely contributed by CDR3 (FIG. 6E, 13F). Third, despite their acidic nature, Nb_{PDZ} did not seem to appreciably alter hydropathy, due to the compensation of hydrophobic residues (FIG. 6G, 13E). Finally, there were significant increases of negatively charged aspartic acid and small glycines and serines, accounting for half of the CDR3 head residues; decrease of bulky tyrosine was also evident compared with high-affinity Nb_{GST} and Nb_{HSA} reflecting the rather shallow pocket of E2 for binding (FIG. 7C, 7E). Collectively, these results demonstrated a remarkable versatility of Nbs for antigen binding.

This study reports the development of a robust platform integrating proteomics, informatics, and structural modeling technologies for analysis of antigen-engaged Nb proteomes. The pipeline enables sensitive and reliable identification of a large repertoire of high-quality Nbs against different challenging antigens. It also enables accurate classification of circulating Nbs based on their physicochemical properties. Thousands of ultrahigh-affinity Nbs were identified by our technologies. Combining computational docking and structural proteomics, the present study have structurally characterized 102,673 antigen-Nb complexes, mapped, and validated the dominant epitopes. This “big data” analysis permits for the first time, global-scale proteomic and structural dissections of the humoral immune response.

These results revealed, at unprecedented depth, the efficiency, specificity, diversity, and versatility of antigen-engaged Nbs that together shape the epic landscapes of camelid antibody immunity (FIG. 6H).

Efficiency: Nbs efficiently utilize both shape and electrostatic complementarity for binding. Specific residues such as charged aspartic acids and arginines, aromatic tyrosines, and small, flexible glycines and serines permit loop flexibility that result in high-affinity Nbs. Intricate and fine-tuned interactions specific for different CDRs were revealed. Moreover, the presence of multiple dominant epitope for Nb binding was confirmed, which can act as a general mechanism for efficiently recognizing pathogens (Akram, A. & Inman, R. D, 2012).

Specificity and Diversity: Thousands of highly divergent Nbs were discovered that evolved to recognize specific HSA surface pockets with some of the most pronounced sequence variations (FIG. 4A) to ensure a specific, effective, and safe immune response.

Versatility: for antigens that tend to evade immune response such as the PDZ, Nbs can drastically alter the size and the physicochemical properties of paratopes to mimic natural ligand binding with outstanding affinity and specificity. The study shows the fascinating rapid evolution of protein-protein interactions.

Nbs are highly potent in viral neutralization and inhibition of enzymatic activities (Lauwereys, 1998; Desmyter, 1996; Acharya, 2013; Arabi, 2017). These findings indicate that these highly robust and efficient camelid HcAbs are evolutionarily advantageous for their survival in both arid natural habitats and aggressive pathogenic challenges, while the driving force(s) behind such an incredible selection and adaptation remains enigmatic (Flajnik, 2011).

These technologies can find broad utility in challenging biomedical applications such as cancer biology, brain research, and virology. These informatics tools for Nb proteomics can be freely available to the research community. The high-quality Nb datasets can serve as a blueprint to study antibody-antigen and can facilitate computational antibody design (Sircar, 2011; Baran, 2017; Chevalier, 2017).

### Example 8. Methods

Animal immunization. Two Llamas were respectively immunized with HSA, and a combination of GST and GST fusion PDZ domain of Mitochondrial outer membrane protein 25 (OMP25) at the primary dose of 1 mg, followed by three consecutive boosts of 0.5 mg every 3 weeks. The bleed and bone marrow aspirates were extracted from the animals 10 days after the last immuno-boost. All the above procedures were performed by Capralogics, Inc. following the IACUC protocol.

mRNA isolation and cDNA preparation. Approximately 1−3×10⁹ peripheral mononuclear cells were isolated from 350 ml immunized blood and 5−9×10⁷ plasma cells were isolated from 30 ml bone marrow aspirates using Ficoll gradient (Sigma). The mRNA was isolated from the respective cells using RNeasy kit (NEB) and was reverse-transcribed into cDNA using Maxima™ H Minus cDNA Synthesis Master Mix (Thermo). Camelid IgG heavy chain cDNA sequences from the variable domain to the CH2 domain were specifically amplified using primers CALL001 (GTCCTGGCTGCTCTTCTACAAGG, SEQ ID NO: 2646) and CH2FORTA4 (CGCCATCAAGGTACCAGTTGA, SEQ ID NO: 2647) (Abrabi, 1997). The VHH genes that lack CH1 domain were separated from conventional IgG and purified (Qiagen) by DNA gel electrophoresis, and were subsequently re-amplified from framework 1 to framework 4 using the 2nd-Forward (ATCTACACTCTITCCCTACACGACGCTCTTCCGATCTNNNNNNNNATGGCT[C/G]A[G/T]GTGCAGCTGGTGGAGTCTGG, SEQ ID NO: 2648, wherein N represents A, T, C or G) and 2nd-Reverse (GTGACTGGAGTTCAGACGTGTGCTCTTCCGATCTNNNNNNNNGGAGACGGTGACCTG GGT, SEQ ID NO: 2649, wherein N represents A, T, C or G). The random 8-mers replacing adaptor sequences were added to aid in cluster identification for Illumina MiSeq. The amplicon of the second PCR (approximately 450-500 bp) was purified using Monarch PCR clean up kit (NEB). The final round of PCR with primer MiSeq-F (AATGATACGGCGACCACCGAGATCTACACTCTITCCCTA, SEQ ID NO: 2650) and MiSeq-R (CAAGCAGAAGACGGCATACGAGATITCTGAATGTGACTGGAGTTCA, SEQ ID NO: 2651) was performed to add P5/P7 adapters with the index before MiSeq sequencing.

Next generation sequencing by Illumina Miseq. Sequencing was performed based on the Illumina MiSeq platform with the 300 bp paired-end model. More than 30 million reads were generated for each database. Read QC tool in FastQC v0.11.8 (www.bioinformatics.babraham.ac.uk/projects/fastqc/) was used for quality check and control of the FASTQ data. Raw Illumina reads were processed by the software tools from the BBMap project (github.com/BiolnfoTools/BBMap/). Duplicated reads and DNA barcode sequences were removed successively before converting the nucleotide sequences into amino acid sequences.

Isolation and biochemical fractionation of V_{H}H antibodies from immunized sera. Approximately 175 ml of plasma was isolated from 350 ml of immunized blood by Ficoll gradient (Sigma). Camelid single-chain V_{H}H antibodies were isolated from the plasma supernatant by a two-step purification procedure using protein G and protein A sepharose beads (Marvelgent), acid-eluted, before neutralized and diluted in 1xPBS buffer to a final concentration of 0.1-0.3 mg/ml. To purify antigen-specific V_{H}H antibodies, the GST or HSA-conjugated CNBr resin was incubated with the VHH mixture for 1 hr at 4C and extensively washed with high salt buffer (1xPBS and 350 mM NaCl) to remove non-specific binders. Specific V_{H}H antibodies were then released from the resin by using one of the following elution conditions: alkaline (1-100 mM NaOH, pH 11, 12 and 13), acidic (0.1 M glycine, pH 3, 2 and 1) or salt elution (1M-4.5 M MgCl₂ in neutral pH buffer). For purification of PDZ-specific VHH, a fusion protein of MBP-PDZ (where the maltose binding protein/MBP was fused to the N terminus of PDZ domain to avoid steric hindrance of the small PDZ after coupling) was produced and was used as the affinity handle. MBP coupled resin was used for control (FIG. 6J). All the eluted V_{H}Hs were neutralized and dialyzed into 1x DPBS separately prior to proteomics analysis.

Proteolysis of Antigen Specific Nbs and Nanoflow Liquid Chromatography coupled to Mass spectrometry (nLC/MS) Analysis. For GST and HSA V_{H}Hs, each elution was processed separately according to the following protocol. For PDZ specific V_{H}Hs, only the most stringent biochemical elutes (i.e., pH 13, pH 1, MgCl₂ 3M and 4.5M) and the respective nonspecific MBP binders (negative controls) from different fractions were pooled for proteolysis. For instance, For PDZ-specific V_{H}Hs that were eluted by pH13 buffer, non-specific MBP binding Nbs were pooled from pH 11, pH12 and pH13 fractions for negative control to improve the stringency of our downstream LC/MS quantification. V_{H}Hs were reduced in 8M urea buffer (with 50 mM Ammonium bicarbonate, 5 mM TCEP and DTr) at 57° C. for 1 hr, and alkylated in the dark with 30 mM Iodoacetamide for 30 mins at room temperature. The alkylated sample was then split into two and in-solution digested using either trypsin or chymotrypsin. For trypsin digestion samples, 1:100 (w/w) trypsin and Lys-C were added and digested at 37° C. overnight, with additional 1:100 trypsin the other morning for 4 hrs at 37° C. water bath. For chymotrypsin digestion samples, 1:50 (w/w) chymotrypsin was added and digested at 37° C. for 4 hrs. After proteolysis, the peptide mixtures were desalted by self-packed stage-tips or Sep-pak C18 columns (Waters) and analyzed with a nano-LC 1200 that is coupled online with a Q Exactive™ HF-X Hybrid Quadrupole Orbitrap™ mass spectrometer (Thermo Fisher). Briefly, desalted Nb peptides were loaded onto an analytical column (C18, 1.6 μm particle size, 100 Å pore size, 75 μm×25 cm; IonOpticks) and eluted using a 90-min liquid chromatography gradient (5% B-7% B, 0-10 min; 7% B-30% B, 10-69 min; 30% B-100% B, 69-77 min; 100% B, 77-82 min; 100% B-5% B, 82 min-82 min 10 sec; 5% B, 82 min 10 sec-90 min; mobile phase A consisted of 0.1% formic acid (FA), and mobile phase B consisted of 0.1% FA in 80% acetonitrile (ACN)). The flow rate was 300 nl/min. The QE HF-X instrument was operated in the data-dependent mode, where the top 12 most abundant ions (mass range 350-2,000, charge state 2-8) were fragmented by high-energy collisional dissociation (HCD). The target resolution was 120,000 for MS and 7,500 for tandem MS (MS/MS) analyses. The quadrupole isolation window was 1.6 Th and the maximum injection time for MS/MS was set at 80 ms.

Nb DNA synthesis and cloning. Nb genes were codon-optimized for expression in Escherichia coli and the nucleotides were in vitro synthesized (Synbiotech). After verification by Sanger sequencing, the Nb genes were cloned into a pET-21b (+) vector at BamHI and XhoI (for GST Nbs), or EcoRI and NotI restriction sites (for HSA and PDZ Nbs).

Purification of recombinant Proteins. DNA constructs were transformed into BL21 (DE3) competent cells according to manufacturer's instructions and plated on Agar with 50 pg/ml ampicillin at 37° C. overnight. A single colony was inoculated in LB medium with ampicillin for overnight culture at 37° C. The culture was then inoculated at 1:100 (v/v) in fresh LB medium and shaked at 37° C. until the O.D.600 nm reached 0.4-0.6. GST, GST-PDZ and Nbs were induced with 0.5 mM of IPTG while MBP and MBP-PDZ were induced with 0.1 mM of IPTG. The inductions were performed at 16° C. overnight. Cells were then harvested, briefly sonicated and lysed on ice with a lysis buffer (1xPBS, 150 mM NaCl, 0.2% TX-100 with protease inhibitor). After lysis, soluble protein extract was collected at 15,000×g for 10 mins. GST and GST-PDZ were purified using GSH resin and eluted by glutathione. MBP (maltose binding protein) and MBP-PDZ fusion protein were purified by using Amylose resin and were eluted by maltose according to the manufacturer's instructions. Nbs were purified by His-Cobalt resin and were eluted using imidazole. The eluted proteins were subsequently dialyzed in the dialysis buffer (e.g., 1x DPBS, pH 7.4) and stored at −80° C. before use.

Nb immunoprecipitation assay. After Nb induction and cell lysis, the cell lysates were run on SDS-PAGE to estimate Nb expression levels. Recombinant Nbs in the cell lysis were diluted in 1x DPBS (pH 7.4) to a final concentration of ˜5 μM (for GST Nbs) and ˜50 nM (for PDZ Nbs). To test the specific interactions of Nbs with antigens, different antigens were coupled to the CNBr resin. Inactivated or MBP-conjugated CNBr resin was used for control. Antigen coupled resins or control resins were incubated with Nb lysates at 4° C. for 30 mins. The resins were then washed three times with a washing buffer (1x DPBS with 150 mM NaCl and 0.05% Tween 20) to remove nonspecific bindings. Specific antigen bound Nbs were then eluted from the resins by the hot LDS buffer containing 20 mM DTT and ran on SDS-PAGE. The intensities of Nbs on the gel were compared between antigen specific signals and control signals to derive the false positive binding.

ELISA (enzyme-linked immunosorbent assay). Indirect ELISA was carried out to evaluate the camelid immune response of an antigen and to quantify the relative affinities of antigen-specific Nbs. An antigen was coated onto a 96-well ELISA plate (R&D system) at an amount of approximately 1-10 ng per well in a coating buffer (15 mM sodium carbonate, 35 mM sodium bicarbonate, pH 9.6) overnight at 4° C. The well surface was then blocked with a blocking buffer (DPBS, 0.05% Tween 20, 5% milk) at room temperature for 2 hours. To test an immune response, the immunized serum was serially 5-fold diluted in the blocking buffer. The diluted sera were incubated with the antigen coated wells at room temperature for 2 hours. HRP-conjugated secondary antibodies against llama Fc (Bethyl) were diluted 1:10,000 in the blocking buffer and incubated with each well for 1 hour at room temperature. For Nb affinity tests, scramble Nbs that do not bind the antigen of interest were used for negative controls. Nbs of both specific binders for test and scramble negative controls were serially 10-fold diluted from 10 μM to 1 μM in the blocking buffer. HRP-conjugated secondary antibodies against His-tag (Genscript) or T7-tag (Thermo) were diluted 1:5,000 or 1:10,000 in the blocking buffer and incubated for 1 hour at room temperature. Three washes with 1x PBST (DPBS, 0.05% Tween 20) were carried out to remove nonspecific absorbance between incubations. After the final wash, the samples were further incubated under dark with freshly prepared w3,3′,5,5′-Tetramethylbenzidine (TMB) substrate for 10 mins at room temperature to develop the signals. After the STOP solution (R&D system), the plates were read at multiple wavelengths (450 nm and 550 nm) on a plate reader (Multiskan GO, Thermo Fisher). A false positive Nb binder was defined if any of the following two criteria was met: i) the ELISA signal can only be detected at a concentration of 10 μM and was under detected at 1 μM concentration. ii) At 1 μM concentration, a pronounced signal decrease (by more than 10-fold) was detected compared to the signal at 10 μM, while there were no signals can be detected at lower concentrations. The raw data was processed by Prism 7 (GraphPad) to fit into a 4PL curve and to calculate logIC50.

Nb affinity measurement by SPR. Surface plasmon resonance (SPR, Biacore 3000 system, GE Healthcare) was used to measure Nb affinities. Antigen proteins immobilized on the activated CM5 sensor-chip by the following steps. Protein analytes were diluted to 10-30 μg/ml in 10 mM sodium acetate, pH 4.5, and were injected into the SPR system at 5 μl/min for 420 s. The surface of the sensor was then blocked by 1 M ethanolamine-HCl (pH 8.5). For each Nb analyte, a series of dilution (spanning three orders of magnitude) was injected in HBS-EP+ running buffer (GE-Healthcare) containing 2 mM DTT, at a flow rate of 20-30 μl/min for 120-180 s, followed by a dissociation time of 5-20 mins based on dissociation rate. Between each injection, the sensor chip surface was regenerated with the low pH buffer containing 10 mM glycine-HCl (pH 1.5-2.5), or high pH buffer of 20-40 mM NaOH (pH 12-13). The regeneration was performed with a flow rate of 40-50 μl/min for 30 s. The measurements were duplicated and only highly reproducible data was used for analysis. Binding sensorgrams for each Nb were processed and analyzed using BIAevaluation by fitting with 1:1 Langmuir model or 1:1 Langmuir model with mass transfer.

Cross-linking and mass spectrometric analysis of antigen-nanobody complex. Different Nbs were incubated with the antigen of interest with equal molarity in an amine-free buffer (such as 1x DPBS with 2 mM DTT) at 4° C. for 1-2 hours before cross-linking. The amine-specific disuccinimidyl suberate (DSS) or heterobifunctional linker 1-ethyl-3-(3-dimethylaminopropyl) carbodiimide hydrochloride (EDC) was added to the antigen-Nb complex at 1 mM or 2 mM final concentration, respectively. For DSS cross-linking, the reaction was performed at 23° C. for 25 mins with constant agitation. For EDC cross-linking, the reaction was performed at 23° C. for 60 mins. The reactions were quenched by 50 mM Tris-HCl (pH 8.0) for 10 mins at room temperature. After protein reduction and alkylation, the cross-linked samples were separated by a 4-12% SDS-PAGE gel (NuPAGE, Thermo Fisher). The regions corresponding to the cross-linked species were cut and in-gel digested with trypsin and Lys-C as previously described (Shi, 2014; Shi, 2015). After proteolysis, the peptide mixtures were desalted and analyzed with a nano-LC 1200 (Thermo Fisher) coupled to a Q Exactive™ HF-X Hybrid Quadrupole-Orbitrap™ mass spectrometer (Thermo Fisher). The cross-linked peptides were loaded onto a picochip column (C18, 3 μm particle size, 300 Å pore size, 50 μm×10.5 cm; New Objective) and eluted using a 60 min LC gradient: 5% B-8% B, 0-5 min; 8% B-32% B, 5-45 min; 32% B-100% B, 45-49 min; 100% B, 49-54 min; 100% B-5% B, 54 min-54 min 10 sec; 5% B, 54 min 10 sec-60 min 10 sec; mobile phase A consisted of 0.1% formic acid (FA), and mobile phase B consisted of 0.1% FA in 80% acetonitrile. The QE HF-X instrument was operated in the data-dependent mode, where the top 8 most abundant ions (mass range 380-2,000, charge state 3-7) were fragmented by high-energy collisional dissociation (normalized collision energy 27). The target resolution was 120,000 for MS and 15,000 for MS/MS analyses. The quadrupole isolation window was 1.8 Th and the maximum injection time for MS/MS was set at 120 ms. After MS analysis, the data was searched by pLink2 for the identification of cross-linked peptides (Chen, 2019). The mass accuracy was specified as 10 and 20 p.p.m. for MS and MS/MS, respectively. Other search parameters included cysteine carbamidomethylation as a fixed modification and methionine oxidation as a variable modification. A maximum of three trypsin missed-cleavage sites was allowed. The initial search results were obtained using the default 5% false discovery rate, estimated using a target-decoy search strategy. The crosslink spectra were then manually checked to remove false-positive identifications essentially as previously described (Shi, 2014; Kim, 2018; Shi, 2015).

Site-directed mutagenesis. Mammalian expression plasmid of HSA was obtained from Addgene. E400R point mutation was introduced to the HSA sequence by the Q5 site-directed mutagenesis kit (NEB) using the primer HSA-F (GGTGTTCGACCGGTTCAAGCCTCTGG, SEQ ID NO: 2652) and HSA-R (TTGGCGTAGCACTCGTGA, SEQ ID NO: 2653). After sequence verification by Sanger Sequencing, plasmids bearing wild type HSA and the mutant were transfected to HeLa cells using Lipofectamine 3000 transfection kit (Thermo) and Opti-MEM (Gibco) according to the manufacturer's protocol. The cells were cultured overnight before change of medium to DMEM without FBS supplements to remove BSA. After a 48 h culture at 37° C., 5% CO₂, the media expressing HSA were collected and stored at −20° C. The media were analyzed by SDS-PAGE and Western Blotting to confirm protein expression.

The PDZ domain (in the pGEX6p-1 vector) was obtained from the General Biosystems. A double point mutant of PDZ (i.e., R46E: K48D) was introduced by the Q5 Site-directed mutagenesis kit using specific primers of PDZ-F (TGATGAAAATGGCGCAGCCGCC, SEQ ID NO: 2654) and PDZ-R (ATITCACTCACATAGATACCACTATCATTACTAACATAC, SEQ ID NO: 2655). After verification by Sanger Sequencing, the mutant vector was transformed into BL21(DE3) cells for expression. The GST fusion PDZ mutant protein was purified by GSH resin as previously described.

Fluorescence Microscopy. COS-7 cells were plated onto the glass bottom dish at an initial confluence of 60-70% and cultured overnight to let the cells attach to the dish. Cells were with MitoTracker Orange CMTMRos (1:4000) at 37° C. for 30 minutes, washed once with PBS and fixed with pre-cold methanol/ethanol (1:1) for 10 minutes. After being washed with PBS, the cells were blocked with 5% BSA for 1 hour. Alexa Fluor™ 647-conjugated Nb (1:100) was then added to the cells, incubated for 15 minutes at room temperature. Two-color wide-field fluorescence images were acquired using our custom-built system on an Olympus IX71 inverted microscope frame with 561 nm and 642 nm excitation lasers (MPB Communications, Pointe-Claire, Quebec, Canada) and a 100×oil immersion objective (NA=1.4, UPLSAPO 100XO; Olympus).

Text-based CDR (complementarity-determining region) Annotation. The CDR annotation method was modified from (Fridy, 2014). [*] denotes any residue.

CDR1 annotation: The short sequence motif “SC” was first searched, which is localized between the residue 20-residue 26 of a Nb sequence. The start of a CDR1 sequence is defined as the 5th residue followed by the “SC” motif. Once the first residue is identified, we then look for another sequence motif “W[*]R” which is localized between Nb residue 32-residue 40, and define the end of the CDR1 sequence as the first residue preceding the “W[*]R” motif.

CDR2 annotation: The start of a CDR2 sequence is defined as the 14th residue followed by the “W[*]R” motif. Once the first residue is identified, motif “RF” which is localized between Nb residue 63-residue 72 was then identified, and the end of the CDR2 sequence as the 8th residue preceding the “RF” motif was defined.

CDR3 annotation: The motif of “Y[*]C” or “YY[*]” was first searched, which is localized between Nb residue 90-residue 105. The start of a CDR3 sequence is defined as the 3rd residue followed by the “Y[*]C” or “YY[*]” motif. Once the first residue of a CDR3 was identified, either one of the following sequence motifs (“WG[*]G”, “WGQ[*]”, “W[*]Q[*]”, “[*]GQG”, “[*][*]GQ” and “WG[*][*]”) was then used to locate the end of the CDR3. These motifs are located within the last 14 residues of the C terminal Nb sequence. CDR3 ends at 1 residue ahead of the sequence motif. More information can be found in the Augur Llama scripts.
The cleavage rules for in-silico digestion of Nbs by different proteases:
Trypsin: C-terminal to K/R, not followed by P
Chymotrypsin: C-terminal to W/F/UY, not followed by P
GluC: C-terminal to D/E, not followed by P
AspN: N-terminal to D
LysC: C-terminal to K

Sequence alignment of Nb database: Nb sequences were aligned using the software ANARCI (Dunbar, J. & Deane, C. M, 2016). Three CDRs (CDR 1-CDR3) and four Framework sequences (FR1-FR4) were annotated according to IMGT numbering scheme (Lefranc, 2003). Alignments below the threshold e-value of 100 were removed and the remaining sequences were plotted by WebLogo (Crooks, 2004).

In-silico digestion of Nb database by different proteases and analysis of Nb CDR3 mapping. A high-quality database containing approximately 0.5 million unique Nb sequences was in-silico digested using different enzymes including trypsin, chymotrypsin, LysC, GluC, and AspN according to the above cleavage rules. CDR3 containing peptides were obtained to calculate the sequence coverages. The CDR3 coverages were then summed to generate FIGS. 1D & 7B. The CDR3 peptide length distributions (by trypsin and chymotrypsin) were plotted to generate FIG. 1E.

Simulation of trypsin and chymotrypsin-aided MS mapping of Nbs. 10,000 Nb sequences with unique CDR3 fingerprint sequences were randomly selected from the database. The selected Nbs were then in-silico digested by either trypsin or chymotrypsin (with no-miscleavage sites allowed) to generate CDR3 peptides. The following criteria were applied to these peptides to better simulate Nb identifications by MS: 1) peptides of favorable sizes for bottom-up proteomics (between 850-3,000 Da) were first selected. 2) Peptides containing the highly conserved C-terminal FR4 motif of WGQGQVTS were further discarded. Based on our observations, such peptides are often dominated by C terminal y ion fragmentations, while having poorly fragmented ions on the CDR3 sequence which are essential for unambiguous CDR3 peptide identifications. 3) CDR3 peptides with limited Nb fingerprint information (containing less than 30% CDR3 sequence coverage) were removed. As a result, 2,111 unique tryptic peptides and 5,154 unique chymotryptic peptides were obtained. These peptides were then used to map Nb proteins. After protein assembly, only Nb identifications with sufficiently high CDR3 fingerprint sequence coverages (>60%) were used to generate the venn diagram in FIG. 1F.

Phylogenetic analysis of Nb CDR3 sequences. Phylogenetic trees were generated by Clustal Omega (Sievers, 2014) with the input of unique Nb CDR3 sequences and the additional flanking sequences (i.e., YYCAA to the N-term and WGQG to the C-term of CDR3 sequences) to assist alignments. The data was plotted by ITo1 (Interactive Tree of Life) (Letunic, I. & Bork, P, 2007). Isoelectric points and hydrophobicities of Nb CDR3s were calculated using the BioPython library. Sequence alignments were visualized by Jalview (Waterhouse, 2009).

Evaluation of the reproducibility of Nb peptide quantification. Shared peptide identifications among different LC runs were used to evaluate the reproducibility of the label-free quantification method. For a typical 90 min LC gradient, the peptide peak width or full width at half maximum (FWHM) in general was less than 5s. The differences of peptide retention time among different LC runs were calculated to generate the kernel density estimation plots in FIG. 3B. Peptide retention times from different LC runs were used to calculate pearson correlation and were plotted in FIG. 9B.

Sequence alignment and analysis of HSA and Llama serum albumin. Llama (Camelus Ferus) serum albumin sequence was fetched and aligned with HSA by tblastn (NCBI). The isoelectric point (pI) and hydropathy values for individual amino acids were obtained online from (www.peptide2.com/N_peptide_hydrophobicity_hydrophilicity.php). These values were normalized between 0 to 1.0 and the sequence variations between the two albumins were calculated for each aligned position (the pairwise differences of pI and hydropathy). For a specific aligned residue position, a value of 0 indicates identical residues were found between the two sequences, while 1.0 indicates the largest sequence variation, such as a charge reversion from the negatively charged residue glutamic acid 400 for HSA to the positively charged residue arginine at the corresponding aligned position for camelid albumin. A value of 0.5 was assigned at the position where an insertion or deletion of amino acid was identified. Sequence variations of both pI and hydropathy between HSA and Llama serum albumin were thus plotted. The plots were further smoothed by a gaussian function to generate FIG. 4A.

Analysis of relative abundance of amino acids on Nb CDRs. The amino acid frequencies at each CDR (including CDR1, CDR2 and CDR3 head) were calculated and normalized to generate the bar plots and the pie plots in FIGS. 6, 7, 12 and 13. CDR3 head sequences were obtained by removing the semi-conserved C terminal four residues of CDR3s. The CDR residue frequencies of both high-affinity and low-affinity Nbs were normalized based on the sum of the CDR residues of each affinity group.

Analysis of amino acid positions on CDR3 heads. The relative position of a residue on a CDR3 head was calculated where a value of 0 indicates the very N terminus of a CDR3 head while 1.0 indicates the last residue. The CDR3 head sequences were then sliced into 20 bins with a bin width of 0.05. Within each bin, the occurrence of a specific type of amino acid (such as tyrosine, glycine, or serine) was counted and normalized to the sum of residues on CDR3 heads. The distributions of different amino acids including their relative positions and abundances were plotted in FIGS. 5H and 12G.

Proteomics database search of Nb peptide candidates. Raw MS data was searched by Sequest HT embedded in the Proteome Discoverer 2.1 (Thermo Fisher) against an in-house generated Nb sequence database using the standard target-decoy strategy for FDR estimation. The mass accuracy was specified as 10 ppm and 0.02 Da for MS1 and MS2, respectively. Other search parameters included cysteine carbamidomethylation as a fixed modification and methionine oxidation as a variable modification. A maximum of one or two missed-cleavage sites was allowed for trypsin and chymotrypsin-processed samples respectively. The initial search results were filtered by percolator with the FDR of 0.01 (strict) based on the q-value (Kall, 2007). After database search, the peptide-spectrum-matches (PSMs) were exported, processed and analyzed by Augur Llama with following steps:

a. Nanobody Identification

i) Quality Assessment of CDR3 Fingerprints

Peptide candidates were first annotated as either CDR or FR peptides. To confidently identify CDR3 fingerprint peptides, we implemented a filter/algorithm requiring sufficient coverage of high-resolution CDR3 fragment ions in the PSMs (See illustration in FIG. 8B). The filter was evaluated using a target sequence database containing approximately 0.5 million unique Nb sequences and a non-overlapping decoy database of similar size. Target and decoy Nb sequence databases herein used were obtained from different llamas. Any peptide identification from the decoy database was considered as a false positive. The FDR was defined based on the % of peptide identifications from the decoy database compared with those from the target database. CDR3 length was also considered to enable development of a sensitive CDR3 peptide filter. The CDR3 fragmentation coverage was defined as the percentage of the CDR3 residues that were matched by fragment ions (either b ions or y ions) within the mass accuracy window. Spectra of the same peptide were combined for assessment. Only CDR3 peptides that passed this filter (5% FDR) were selected for the downstream Nb assembly.

ii) Nanobody Sequence Assembly

CDR peptides including the confident CDR3 peptides were used for Nb protein assemblies. Two additional criteria must be matched before a Nb can be identified. These include: 1) both CDR1 and CDR2 peptides must be available for a Nb assembly. 2) for any Nb identification, a minimum of 50% combined CDR coverage was mandated.

b. Quantification and Classification of Antigen-Specific Nb Repertoires

MS raw data was accessed by MSFileReader 3.1 SP4(ThermoFisher), and a python library of pymsfilereader (github.com/frallain/pymsfilereader). Reliable CDR3 peptides that passed the quality filter were quantified by label-free LC/MS.

i) CDR3 Peptide Quantification

To enable accurate label-free quantification of CDR3 peptide identification across different LC runs, different retention time windows for peptide peak extraction were specified. For peptides that can be directly identified by the search engine based on the MS/MS spectra, a small quantification window of +/−0.5 minutes retention time (RT) shift was used for peak extractions. For peptides that were not directly identified from a particular LC run (due to the complexity of peptides and stochastic ion sampling), their RTs were predicted based on the RT of the adjacent LC and were adjusted using the median RT difference of the commonly identified peptides between the two LC runs. In this case, a relaxed RT window of +/−2.0 minutes (for a typical 90 min LC gradient), in which approximately 95% of all the identified peptides can be matched between the two LC runs, was applied to facilitate extraction of the peptide peaks. Both m/z and z of a peptide were used for peak extractions with a mass accuracy window of +/−10 ppm. The peptide peaks were extracted and smoothed using a Gaussian function. Their AUCs (area under the curve) were calculated and AUCs from the replicated LC runs were averaged to infer the CDR3 peptide intensities.

ii) Classifications of Nbs

To enable accurate classifications e.g., based on Nb affinities, relative ion intensities (AUCs) of the CDR3 fingerprint peptides among three different biochemically fractionated Nb samples (F), F2 and F3) were quantified as I1, I2 and I3. Based on the quantification results, CDR3 peptides were arbitrarily classified into three clusters (C1, C2, and C3) using the following criteria:
1) For C3 (high-affinity) cluster: I3>I1+I2 (indicating Nbs were more specific to F3)
2) For C2 (mediocre-affinity) cluster: I2>I1+I3 (indicating Nbs were more specific to F2)
3) For C1 (low-affinity) cluster:

I1>I2+I3 (indicating Nbs were either more specific to F) or likely nonspecific binders), alternatively, if I1<I2+I3 and I2<I1+I3 and I3<I1+I2, these Nb identifications were likely nonspecifically identified and were grouped into C1 as well. See illustration in FIG. 8C.

The above method was used to classify HSA and GST Nbs. Some modifications were made for quantification and characterization of high-affinity PDZ Nbs. Specifically, an additional control of MBP interacting Nbs “F_control” (ion intensity of I_control) was included for quantification. High-affinity cluster Nbs (represented by their unique CDR3 peptides) were defined when the sum intensities of I2 and I3 for a Nb CDR3 peptide were 20 fold higher than I_control(i.e. 20*I_control <I2+I3). For Nbs where more than one unique CDR3 peptide was used for quantification, classification results among different CDR3 peptides from the same Nb must be consistent; otherwise, they were removed before the final results were reported.

Heatmap analysis of the relative intensities of CDR3 peptides. The identified CDR3 peptides were quantified based on their relative MS1 ion intensities and were subsequently clustered using scripts in Augur Llama. Z-scores were calculated based on the relative ion intensities and were used to generate a heatmap in FIG. 3A for visualization.

Structural modeling of antigen-Nb complexes. Structural models for Nbs were obtained using a multi-template comparative modeling protocol of MODELLER (Webb, B. & Sali, A, 2014). Next, we refine the CDR3 loop and select the top 5 scoring loop conformations for the downstream docking. Each Nb model is then docked to the respective antigen by an antibody-antigen docking protocol of PatchDock software that focuses the search to the CDRs (Schneidman-Duhovny, 2005). The models are then re-scored by a statistical potential SOAP (Dong, 2013). The antigen interface residues (distance <XÅ from Nb atoms) among the 10 best scoring models according to the SOAP score were used to determine the epitopes. Once the epitopes were defined, we clustered Nbs based on the epitope similarity using k-means clustering. The clusters reveal the most immunogenic surface patches on the antigens. Antigen-Nb complexes with CXMS data were modeled by distance-restrained based PatchDock protocol that optimizes restraints satisfaction (Schneidman-Duhovny, 2020; Russel, 2012). A restraint was considered satisfied if the Ca-Ca distance between the cross-linked residues was within 25 Å and 20 Å for DSS and EDC cross-linkers, respectively (Shi, 2014; Fernandez-Martinez, 2016). In the case of ambiguous restraints, such as the GST dimer, it is required that one of the cross-links is satisfied.

Machine learning analysis of Nb repertoires. A deep neural network was trained to distinguish between low- and high-affinity Nbs that were characterized by the accurate high-pH fractionation method and quantitative proteomics. This model consists of one convolutional layer with batch normalization and ReLU activation function, followed by a max pooling layer ending with a fully connected layer to integrate the features extracted into the logits layer that leads to the classifier prediction. The convolutional layer consists of 20 1D filters, representing local receptive fields with window size of 7 amino acids, long enough to capture the relevant CDRs and short enough to avoid data overfitting. During the forward pass, each filter slides along the protein sequence with a fixed stride performing an elementwise multiplication with the current sequence window, followed by summing it up to generate a filter response. The classification accuracy of the model was 92%.

To understand the physicochemical features learned by the network for distinguishing low- and high-affinity binders, the activation path was calculated through the network back from the prediction to the activated filter. Similar to the backpropagation algorithm, backward was iterated from the last two layers of fully connected network, extracting for each sequence the output signal and looking for the highest peaks which contribute the most weight to the classification. In the same way, upstream the contribution of each filter to those peaks was calculated. In addition, filter activity in CDRs was analyzed to extract region-specific dominant filters. This process of network interpretation results in a unique contribution per filter per sequence. Each filter is activated along the sequence downsampled in the max pooling layer. For each filter, its highest peak was then picked leading to classification. Finally, the most contributing filters per sequence was determined and there also we got an interesting filter out with more than 30% contribution in those regions of interest.

### Computer Implemented Methods

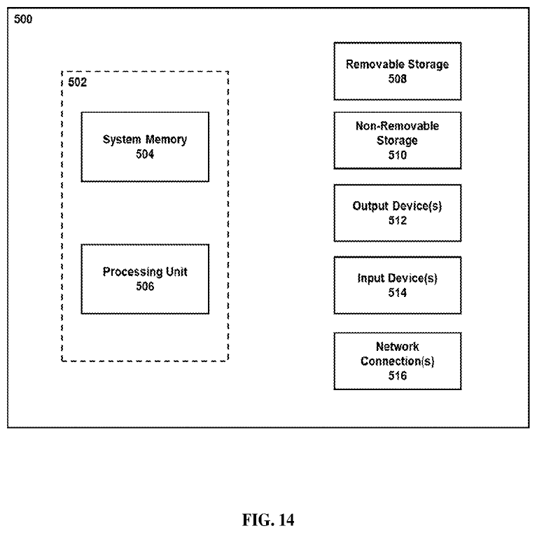

It should be appreciated that the logical operations described herein with respect to the various figures may be implemented (1) as a sequence of computer implemented acts or program modules (i.e., software) running on a computing device (e.g., the computing device described in FIG. 14), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 14, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 14 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

As noted above, logical operations described herein, for example logical operations as described in Example 8, can be implemented with hardware, software or, where appropriate, with a combination thereof. For example, the logical operations can be implemented using one or more computing devices such as computing device 500 of FIG. 14. Logical operations described in Example 8 include, but are not limited to, methods for determining antigen affinity of nanobody peptide sequences, methods for training deep learning models, and deep learning-based methods for inferring antigen affinity of nanobody peptide sequences. These operations are described in detail above.

In some embodiments, a computer-implemented method includes:

receiving a nanobody peptide sequence;

identifying a plurality of CDR regions of the nanobody peptide sequence, the CDR regions including CDR3 regions;

applying a fragmentation filter to discard one or more false positive CDR3 regions of the nanobody peptide sequence;

quantifying an abundance of one or more non-discarded CDR3 regions of the nanobody peptide sequence; and

inferring an antigen affinity based on the quantified abundance of the one or more non-discarded CDR3 regions of the nanobody peptide sequence.

In some embodiments, a method for training a deep learning model includes:

creating a dataset that comprises a plurality of nanobody peptide sequences and corresponding antigen-affinity labels; and

training, using the dataset, a deep learning model to classify nanobody peptide sequences having low antigen affinity and nanobody peptide sequences having high antigen affinity.

In some embodiments, a method for determining antigen affinity of nanobody peptide sequences includes:

receiving a nanobody peptide sequence;

inputting the nanobody peptide sequence into a trained deep learning model; and

classifying, using the trained deep learning model, the nanobody peptide sequence as having low antigen affinity or high antigen affinity.

TABLE 1
Summary of GST Nbs and their biophysical and physiochemical properties

| | | | | | | | | Binder | | | | | |
|---|---|---|---|---|---|---|---|---|---|---|---|---|---|
| | | | | | | | | by | | | | | |
| | | | | | | | | Beads- | ELISA | | | | |
| | | | | | | | | binding | affinity | | | | |
| | | | SEQ ID | Salt | LowpH | HighpH | | Assay | (LogIC50 | SPR ka | SPR kd | SPR KD | Cross- |
| ID | Enzyme | Protein Sequence | NO | Trend | Trend | Trend | Soluble | (FIG. S3C) | (oD450nm)) | (1/Ms) | (1/s) | (M) | linker |
| G1 | Chymo | MASMTGGQQMGRNSAQ | SEQ ID | 0 | 0 | 2 | Yes | / | 2.93 | / | | | / |
| | | VQLVESGGGLVQAGGSL | NO: 1 | | | | | | | | | | |
| | | RLSCAAPGSTFSTNIIAW | | | | | | | | | | | |
| | | YRQPPGKQRELVAAIGG | | | | | | | | | | | |
| | | PGSTNYADSVKGRFTISR | | | | | | | | | | | |
| | | DNAKNTGYLQMKSLKP | | | | | | | | | | | |
| | | DDTAVYYCNMVTQRGN | | | | | | | | | | | |
| | | EYWGQGTQVTVSSEPKT | | | | | | | | | | | |
| | | PKGGCGGGLEHHHHHH | | | | | | | | | | | |
| G2 | Trypsin/ | MASMTGGQQMGRNSAE | SEQ ID | / | 1 | 0 | Yes | / | 2.667 | 1.02E+03 | 2.04E−03 | 2.00E−06 | / |
| | Chymo | VQLVESGGDLVQAGGSL | NO: 2 | | | | | | | | | | |
| | | RLSCSASGNIFKINDMG | | | | | | | | | | | |
| | | WYRQAPGKQRELVARIS | | | | | | | | | | | |
| | | SSGNTNYADSVKGRFTIS | | | | | | | | | | | |
| | | RDNGKNTVYLQMNRVK | | | | | | | | | | | |
| | | PEDTAVYYCNADVQVS | | | | | | | | | | | |
| | | RNYEYEYWGQGTQVTV | | | | | | | | | | | |
| | | SSEPKTPKGGCGGGLEH | | | | | | | | | | | |
| | | HHHHH | | | | | | | | | | | |
| G3 | Trypsin | MASMTGGQQMGRNSAQ | SEQ ID | / | / | 0 | Yes | Yes | / | / | | | / |
| | | VQLVESGGGLVQAGGSL | NO: 3 | | | | | | | | | | |
| | | RLSCAASAGTFSTYAISW | | | | | | | | | | | |
| | | FRQAPGKERDFVAAINRI | | | | | | | | | | | |
| | | SRSAYSPYYADSVKGRF | | | | | | | | | | | |
| | | TISEDNAKNTVNLQMNS | | | | | | | | | | | |
| | | LKPEDTAVYYCAAGSIF | | | | | | | | | | | |
| | | HTDVRYYAYWARGPRS | | | | | | | | | | | |
| | | PSSEPKTPKGGCGGGLE | | | | | | | | | | | |
| | | HHHHHH | | | | | | | | | | | |
| G4 | Trypsin/ | MASMTGGQQMGRNSAE | SEQ ID | / | 0 | / | Yes | Yes | / | / | | | / |
| | Chymo | VQLVESGGGLVQPGGSL | NO: 4 | | | | | | | | | | |
| | | RLSCSASGRTLDSYGIG | | | | | | | | | | | |
| | | WFRQAPGKEREEVSCISS | | | | | | | | | | | |
| | | SGGNADYADSVMGRFTI | | | | | | | | | | | |
| | | SRDNAKNTVYLHMNNL | | | | | | | | | | | |
| | | RPEDTAVYYCAAIAGLC | | | | | | | | | | | |
| | | ALHYTDYKVVVIPGSWG | | | | | | | | | | | |
| | | QGTQVTVSSEPKTPKGG | | | | | | | | | | | |
| | | CGGGLEHHHHHH | | | | | | | | | | | |
| G5 | Trypsin | MASMTGGQQMGRNSAE | SEQ ID | / | 0 | 0 | Yes | / | 0, No | / | | | / |
| | | VQLVESGGGVVQPGGSL | NO: 5 | | | | | | binding | | | | |
| | | TLSCAASGFAFRNYAMS | | | | | | | | | | | |
| | | WVRQAPGKGPEWVSQI | | | | | | | | | | | |
| | | NGRGGYTSYADSVKGRF | | | | | | | | | | | |
| | | TISRDNTKNTLYLQMNN | | | | | | | | | | | |
| | | LKPDDTAVYYCAKDPTQ | | | | | | | | | | | |
| | | LRWIPVPNYILGSTKGQG | | | | | | | | | | | |
| | | TQVTVSSEPKTPKGGCG | | | | | | | | | | | |
| | | GGLEHHHHHH | | | | | | | | | | | |
| G6 | Chymo | MASMTGGQQMGRNSAQ | SEQ ID | / | / | 0 | Yes | / | 0, No | / | | | / |
| | | VQLVESGGGLVQAGGSL | NO: 6 | | | | | | binding | | | | |
| | | RLSCAASGRTISSYAMG | | | | | | | | | | | |
| | | WFRQAPGKERELVARIT | | | | | | | | | | | |
| | | SSAGSTYYADSVKGRFTI | | | | | | | | | | | |
| | | SRDNAKNTMYLQMNSL | | | | | | | | | | | |
| | | KPEDTAVYYCAVEIVRA | | | | | | | | | | | |
| | | QYDYWGQGTQVTVSSE | | | | | | | | | | | |
| | | PKTPKGGCGGGLEHHHH | | | | | | | | | | | |
| | | HH | | | | | | | | | | | |
| G7 | Chymo | MASMTGGQQMGRNSAQ | SEQ ID | / | / | 0 | Yes | Yes | 1.885 | 3.02E+03 | 8.09E−04 | 2.68E−07 | / |
| | | VQLVESGGGLVQPGDSL | NO: 7 | | | | | | | | | | |
| | | RLSCAVSGQYVNMAAM | | | | | | | | | | | |
| | | GWFRQAPGKEREFVAGI | | | | | | | | | | | |
| | | SWSDDTDIADSVKGRFTI | | | | | | | | | | | |
| | | SRDHGKNTVDLQMNSL | | | | | | | | | | | |
| | | KPEDTGVYLCAGRFRRL | | | | | | | | | | | |
| | | AKDFGEYDYWGQGTQV | | | | | | | | | | | |
| | | TVSSEPKTPKGGCGGGL | | | | | | | | | | | |
| | | EHHHHHH | | | | | | | | | | | |
| G8 | Trypsin | MASMTGGQQMGRNSAQ | SEQ ID | 1 | 0 | 1 | Yes | / | 4.658 | / | | | / |
| | | VQLVESGGGLVQAGGSL | NO: 8 | | | | | | | | | | |
| | | RLSCAASGITSSIASMGW | | | | | | | | | | | |
| | | FRQAPGKEEEFVARIRW | | | | | | | | | | | |
| | | NTDNTYYADSVKGRFTI | | | | | | | | | | | |
| | | SRDNAQNTVYLQMNRL | | | | | | | | | | | |
| | | KPEDTAVYYCVARRGW | | | | | | | | | | | |
| | | SDLLYDYRGQGTQVTVS | | | | | | | | | | | |
| | | SEPKTPKGGCGGGLEHH | | | | | | | | | | | |
| | | HHHH | | | | | | | | | | | |
| G9 | Trypsin | MASMTGGQQMGRNSAD | SEQ ID | 1 | / | 2 | Yes | Yes | 8.05 | 2.00E+06 | 3.53E−04 | 1.77E−10 | DSS |
| | | VQLVESGGGLVQPGGSL | NO: 9 | | | | | | | | | | |
| | | RLSCAASGLTLDNYDMA | | | | | | | | | | | |
| | | WFRQAPGKEREFVTAIN | | | | | | | | | | | |
| | | YVGGRTYADSVRGRFTI | | | | | | | | | | | |
| | | SRDDTKNTVYLQMNSL | | | | | | | | | | | |
| | | KPEDTAVYYCAAGLQY | | | | | | | | | | | |
| | | GITSLRTRNYNYWGQGT | | | | | | | | | | | |
| | | QVTVSSEPKTPKGGCGG | | | | | | | | | | | |
| | | GLEHHHHHH | | | | | | | | | | | |
| G10 | Trypsin | MASMTGGQQMGRNSAQ | SEQ ID | / | / | 2 | No | / | / | / | | | / |
| | | VQLVESGGGLVQAGGSL | NO: 10 | | | | | | | | | | |
| | | RLSCAASGSIFSINSMGW | | | | | | | | | | | |
| | | YRQAPGIERELVAHMPT | | | | | | | | | | | |
| | | GGNTNYLDSVKGRFVIS | | | | | | | | | | | |
| | | RDDDKKTVYLQMNSLT | | | | | | | | | | | |
| | | PEDTAVYYCHAVITTVG | | | | | | | | | | | |
| | | RTGVRTYSYWARGPRSP | | | | | | | | | | | |
| | | SSEPKTPKGGCGGGLEH | | | | | | | | | | | |
| | | HHHHH | | | | | | | | | | | |
| G11 | Trypsin/ | MASMTGGQQMGRNSAQ | SEQ ID | / | 0 | / | No | / | / | / | | | / |
| | Chymo | VQLVESGGGLVQAGGSL | NO: 11 | | | | | | | | | | |
| | | RLSCAASGRTFNSGILG | | | | | | | | | | | |
| | | WFRQAPGKDREFVAAIG | | | | | | | | | | | |
| | | WSAGSTYYSDSVKGRFT | | | | | | | | | | | |
| | | ISRDITKNTVFLQMNSLK | | | | | | | | | | | |
| | | PEDTAVYYCADKKYYY | | | | | | | | | | | |
| | | GREASSNVYEYWGQGT | | | | | | | | | | | |
| | | QVTVSSEPKTPKGGCGG | | | | | | | | | | | |
| | | GLEHHHHHH | | | | | | | | | | | |
| G12 | Chymo | MASMTGGQQMGRNSAQ | SEQ ID | 1 | 0 | 2 | Yes | / | 4.207 | / | | | DSS |
| | | VQLVESGGGLVQAGGSL | NO: 12 | | | | | | | | | | |
| | | RLSCAASRSTFRINAAG | | | | | | | | | | | |
| | | WYRQAPGKERELVARIS | | | | | | | | | | | |
| | | SGGSTNYADSVKGRFTIS | | | | | | | | | | | |
| | | RDNAKNTVYLQMNSLK | | | | | | | | | | | |
| | | PEDTAVYYCNVPYYRED | | | | | | | | | | | |
| | | GYEYDAWGQGTQVTVS | | | | | | | | | | | |
| | | SEPKTPKGGCGGGLEHH | | | | | | | | | | | |
| | | HHHH | | | | | | | | | | | EDC |
| G13 | Chymo | MASMTGGQQMGRNSAD | SEQ ID | / | / | 2.00E+00 | Yes | Yes | 6.735 | 4.74E+05 | 1.70E−04 | 3.60E−10 | DSS |
| | | VQLVESGGGVVQAGGSL | NO: 13 | | | | | | | | | | |
| | | RLSCAASGRTFSDYAMG | | | | | | | | | | | |
| | | WFRQAPGKEREFVAGVS | | | | | | | | | | | |
| | | WSGVDTYYADSVKGRF | | | | | | | | | | | |
| | | TISRDNAKNTLYVQMNS | | | | | | | | | | | |
| | | LKPEDTAVYYCAAQRY | | | | | | | | | | | |
| | | YHGHAKNMRYDYWGQ | | | | | | | | | | | |
| | | GTQVTVSSEPKTPKGGC | | | | | | | | | | | |
| | | GGGLEHHHHHH | | | | | | | | | | | |
| G14 | Chymo | MASMTGGQQMGRNSAE | SEQ ID | 2 | 0 | 2 | Yes | / | 5.274 | / | | | DSS |
| | | VQLVESGGGLVQAGGSL | NO: 14 | | | | | | | | | | |
| | | RLSCAASGSTFDTNPIGW | | | | | | | | | | | |
| | | YRQAPGKQRDLVAMITS | | | | | | | | | | | |
| | | GGHTNYADSVKGRFTIS | | | | | | | | | | | |
| | | RDNAKNTVYLQMNSLK | | | | | | | | | | | |
| | | PEDTAVYYCTVPHYRED | | | | | | | | | | | |
| | | GYEYHFWGQGTQVTVS | | | | | | | | | | | |
| | | SEPKTPKGGCGGGLEHH | | | | | | | | | | | |
| | | HHHH | | | | | | | | | | | |
| | | | | | | | | | | | | | EDC |
| G15 | Chymo | MASMTGGQQMGRNSAQ | SEQ ID | 2 | 0 | 2 | Yes | Yes | 4.606 | / | | | / |
| | | VQLVESGGGLVQAGGSL | NO: 15 | | | | | | | | | | |
| | | RLSCAASGSTFDTNPIGW | | | | | | | | | | | |
| | | YRQAPGKQRDLVAMITS | | | | | | | | | | | |
| | | GGHTNYADSVKGRFTIS | | | | | | | | | | | |
| | | RDNAKNTVYLQMNSLK | | | | | | | | | | | |
| | | PEDTAVYYCTVPHYRED | | | | | | | | | | | |
| | | GYEYHCWGQGTQVTVS | | | | | | | | | | | |
| | | SEPKTPKGGCGGGLEHH | | | | | | | | | | | |
| | | HHHH | | | | | | | | | | | |
| G16 | Cyhmo | MASMTGGQQMGRNSAE | SEQ ID | 2 | 1 | / | Yes | / | 5.684 | / | | | / |
| | | VQLVESGGGLVQPGGSL | NO: 16 | | | | | | | | | | |
| | | RLSCAASGSTFSINAIGW | | | | | | | | | | | |
| | | YRQAPGKEREFVAALR | | | | | | | | | | | |
| | | WPGNIWYYADFVEGRIT | | | | | | | | | | | |
| | | ISRDNAKNTVYLQMNSL | | | | | | | | | | | |
| | | KPEDTAVYYCAARPENR | | | | | | | | | | | |
| | | GSYRDAATYDFWGQGT | | | | | | | | | | | |
| | | QVTVSSEPKTPKGGCGG | | | | | | | | | | | |
| | | GLEHHHHHH | | | | | | | | | | | |
| G17 | Chymo | MASMTGGQQMGRNSAE | SEQ ID | 2 | 2 | 2 | Yes | Yes | 9.81 | 1.34E+06 | 2.92E−05 | 2.17E−11 | / |
| | | VQLVESGGGLVQAGGSL | NO: 17 | | | | | | | | | | |
| | | RLSCAASGVTISYWVMG | | | | | | | | | | | |
| | | WFRQAPGKEREFVARIS | | | | | | | | | | | |
| | | WGGERTYYADSVKGRF | | | | | | | | | | | |
| | | AISRDNAKNTVYLQMNS | | | | | | | | | | | |
| | | LNAEDTAVYYCAADRT | | | | | | | | | | | |
| | | GWGHSNSRSEYDYWGQ | | | | | | | | | | | |
| | | GTQVTVSSEPKTPKGGC | | | | | | | | | | | |
| | | GGGLEHHHHHH | | | | | | | | | | | |
| G18 | Trypsin/ | MASMTGGQQMGRNSAQ | SEQ ID | / | 0 | / | Yes | Yes | / | / | | | / |
| | Chymo | VQLVESGGGLVQAGASL | NO: 18 | | | | | | | | | | |
| | | RLTCGPSGRSVGLYTMG | | | | | | | | | | | |
| | | WFRQAPGKEREFVAGVT | | | | | | | | | | | |
| | | YLGDTTTYSDAVKGRFT | | | | | | | | | | | |
| | | ISRENNKNTVYLRMNSL | | | | | | | | | | | |
| | | KPEDTAVYYCTATATG | | | | | | | | | | | |
| | | WGSPIPSAPGRWDYWG | | | | | | | | | | | |
| | | QGTQVTVSSEPKTPKGG | | | | | | | | | | | |
| | | CGGGLEHHHHHH | | | | | | | | | | | |
| G19 | Trypsin/ | MASMTGGQQMGRNSAQ | SEQ ID | 1 | 0 | 1 | Yes | / | 9 | / | | | / |
| | Chymo | VQLVESGGGLVQAGGSL | NO: 19 | | | | | | | | | | |
| | | RLSCAASGSTFSTNAVD | | | | | | | | | | | |
| | | WYRQAPGNQRDLVATIT | | | | | | | | | | | |
| | | SGGHTNYADSVKGRFTI | | | | | | | | | | | |
| | | SRDNAKNTVYLQMNSL | | | | | | | | | | | |
| | | KPEDTAVYYCAVPHYRE | | | | | | | | | | | |
| | | DGYEYRFWGQGTQVTV | | | | | | | | | | | |
| | | SSEPKTPKGGCGGGLEH | | | | | | | | | | | |
| | | HHHHH | | | | | | | | | | | |
| G20 | Trypsin | MASMTGGQQMGRNSAD | SEQ ID | 1 | / | / | Yes | / | 2.434 | / | | | / |
| | | VQLVESGGGLVQAGGSL | NO: 20 | | | | | | | | | | |
| | | RLSCAASERTFSRYMLG | | | | | | | | | | | |
| | | WFRQAPGKEREFVGVM | | | | | | | | | | | |
| | | GWSDSDTYYGDAVKGR | | | | | | | | | | | |
| | | FTISRDNVKNTIYLQMKS | | | | | | | | | | | |
| | | LKPEDTAVYYCAASAYG | | | | | | | | | | | |
| | | STRNHKLYEYWGQGTQ | | | | | | | | | | | |
| | | VTVSSEPKTPKGGCGGG | | | | | | | | | | | |
| | | LEHHHHHH | | | | | | | | | | | |
| G21 | Trypsin/ | MASMTGGQQMGRNSAD | SEQ ID | / | 0 | 2 | Yes | Yes | / | / | | | / |
| | Chymo | VQLVESGGGSVQAGGSL | NO: 21 | | | | | | | | | | |
| | | RLSCAASGRTFSNYAMA | | | | | | | | | | | |
| | | WFRQAPGKEREFVAAVS | | | | | | | | | | | |
| | | RSGTNLYYADSVKGRFT | | | | | | | | | | | |
| | | ISRDTAENTMYLQMNSL | | | | | | | | | | | |
| | | KPEDTAVYYCAAGLAER | | | | | | | | | | | |
| | | WGIGVQPRSEFLTTGAR | | | | | | | | | | | |
| | | GPRSPSSEPKTPKGGCGG | | | | | | | | | | | |
| | | GLEHHHHHH | | | | | | | | | | | |
| G22 | Trypsin | MASMTGGQQMGRNSAQ | SEQ ID | 1 | / | 1 | Yes | / | 2.071 | 3.09E+04 | 1.25E−03 | 4.05E−08 | / |
| | | VQLVESGGGSVQAGGSL | NO: 22 | | | | | | | | | | |
| | | RLSCAASGRTFSSYSMA | | | | | | | | | | | |
| | | WFRQAPGKEREFVAVM | | | | | | | | | | | |
| | | NCRYGDTDYPDSVKGRF | | | | | | | | | | | |
| | | TMSRDNAKNTLYLQMN | | | | | | | | | | | |
| | | SLKPEDTAVYYCAAKLI | | | | | | | | | | | |
| | | AYCGSGYYYRRNDYGY | | | | | | | | | | | |
| | | WGQGTQVTVSSEPKTPK | | | | | | | | | | | |
| | | GGCGGGLEHHHHHH | | | | | | | | | | | |
| G23 | Trypsin | MASMTGGQQMGRNSAQ | SEQ ID | 1 | / | / | Yes | / | 2.543 | / | | | / |
| | | VQLVESGGGLVQPGGSL | NO: 23 | | | | | | | | | | |
| | | RLSCAASGFTFSVNTMS | | | | | | | | | | | |
| | | WVRQAPGKGREWVSGI | | | | | | | | | | | |
| | | ESHGNTYYSDSVKGRFTI | | | | | | | | | | | |
| | | SRDNAKNTLYLQMNSL | | | | | | | | | | | |
| | | KPEDTAVYYCATGIYGT | | | | | | | | | | | |
| | | TRNWGQGTQVTVS SEPK | | | | | | | | | | | |
| | | TPKGGCGGGLEHHHHH | | | | | | | | | | | |
| | | H | | | | | | | | | | | |
| G24 | Chymo | MASMTGGQQMGRNSAQ | SEQ ID | 1 | 2 | 2 | Yes | Yes | / | / | | | / |
| | | VQLVESGGGLVQAGGTF | NO: 24 | | | | | | | | | | |
| | | GSYVMGWFRQPPGKER | | | | | | | | | | | |
| | | EFVSGIMWNGTSTSTNY | | | | | | | | | | | |
| | | ADSVKGRFTISRDNAKN | | | | | | | | | | | |
| | | TVFLQMNSLQPEDTAVY | | | | | | | | | | | |
| | | YCAASRSSALRTPVPLVE | | | | | | | | | | | |
| | | YWGQGTQVTVSSEPKTP | | | | | | | | | | | |
| | | KGGCGGGLEHHHHHH | | | | | | | | | | | |
| G25 | Trypsin/ | MASMTGGQQMGRNSAE | SEQ ID | 0 | 0 | 1 | Yes | / | 1.575 | / | | | / |
| | Chymo | VQLVESGGGLVQAGGSL | NO: 25 | | | | | | | | | | |
| | | RLSCAASGRTFSGRTFSD | | | | | | | | | | | |
| | | YPMAWFRQAPGKEREFL | | | | | | | | | | | |
| | | ATISTSGSRTYYADSVKG | | | | | | | | | | | |
| | | RFTISRDNAKDTVYLQM | | | | | | | | | | | |
| | | NSLKPEDAAIYYCAARQ | | | | | | | | | | | |
| | | GSYYSDYNRALPGEYDY | | | | | | | | | | | |
| | | WGQGTQVTVSSEPKTPK | | | | | | | | | | | |
| | | GGCGGGLEHHHHHH | | | | | | | | | | | |
| G26 | Chymo | MASMTGGQQMGRNSAQ | SEQ ID | / | / | 0 | Yes | Yes | / | / | | | / |
| | | VQLVESGGGLAQPGGSL | NO: 26 | | | | | | | | | | |
| | | RLSCAASGFTLDAYAIA | | | | | | | | | | | |
| | | WYRQAPGKDREEVACIS | | | | | | | | | | | |
| | | SSGDSTNYAESVKGRFTI | | | | | | | | | | | |
| | | SRDNAKKMGYLQMNSL | | | | | | | | | | | |
| | | KAEDTAIYYCAIDSRGC | | | | | | | | | | | |
| | | AWGGFAYYTFSHWGQG | | | | | | | | | | | |
| | | TQVTVSSEPKTPKGGCG | | | | | | | | | | | |
| | | GGLEHHHHHH | | | | | | | | | | | |
| G27 | Trypsin | MASMTGGQQMGRNSAD | SEQ ID | / | 1 | 0 | Yes | / | 1.365 | / | | | / |
| | | VQLVESGGDLVQAGGSL | NO: 27 | | | | | | | | | | |
| | | RLSCSASGNIFKINDMD | | | | | | | | | | | |
| | | WYRQAPGKQRELVARIS | | | | | | | | | | | |
| | | SSGSTNYADSVKGRFTIS | | | | | | | | | | | |
| | | RDNGKNTVYLQMNRVK | | | | | | | | | | | |
| | | PEDTAVYYCNADVQVS | | | | | | | | | | | |
| | | RNYEYEYWGQGTQVTV | | | | | | | | | | | |
| | | SSEPKTPKGGCGGGLEH | | | | | | | | | | | |
| | | HHHHH | | | | | | | | | | | |
| G28 | Chymo | MASMTGGQQMGRNSAQ | SEQ ID | / | 0 | 0 | Yes | Yes | / | / | | | / |
| | | VQLVESGGGLVQPRGSL | NO: 28 | | | | | | | | | | |
| | | RLSCAASGFTWGDYAIG | | | | | | | | | | | |
| | | WFRQAPGKEREGVSCLS | | | | | | | | | | | |
| | | SSDGSTYYPDSVKDRFTI | | | | | | | | | | | |
| | | STDNAKNTVYLQMTNL | | | | | | | | | | | |
| | | KPDDTAIYYCAAREGPG | | | | | | | | | | | |
| | | ASWYCSVNGYLTQPDS | | | | | | | | | | | |
| | | WGQGTQVTVSSEPKTPK | | | | | | | | | | | |
| | | GGCGGGLEHHHHHH | | | | | | | | | | | |
| G29 | Trypsin | MASMTGGQQMGRNSAQ | SEQ ID | 1 | 0 | 2 | Yes | / | 0, No | / | | | / |
| | | VQLVESGGGLVQAGDSL | NO: 29 | | | | | | binding | | | | |
| | | LLSCGTSGRTFSSNTMG | | | | | | | | | | | |
| | | WFRQAPGKGREFVATIT | | | | | | | | | | | |
| | | ASGRGTNYGDSVRGRFT | | | | | | | | | | | |
| | | ISRDNDKNTVYLQMNNL | | | | | | | | | | | |
| | | KPDDTGVYTCAASDSPY | | | | | | | | | | | |
| | | GSRWIEAYGYWGQGTQ | | | | | | | | | | | |
| | | VTVSSEPKTPKGGCGGG | | | | | | | | | | | |
| | | LEHHHHHH | SEQ ID | / | / | 1 | Yes | / | 4.86 | / | | | / |
| G30 | Chymo | MASMTGGQQMGRNSAQ | NO: 30 | | | | | | | | | | |
| | | VQLVESGGGLVQAGGSL | | | | | | | | | | | |
| | | RLSCAASGRTINNYDMG | | | | | | | | | | | |
| | | WFRQAPGKEREFVAAIT | | | | | | | | | | | |
| | | WSGRDTNYADSVKGRF | | | | | | | | | | | |
| | | TVSRDDAKNTVYLQMN | | | | | | | | | | | |
| | | TLSPEDTAVYYCASARIQ | | | | | | | | | | | |
| | | FYRLVAATRTDYSYWG | | | | | | | | | | | |
| | | QGTQVTVSSEPKTPKGG | | | | | | | | | | | |
| | | CGGGLEHHHHHH | | | | | | | | | | | |
| G31 | Trypsin/ | MASMTGGQQMGRNSAH | SEQ ID | 0 | 0 | 0 | Yes | Yes | / | / | | | / |
| | Chymo | VQLVESGGGLVQAGGSL | NO: 31 | | | | | | | | | | |
| | | RLSCKASESIFKFDAMA | | | | | | | | | | | |
| | | WFRQAPGKERELVACID | | | | | | | | | | | |
| | | NKQRTTYGDSVKGRFTI | | | | | | | | | | | |
| | | SGLDVKNTAYLEMNSLK | | | | | | | | | | | |
| | | PEDTAVYYCTADRSTCF | | | | | | | | | | | |
| | | SNYRLYDYWGQGTQVT | | | | | | | | | | | |
| | | VSSEPKTPKGGCGGGLE | | | | | | | | | | | |
| | | HHHHHH | | | | | | | | | | | |
| G32 | Chymo | MASMTGGQQMGRNSAQ | SEQ ID | / | / | 0 | Yes | / | 0, No | / | | | / |
| | | VQLVESGGGLVRAGDSL | NO: 32 | | | | | | binding | | | | |
| | | RLSCVVSGRPISSYAMA | | | | | | | | | | | |
| | | WFRQAPGKDREVVAGIS | | | | | | | | | | | |
| | | ANGDRTHYADSIKGRFT | | | | | | | | | | | |
| | | VSRDNAKNSMTLQMNK | | | | | | | | | | | |
| | | LKPEDTAVYYCAADSLT | | | | | | | | | | | |
| | | EGGYGLTGDFDYWGQG | | | | | | | | | | | |
| | | TQVTVSSEPKTPKGGCG | | | | | | | | | | | |
| | | GGLEHHHHHH | | | | | | | | | | | |
| G33 | Chymo | MASMTGGQQMGRNSAE | SEQ ID | 0 | 1 | 0 | Yes | / | 0, No | / | | | / |
| | | VQLVESGGSLRLSCSVS | NO: 33 | | | | | | binding | | | | |
| | | GGPFTSNGMGWYRQAP | | | | | | | | | | | |
| | | GKEREWVAAITNSGSAN | | | | | | | | | | | |
| | | YADSVKGRFTVSMVNA | | | | | | | | | | | |
| | | NNTMYLQMNNLKPDDT | | | | | | | | | | | |
| | | AVYYCNVAGWPHGYW | | | | | | | | | | | |
| | | GQGTQVTVSSEPKTPKG | | | | | | | | | | | |
| | | GCGGGLEHHHHHH | | | | | | | | | | | |
| G34 | Trypsin/ | MASMTGGQQMGRNSAQ | SEQ ID | / | / | 1 | Yes | Yes | 5.316 | 2.63E+04 | 4.62E−04 | 1.76E−08 | / |
| | Chymo | VQLVESGGGLVQAGDSL | NO: 34 | | | | | | | | | | |
| | | RLSCAASGRTFSRYAMA | | | | | | | | | | | |
| | | WFRQAPGKEREFVAGIS | | | | | | | | | | | |
| | | WTGRFTYYADSVKGRFT | | | | | | | | | | | |
| | | ISRDDAKNTVYLQMNNL | | | | | | | | | | | |
| | | KPEDTGLYFCKVGDPYG | | | | | | | | | | | |
| | | VGLREYEWWGPGTQVT | | | | | | | | | | | |
| | | VSSEPKTPKGGCGGGLE | | | | | | | | | | | |
| | | HHHHHH | | | | | | | | | | | |
| G35 | Chymo | MASMTGGQQMGRNSAQ | SEQ ID | / | 1 | / | Yes | Yes | / | / | | | / |
| | | VQLVESGGGLVQAGGSL | NO: 35 | | | | | | | | | | |
| | | RLSCAASGRTSRSFAMG | | | | | | | | | | | |
| | | WFRQAPGKGRDFVAAM | | | | | | | | | | | |
| | | TEFGTTYYADSVKGRFTI | | | | | | | | | | | |
| | | SRDNAKNTVYLQMNVL | | | | | | | | | | | |
| | | QSEDTAVYYCAAHWDN | | | | | | | | | | | |
| | | TQWYVYEVGGYEHWG | | | | | | | | | | | |
| | | QGTQVTVSSEPKTPKGG | | | | | | | | | | | |
| | | CGGGLEHHHHHH | | | | | | | | | | | |
| G36 | Trypsin/ | MASMTGGQQMGRNSAQ | SEQ ID | 2 | 0 | 0 | Yes | / | 0, No | / | | | / |
| | Chymo | VQLVESGGSLRLSCAAS | NO: 36 | | | | | | binding | | | | |
| | | GFALSNSYMKWVRQAP | | | | | | | | | | | |
| | | GKGPEWVSTIYADGSTY | | | | | | | | | | | |
| | | YTDSVKGRFITSRDNSK | | | | | | | | | | | |
| | | NTMYLQMSDLKPEDTA | | | | | | | | | | | |
| | | VYYCANPSAKGQGTQV | | | | | | | | | | | |
| | | TVSSEPKTPKGGCGGGL | | | | | | | | | | | |
| | | EHHHHHH | | | | | | | | | | | |
| G37 | Chymo | MASMTGGQQMGRNSAE | SEQ ID | / | 0 | / | Yes | / | 1.785 | / | | | / |
| | | VQLVESGGGLVQAGDSL | NO: 37 | | | | | | | | | | |
| | | RLSCVASGDTFTSYTVG | | | | | | | | | | | |
| | | WFRQAPGKEQEFVAGIS | | | | | | | | | | | |
| | | WSGRSTDYADFVKGRA | | | | | | | | | | | |
| | | TISKDIAKVSLQMNALKP | | | | | | | | | | | |
| | | EDTAVYSCAAKKVDWS | | | | | | | | | | | |
| | | SDYVTNYDYDYRGRGT | | | | | | | | | | | |
| | | QVTVSSEPKTPKGGCGG | | | | | | | | | | | |
| | | GLEHHHHHH | | | | | | | | | | | |
| G38 | Chymo | MASMTGGQQMGRNSAE | SEQ ID | / | 0 | / | Yes | / | 0, No | / | | | / |
| | | VQLVESGGGLVQAGGSL | NO: 38 | | | | | | binding | | | | |
| | | RLSCVASGHTDCISGMG | | | | | | | | | | | |
| | | WYRQAPGKERELVAVLI | | | | | | | | | | | |
| | | GGGNTYYGDSVKGRFTI | | | | | | | | | | | |
| | | SKDKAKNTLYLQMKTL | | | | | | | | | | | |
| | | KPEDMAVYYCTADDHG | | | | | | | | | | | |
| | | SECPNKEMSSTATYWGQ | | | | | | | | | | | |
| | | GTQVTVSSEPKTPKGGC | | | | | | | | | | | |
| | | GGGLEHHHHHH | | | | | | | | | | | |
| G39 | Trypsin/ | MASMTGGQQMGRNSAE | SEQ ID | 1 | / | 2 | Yes | / | 1.653 | / | | | / |
| | Chymo | VQLVESGGELVQSGSSL | NO: 39 | | | | | | | | | | |
| | | RLSCAASGFDLDDYAIG | | | | | | | | | | | |
| | | WFRQAPGKEREGVSCTS | | | | | | | | | | | |
| | | TSDGPTSYLDSVKGRFTF | | | | | | | | | | | |
| | | SRDNAKNTLYLQMNSL | | | | | | | | | | | |
| | | KPEDTAVYYCAAISHIFA | | | | | | | | | | | |
| | | EDAPAMGLCWDQRSAF | | | | | | | | | | | |
| | | WYWGQGTQVTVSSEPK | | | | | | | | | | | |
| | | TPKGGCGGGLEHHHHH | | | | | | | | | | | |
| | | H | | | | | | | | | | | |
| G40 | Chymo | MASMTGGQQMGRNSAE | SEQ ID | 1 | 0 | 0 | Yes | Yes | / | / | | | / |
| | | VQLVESGGGLVQPGGSL | NO: 40 | | | | | | | | | | |
| | | TLSCAASGFHLDNTAIA | | | | | | | | | | | |
| | | WFRQAPGKEREGVSCLS | | | | | | | | | | | |
| | | SRDGSTFYQYSLKDRFTI | | | | | | | | | | | |
| | | SGDNAKNTVYLQMKGL | | | | | | | | | | | |
| | | KPEDTATYYCAAALGID | | | | | | | | | | | |
| | | SQRTVIAGCPKRYFAAW | | | | | | | | | | | |
| | | GQGTQVTVSSEPKTPKG | | | | | | | | | | | |
| | | GCGGGLEHHHHHH | | | | | | | | | | | |
| G41 | Chymo | MASMTGGQQMGRNSAD | SEQ ID | 1 | 0 | 1 | Yes | / | 1.02 | / | | | / |
| | | VQLVESGGGLVQAGGSL | NO: 41 | | | | | | | | | | |
| | | RLSCVASGHTVSNYAM | | | | | | | | | | | |
| | | AWFRQAPGKEREFVAGI | | | | | | | | | | | |
| | | SWRASITYYRDSVKGRF | | | | | | | | | | | |
| | | TISRDNAKNTVYLQMSS | | | | | | | | | | | |
| | | LKPEDTAVYYCASDKTH | | | | | | | | | | | |
| | | YVSRGTSLVEYDYWGQ | | | | | | | | | | | |
| | | GTQVTVSSEPKTPKGGC | | | | | | | | | | | |
| | | GGGLEHHHHHH | | | | | | | | | | | |
| G42 | Trypsin/ | MASMTGGQQMGRNSAH | SEQ ID | 1 | 0 | 2 | Yes | / | 2.63 | / | / | / | / |
| | Chymo | VQLVESGGGFVQAGGSL | NO: 42 | | | | | | | | | | |
| | | RLSCEASGRTFNVYTMG | | | | | | | | | | | |
| | | WFRQAPGKEREFVGSIS | | | | | | | | | | | |
| | | WNGGSTYYADSVKGRF | | | | | | | | | | | |
| | | TISRDNAKNTVYLQMNS | | | | | | | | | | | |
| | | LEPEDTAVYYCAARRQS | | | | | | | | | | | |
| | | HLRLDLSVIDAWGKGTQ | | | | | | | | | | | |
| | | VTVSSEPKTPKGGCGGG | | | | | | | | | | | |
| | | LEHHHHHH | | | | | | | | | | | |
| G43 | Chymo | MASMTGGQQMGRNSAE | SEQ ID | / | 2 | 2 | Yes | / | 4.32 | / | / | / | / |
| | | VQLVESGGGLVQAGGSL | NO: 43 | | | | | | | | | | |
| | | RLSCATSGRTSSTYAMG | | | | | | | | | | | |
| | | WFRQRPGKEREFVATIH | | | | | | | | | | | |
| | | WGVGSTIYADSVKGRFT | | | | | | | | | | | |
| | | LSRDNAQNTVYLQMNS | | | | | | | | | | | |
| | | LKPEDTAVYYCAASTYR | | | | | | | | | | | |
| | | IGSYDVSTSQGYNYWGQ | | | | | | | | | | | |
| | | GTQVTVSSEPKTPKGGC | | | | | | | | | | | |
| | | GGGLEHHHHHH | | | | | | | | | | | |
| G44 | Chymo | MASMTGGQQMGRNSAD | SEQ ID | / | 2 | / | Yes | / | 3.058 | / | | | DSS |
| | | VQLVESGGGLVQAGGSL | NO: 44 | | | | | | | | | | |
| | | RLSCVASGPIFSFSTGGW | | | | | | | | | | | |
| | | YRQAPGKQRELVAALTG | | | | | | | | | | | |
| | | GGNTNYADSVKGRFTIS | | | | | | | | | | | |
| | | RDNAKNTVYLQMNLLK | | | | | | | | | | | |
| | | PEDTAVYYCQVMYYSG | | | | | | | | | | | |
| | | YDGYESTSWGQGTQVT | | | | | | | | | | | |
| | | VSSEPKTPKGGCGGGLE | | | | | | | | | | | |
| | | HHHHHH | | | | | | | | | | | |
| G45 | Chymo | MASMTGGQQMGRNSAE | SEQ ID | 1 | 1 | 2 | Yes | / | 3.171 | / | | | / |
| | | VQLVESGGGLVQPGGSL | NO: 45 | | | | | | | | | | |
| | | RLSCAASGSIFSINSMGW | | | | | | | | | | | |
| | | YRQAPGKQRELVAAITS | | | | | | | | | | | |
| | | GGSTNYANSVKGRFTIS | | | | | | | | | | | |
| | | RNNARNTVWLQMNSLK | | | | | | | | | | | |
| | | PEDTAVYYCNADLNVV | | | | | | | | | | | |
| | | RGYSGDYHGSSDYWGQ | | | | | | | | | | | |
| | | GTQVTVSSEPKTPKGGC | | | | | | | | | | | |
| | | GGGLEHHHHHH | | | | | | | | | | | |
| G46 | Chymo | MASMTGGQQMGRNSAD | SEQ ID | / | 2 | / | Yes | / | 0, No | / | | | / |
| | | VQLVESGGGLVQAGGSL | NO: 46 | | | | | | binding | | | | |
| | | RLSCAASGRTFSRYHMG | | | | | | | | | | | |
| | | WFRQAPGKERDVVAAIS | | | | | | | | | | | |
| | | WSGDSTYYADSVKGRFT | | | | | | | | | | | |
| | | ISKDNAKNTVYLQMDNL | | | | | | | | | | | |
| | | KPEDTAVYYCNVRGGV | | | | | | | | | | | |
| | | LRPYDYWGQGTQVTVS | | | | | | | | | | | |
| | | SEPKTPKGGCGGGLEHH | | | | | | | | | | | |
| | | HHHH | | | | | | | | | | | |
| G47 | Chymo | MASMTGGQQMGRNSAQ | SEQ ID | 1 | 0 | 1 | Yes | Yes | 3.86 | 3.91E+05 | 1.27E−02 | 3.24E−08 | DSS |
| | | VQLVESGGGLVQAGGSL | NO: 47 | | | | | | | | | | |
| | | RLSCAASERIFSNYAMG | | | | | | | | | | | |
| | | WFRQAPGKEREFVASIR | | | | | | | | | | | |
| | | GSGSQTSYADSVKGRFTI | | | | | | | | | | | |
| | | SRDGAKDTVDLQMNSL | | | | | | | | | | | |
| | | KPEDTAVYYCSAKKYC | | | | | | | | | | | |
| | | GSTYNRAEGYDYWGQG | | | | | | | | | | | |
| | | TQVTVSSEPKTPKGGCG | | | | | | | | | | | |
| | | GGLEHHHHHH | | | | | | | | | | | |
| | | | | | | | | | | | | | EDC |
| G48 | Chymo | MASMTGGQQMGRNSAQ | SEQ ID | / | 0 | 0 | Yes | Yes | 1.601 | 5.93E+03 | 7.53E−04 | 1.26E−07 | / |
| | | VQLVESGGGLVQAGGSL | NO: 48 | | | | | | | | | | |
| | | RLSCAASGRTFSTLSMG | | | | | | | | | | | |
| | | WFRQAPGQGREFVGGIN | | | | | | | | | | | |
| | | YDGSSVEYADSVKGRFT | | | | | | | | | | | |
| | | ISRDNAKNMMYLQMNS | | | | | | | | | | | |
| | | LKPEDTAAYYCASSRGY | | | | | | | | | | | |
| | | NTGTNPLGYDVWGQGT | | | | | | | | | | | |
| | | QVTVSSEPKTPKGGCGG | | | | | | | | | | | |
| | | GLEHHHHHH | | | | | | | | | | | |
| G49 | Chymo | MASMTGGQQMGRNSAE | SEQ ID | 2 | 0 | / | Yes | / | 5.545 | / | | | DSS |
| | | VQLVESGGGLVQAGGSL | NO: 49 | | | | | | | | | | |
| | | RLSCAASRSTFSINAAG | | | | | | | | | | | |
| | | WYRQAPGKQRELVAAIS | | | | | | | | | | | |
| | | SGGSANYADSVKGRFIIS | | | | | | | | | | | |
| | | RDNAKNTVYLQMNSLK | | | | | | | | | | | |
| | | PEDTAVYYCRVPYYRDD | | | | | | | | | | | |
| | | GYEYYSWGQGTQVTVS | | | | | | | | | | | EDC |
| | | SEPKTPKGGCGGGLEHH | | | | | | | | | | | |
| | | HHHH | | | | | | | | | | | |
| G50 | Chymo | MASMTGGQQMGRNSAE | SEQ ID | / | 1 | / | Yes | / | 1.091 | / | | | / |
| | | VQLVESGGGLVQAGGSL | NO: 50 | | | | | | | | | | |
| | | RLSCAASGRTFSRYHMG | | | | | | | | | | | |
| | | WFRQAPGKERDVVAAIS | | | | | | | | | | | |
| | | WSGDSTYYADSVKGRFT | | | | | | | | | | | |
| | | ISKDNAKNTVYLQMDSL | | | | | | | | | | | |
| | | KPEDTAVYYCATLSGW | | | | | | | | | | | |
| | | DGDTIFPAGSWGQGTQV | | | | | | | | | | | |
| | | TVSSEPKTPKGGCGGGL | | | | | | | | | | | |
| | | EHHHHHH | | | | | | | | | | | |
| G51 | Chymo | MASMTGGQQMGRNSAQ | SEQ ID | / | 0 | 2 | Yes | / | 4.449 | / | | | / |
| | | VQLVESGGGLVQAGGSL | NO: 51 | | | | | | | | | | |
| | | KLSCAASGITFSINTIGW | | | | | | | | | | | |
| | | YRQAPGKQREFVAHITS | | | | | | | | | | | |
| | | DSTTYYADSVKARFTISR | | | | | | | | | | | |
| | | DSAKNTVHLQMNNLKP | | | | | | | | | | | |
| | | EDTAVYYCNVNPTWPY | | | | | | | | | | | |
| | | GGEVDYWGQGTQVTVS | | | | | | | | | | | |
| | | SEPKTPKGGCGGGLEHH | | | | | | | | | | | |
| | | HHHH | | | | | | | | | | | |
| G52 | Chymo | MASMTGGQQMGRNSAE | SEQ ID | / | 0 | / | Yes | / | 1 | / | | | / |
| | | VQLVESGGGLVQAGGSL | NO: 52 | | | | | | | | | | |
| | | RLSCAASGSTFSSKPIGW | | | | | | | | | | | |
| | | YRQAPGKGRDLVAAIGG | | | | | | | | | | | |
| | | GSSTFYVDSVKGRFTMS | | | | | | | | | | | |
| | | RDNAKNTVALQMNSLK | | | | | | | | | | | |
| | | PEDTAVYYCNEYLGPKV | | | | | | | | | | | |
| | | LPIGSWGQGTQVTVSSEP | | | | | | | | | | | |
| | | KTPKGGCGGGLEHHHH | | | | | | | | | | | |
| | | HH | | | | | | | | | | | |
| G53 | Chymo | MASMTGGQQMGRNSAE | SEQ ID | 2 | 1 | / | Yes | / | 5.365 | / | | | DSS |
| | | VQLVESGGGLVQAGGSL | NO: [symbol] | | | | | | | | | | |
| | | RLSCVASGFTYSTYTMG | | | | | | | | | | | |
| | | WFRQAPGKEREIVAAKN | | | | | | | | | | | |
| | | WSGARIYYTESVKGRFTI | | | | | | | | | | | |
| | | SRDSGSNTMYLQMDSLK | | | | | | | | | | | |
| | | PEDTAVYYCAARLTWT | | | | | | | | | | | |
| | | DTTTPTTYPYWGQGTQV | | | | | | | | | | | |
| | | TVSSEPKTPKGGCGGGL | | | | | | | | | | | |
| | | EHHHHHH | | | | | | | | | | | |
| G54 | Chymo | MASMTGGQQMGRNSAQ | SEQ ID | 0 | 1 | 0 | Yes | / | 1.537 | / | | | / |
| | | VQLVESGGGLVKPGESL | NO: 54 | | | | | | | | | | |
| | | KLSCVASGETLSSYIMG | | | | | | | | | | | |
| | | WFRQAPGKEREFVAAVS | | | | | | | | | | | |
| | | WSGNQQDYADSVKGRF | | | | | | | | | | | |
| | | TISRDNAEKTVDLQMNS | | | | | | | | | | | |
| | | LNPEDTAVYYCAGDQIG | | | | | | | | | | | |
| | | FWSSRTQAHEYEYWGQ | | | | | | | | | | | |
| | | GTQVTVSSEPKTPKGGC | | | | | | | | | | | |
| | | GGGLEHHHHHH | | | | | | | | | | | |
| G55 | Chymo | MASMTGGQQMGRNSAH | SEQ ID | / | 0 | / | No | / | / | / | | | / |
| | | VQLVESGGGLVQAGGSL | NO: 55 | | | | | | | | | | |
| | | RLSCAASEDTFDNYAVA | | | | | | | | | | | |
| | | WFRQARGKEREFVAVIS | | | | | | | | | | | |
| | | WGGGRSTDYTDSVKGR | | | | | | | | | | | |
| | | FSISRDNAKNTVDLQMS | | | | | | | | | | | |
| | | SLKPDDTAVYYCHAQY | | | | | | | | | | | |
| | | YYEDGYEHESWGQGTQ | | | | | | | | | | | |
| | | VTVSSEPKTPKGGCGGG | | | | | | | | | | | |
| | | LEHHHHHH | | | | | | | | | | | |
| G56 | Trypsin/ | MASMTGGQQMGRNSAH | SEQ ID | 1 | / | 0 | No | / | / | / | | | / |
| | Chymo | VQLVESGFAFSSYAMSW | NO: 56 | | | | | | | | | | |
| | | VRQAPTYGREWVAGIYN | | | | | | | | | | | |
| | | DGSHIYYADSVKGRFSIS | | | | | | | | | | | |
| | | RDNVGNTLYLQLNSLQP | | | | | | | | | | | |
| | | NDTALYRCVQEHARGF | | | | | | | | | | | |
| | | GGWGNPNPTDLVYRAW | | | | | | | | | | | |
| | | GRGTQVTVSSEPKTPKG | | | | | | | | | | | |
| | | GCGGGLEHHHHHH | | | | | | | | | | | |
| G57 | Trypsin | MASMTGGQQMGRNSAE | SEQ ID | 0 | 1 | / | Yes | Yes | / | / | | | / |
| | | VQLVESGGGLVQPGGSL | NO: 57 | | | | | | | | | | |
| | | RLSCAASGFTLDYYAIG | | | | | | | | | | | |
| | | WFRQAPGKEREGVSCIS | | | | | | | | | | | |
| | | SSDGSTYYADSVKGRFTI | | | | | | | | | | | |
| | | SRDNAKNTVDLQMDRL | | | | | | | | | | | |
| | | KPEDTAVYYCAADRGSL | | | | | | | | | | | |
| | | YSSGRARAQDYTYWGR | | | | | | | | | | | |
| | | GTQVTVSSEPKTPKGGC | | | | | | | | | | | |
| | | GGGLEHHHHHH | | | | | | | | | | | |
| G58 | Chymo | MASMTGGQQMGRNSAE | SEQ ID | / | / | 0 | Yes | / | 1.537 | / | | | / |
| | | VQLVESGGGLVQAGGSL | NO: 58 | | | | | | | | | | |
| | | RLSCAGSGDTFSRYTLG | | | | | | | | | | | |
| | | WFRQAPGKEREFVAGIS | | | | | | | | | | | |
| | | WSGGSTSYANSVKGRFT | | | | | | | | | | | |
| | | ISRDNAKNTMYLQMNSL | | | | | | | | | | | |
| | | KPEDTAVYTCAAPGLPG | | | | | | | | | | | |
| | | TVVVGASDFYVYWGQG | | | | | | | | | | | |
| | | TQVTVSSEPKTPKGGCG | | | | | | | | | | | |
| | | GGLEHHHHHH | | | | | | | | | | | |
| G59 | Trypsin/ | MASMTGGQQMGRNSAD | SEQ ID | 1 | / | 0 | Yes | Yes | / | | | | / |
| | Chymo | VQLVESGGSLRLSCAAS | NO: 59 | | | | | | | | | | |
| | | GRTINTVGLAWFRQAPG | | | | | | | | | | | |
| | | QQRDFVAGIEIGGALRY | | | | | | | | | | | |
| | | ADSVQGRFTVSRDNAKN | | | | | | | | | | | |
| | | TMYLQMNSLKPEDTAV | | | | | | | | | | | |
| | | YYCGASRGFNIGINPLGY | | | | | | | | | | | |
| | | GGWGQGTQVTVSSEPKT | | | | | | | | | | | |
| | | PKGGCGGGLEHHHHHH | | | | | | | | | | | |
| G60 | Chymo | MASMTGGQQMGRNSAE | SEQ ID | 0 | / | 0 | Yes | Yes | 2.489 | 4.87E+03 | 5.86E−03 | 1.20E−06 | / |
| | | VQLVESGGSLRLSCAAS | NO: 60 | | | | | | | | | | |
| | | GSGFSSSIIAWYRQAPGK | | | | | | | | | | | |
| | | QRELVAAIGGPGSTNYA | | | | | | | | | | | |
| | | DFVEGRFTISRDNAKNT | | | | | | | | | | | |
| | | GYLQMNNLNPEDTAVY | | | | | | | | | | | |
| | | YCNEVTRSGREYWGQG | | | | | | | | | | | |
| | | TQVTVSSEPKTPKGGCG | | | | | | | | | | | |
| | | GGLEHHHHHH | | | | | | | | | | | |
| G61 | Chymo | MASMTGGQQMGRNSAQ | SEQ ID | / | 0 | 0 | No | / | / | / | | | / |
| | | VQLVESGGSLRLSCVAS | NO: 61 | | | | | | | | | | |
| | | GHTDCISGMGWYRQAP | | | | | | | | | | | |
| | | GKERELVAVLIGGGNTY | | | | | | | | | | | |
| | | YGDSVKGRFTISKDKAK | | | | | | | | | | | |
| | | NTLYLQMKTLKPEDTAV | | | | | | | | | | | |
| | | YYCTADDHGSECPNKE | | | | | | | | | | | |
| | | MSSTSTYWGQGTQVTVS | | | | | | | | | | | |
| | | SEPKTPKGGCGGGLEHH | | | | | | | | | | | |
| | | HHHH | | | | | | | | | | | |
| G62 | Trypsin | MASMTGGQQMGRNSAE | SEQ ID | / | / | 0 | No | / | / | / | | | / |
| | | VQLVESGGALVQAGGSL | NO: 62 | | | | | | | | | | |
| | | RLSCLVSGNIYNIKSVG | | | | | | | | | | | |
| | | WYRQAPGKEREDNVKN | | | | | | | | | | | |
| | | TVDLQMNSLKPEDAAV | | | | | | | | | | | |
| | | YYCNARDSSRPRSLPASP | | | | | | | | | | | |
| | | ESLDGRMDVWGKGTQV | | | | | | | | | | | |
| | | TVSSEPKTPKGGCGGGL | | | | | | | | | | | |
| | | EHHHHHH | | | | | | | | | | | |
| G63 | Trypsin | MASMTGGQQMGRNSAQ | SEQ ID | / | 0 | 0 | No | / | / | / | | | / |
| | | VQLVESGGGLVQPGGSL | NO: 63 | | | | | | | | | | |
| | | RLSCKASGFAFSSYAMS | | | | | | | | | | | |
| | | WVRQAPRYGREWVAGI | | | | | | | | | | | |
| | | YNDGSHIYYADSVKGRF | | | | | | | | | | | |
| | | SISRDNVGNTLYLQLNSL | | | | | | | | | | | |
| | | QPNDTALYRCVQEHERG | | | | | | | | | | | |
| | | FGGWGNPNPTDLVYRA | | | | | | | | | | | |
| | | WGRGTQVTVSSEPKTPK | | | | | | | | | | | |
| | | GGCGGGLEHHHHHH | | | | | | | | | | | |
| G64 | Chymo | MASMTGGQQMGRNSAH | SEQ ID | 2 | 0 | 2 | Yes | / | 5.676 | / | | | / |
| | | VQLVESGGGLVQAGGSL | NO: 64 | | | | | | | | | | |
| | | RLSCKVSGTTFSNSAIGW | | | | | | | | | | | |
| | | YRQAPGNRRELVATINY | | | | | | | | | | | |
| | | GGSTNYADSGKGRFTIS | | | | | | | | | | | |
| | | KDNAKNTVYLQMNSLK | | | | | | | | | | | |
| | | PEDTAVYYCKTTEWRED | | | | | | | | | | | |
| | | GYEYDVWGQGTQVTVS | | | | | | | | | | | |
| | | SEPKTPKGGCGGGLEHH | | | | | | | | | | | |
| | | HHHH | | | | | | | | | | | |
| G65 | Chymo | MASMTGGQQMGRNSAQ | SEQ ID | / | / | 2 | Yes | / | 3.971 | / | | | / |
| | | VQLVESGGGLVQAGGSL | NO: 65 | | | | | | | | | | |
| | | RLSCATSGRTFSTYALG | | | | | | | | | | | |
| | | WFRQRPGKEREFVATIH | | | | | | | | | | | |
| | | WSDGRTLYADSVKGRFT | | | | | | | | | | | |
| | | LSRDNAQNTVYLQMNS | | | | | | | | | | | |
| | | LKPEDTAIYYCAASIYRI | | | | | | | | | | | |
| | | GSYDVSTSQGYDYWGQ | | | | | | | | | | | |
| | | GTQVTVSSEPKTPKGGC | | | | | | | | | | | |
| | | GGGLEHHHHHH | | | | | | | | | | | |
| G66 | Chymo | MASMTGGQQMGRNSAQ | SEQ ID | / | 2 | 2 | Yes | / | 4.291 | / | | | DSS |
| | | VQLVESGGGLVQAGGSL | NO: 66 | | | | | | | | | | |
| | | RLSCATSGRTFSTYAMG | | | | | | | | | | | |
| | | WFRQRPGKEREFVATIH | | | | | | | | | | | |
| | | WSDGRTLYTDSVKGRFT | | | | | | | | | | | |
| | | LSRDNAQNTVYLQMNS | | | | | | | | | | | |
| | | LKPEDTAVYYCAAATYR | | | | | | | | | | | |
| | | IGSYDVSTSQGYNYWGQ | | | | | | | | | | | |
| | | GTQVTVSSEPKTPKGGC | | | | | | | | | | | |
| | | GGGLEHHHHHH | | | | | | | | | | | |
| G67 | Trypsin/ | MASMTGGQQMGRNSAH | SEQ ID | 1 | 0 | / | Yes | / | 3.604 | / | | | / |
| | Chymo | VQLVESGGGLVQAGGSL | NO: 67 | | | | | | | | | | |
| | | RLSCVASGHTVSNYAM | | | | | | | | | | | |
| | | AWFRQAPGKEREFVAGI | | | | | | | | | | | |
| | | SWRATLTYYRDSVKGRF | | | | | | | | | | | |
| | | TISRDNAKNTVYLQMSS | | | | | | | | | | | |
| | | LKPEDTAVYFCASDRTP | | | | | | | | | | | |
| | | YVSRGTSLVEYDYWGQ | | | | | | | | | | | |
| | | GTQVTVSSEPKTPKGGC | | | | | | | | | | | |
| | | GGGLEHHHHHH | | | | | | | | | | | |
| G68 | Chymo | MASMTGGQQMGRNSAE | SEQ ID | 2 | / | 1 | Yes | / | 1.075 | / | | | / |
| | | VQLVESGGGLVQAGGSL | NO: 68 | | | | | | | | | | |
| | | RLSCTASGSIFSVNVMD | | | | | | | | | | | |
| | | WYRQAPGKQREFVATIT | | | | | | | | | | | |
| | | GSGATNYADSVKGRFTI | | | | | | | | | | | |
| | | SRGSAKNTVYLQMNSLK | | | | | | | | | | | |
| | | PDDTAVYYCHNADYRE | | | | | | | | | | | |
| | | DGYEYDNWGQGTQVTV | | | | | | | | | | | |
| | | SSEPKTPKGGCGGGLEH | | | | | | | | | | | |
| | | HHHHH | | | | | | | | | | | |
| G69 | | MASMTGGQQMGRNSAQ | SEQ ID | / | / | 2 | Yes | / | 3.687 | / | | | DSS |
| | | VQLVESGGGLVQAGGSL | NO: 69 | | | | | | | | | | |
| | | RLSCVDSGRTFSSNTMG | | | | | | | | | | | |
| | | WFRQAPGKDRDFVAAIN | | | | | | | | | | | |
| | | RSGVITNYADSVKGRFTI | | | | | | | | | | | |
| | | SRDNAKNTVYLQLNSLK | | | | | | | | | | | |
| | | PEDTAVYYCAARAGGW | | | | | | | | | | | |
| | | PSQIPVEYDRWGQGTQV | | | | | | | | | | | |
| | | TVSSEPKTPKGGCGGGL | | | | | | | | | | | |
| | | EHHHHHH | | | | | | | | | | | |
| G70 | Chymo | MASMTGGQQMGRNSAQ | SEQ ID | 1 | 1 | 2 | Yes | / | 1.755 | / | | | / |
| | | VQLVESGGGLVQAGGSL | NO: 70 | | | | | | | | | | |
| | | RLSCAASGATFSINAIGW | | | | | | | | | | | |
| | | YRQAPGKQRELVAVIKS | | | | | | | | | | | |
| | | GNSINYADSVKGRFTISR | | | | | | | | | | | |
| | | DHAKNTVYLQMNNLKP | | | | | | | | | | | |
| | | EDTAVYYCHADQPPETG | | | | | | | | | | | |
| | | WGTWNDLWGQGTQVT | | | | | | | | | | | |
| | | VSSEPKTPKGGCGGGLE | | | | | | | | | | | |
| | | HHHHHH | | | | | | | | | | | |
| G71 | Trypsin/ | MASMTGGQQMGRNSAQ | SEQ ID | 1 | 2 | / | No | / | / | / | | | / |
| | Chymo | VQLVESGGGSVQAGGSL | NO: 71 | | | | | | | | | | |
| | | RLSCAASGRTSVSYAMG | | | | | | | | | | | |
| | | WFRQAPGKEREFVAAVS | | | | | | | | | | | |
| | | RSGTNLYYADSVKGRFT | | | | | | | | | | | |
| | | ISRHTAENTMYLQMNSL | | | | | | | | | | | |
| | | LPEDTALYYCAADEALR | | | | | | | | | | | |
| | | WGIGTQPRSEFFDYWGQ | | | | | | | | | | | |
| | | GTQVTVSSEPKTPKGGC | | | | | | | | | | | |
| | | GGGLEHHHHHH | | | | | | | | | | | |
| G72 | Chymo | MASMTGGQQMGRNSAQ | SEQ ID | / | / | 1 | Yes | / | 0, No | / | | | / |
| | | VQLVESGGGLVQPGGSL | NO: 72 | | | | | | binding | | | | |
| | | RLSCATSGSTFSINGIGW | | | | | | | | | | | |
| | | YRQVPGIEREFVAGVST | | | | | | | | | | | |
| | | DGKANYADSVAGRFTISI | | | | | | | | | | | |
| | | NDGKNTAYLQMNSLKP | | | | | | | | | | | |
| | | EDTAVYYCNVDSTKGY | | | | | | | | | | | |
| | | YWGQGTQVTVSSEPKTP | | | | | | | | | | | |
| | | KGGCGGGLEHHHHHH | | | | | | | | | | | |
| G73 | Chymo | MASMTGGQQMGRNSAE | SEQ ID | / | / | 1 | Yes | / | 0.9549 | / | | | / |
| | | VQLVESGGGLVQAGGSL | NO: 73 | | | | | | | | | | |
| | | RLSCAASGRTFSDDAMA | | | | | | | | | | | |
| | | WFRQAPGKEREFVAAIS | | | | | | | | | | | |
| | | WHPENTFYADSVKGRFT | | | | | | | | | | | |
| | | ISRDKTKNTEYLQMNSL | | | | | | | | | | | |
| | | KPEDTAVYYCAAGPRLE | | | | | | | | | | | |
| | | IGDYAQYKYWGQGTQV | | | | | | | | | | | |
| | | TVSSEPKTPKGGCGGGL | | | | | | | | | | | |
| | | EHHHHHH | | | | | | | | | | | |
| G74 | Chymo | MASMTGGQQMGRNSAQ | SEQ ID | / | / | 1 | Yes | Yes | / | / | | | / |
| | | VQLVESGGGLVQPGGSL | NO: 74 | | | | | | | | | | |
| | | TLSCAASGSTIDDGIGWF | | | | | | | | | | | |
| | | RQASGKEREGVSCIRLSD | | | | | | | | | | | |
| | | GSKYYRDIVKGRFTISRD | | | | | | | | | | | |
| | | NAKNTVYLQMNSLKPE | | | | | | | | | | | |
| | | DTAVYYCANGPCTGPRA | | | | | | | | | | | |
| | | IAEILYGAWGQGTQVTV | | | | | | | | | | | |
| | | SSEPKTPKGGCGGGLEH | | | | | | | | | | | |
| | | HHHHH | | | | | | | | | | | |
| G75 | Chymo | MASMTGGQQMGRNSAH | SEQ ID | / | / | 0 | Yes | Yes | / | / | | | / |
| | | VQLVESGGGLVQAGASL | NO: 75 | | | | | | | | | | |
| | | RLTCGPSGRSVGLYTMG | | | | | | | | | | | |
| | | WFRQAPGKEREFVAGVT | | | | | | | | | | | |
| | | YLGDTTTYSDAVKGRFT | | | | | | | | | | | |
| | | ISRENNKNTVYLRMNSL | | | | | | | | | | | |
| | | KPEDTAVYYCTATATG | | | | | | | | | | | |
| | | WGSPIPSAPGRWGYWG | | | | | | | | | | | |
| | | QGTQVTVSSEPKTPKGG | | | | | | | | | | | |
| | | CGGGLEHHHHHH | | | | | | | | | | | |
| G76 | Trypsin | MASMTGGQQMGRNSAQ | SEQ ID | / | / | 0 | Yes | / | 0, No | / | | | / |
| | | VQLVESGGGLVQPGGSL | NO: 76 | | | | | | binding | | | | |
| | | RLSCVVSGFPFSEYAMS | | | | | | | | | | | |
| | | WVRQTPEKGREWVSGIY | | | | | | | | | | | |
| | | TDGSETLYENSVKGRFTI | | | | | | | | | | | |
| | | SRDNTKNTLYLQMNNL | | | | | | | | | | | |
| | | KPEDTARYYCKLGDPYG | | | | | | | | | | | |
| | | VGLRDYEYLGHGTQVT | | | | | | | | | | | |
| | | VSSEPKTPKGGCGGGLE | | | | | | | | | | | |
| | | HHHHHH | | | | | | | | | | | |
| G77 | Chymo | MASMTGGQQMGRDPAQ | SEQ ID | 2 | 1 | 2 | Yes | / | 6.012 | / | | | / |
| | | VQLVESGGGLVQAGGSL | NO: 77 | | | | | | | | | | |
| | | RLSCTASRSTFRVNPAG | | | | | | | | | | | |
| | | WYRQAPGKERELVARIT | | | | | | | | | | | |
| | | SGGSTNYADSVKGRFTIS | | | | | | | | | | | |
| | | RDNAKNTVYLQMNSLK | | | | | | | | | | | |
| | | PEDTAVYYCNVPYYME | | | | | | | | | | | |
| | | DGYEHDAWGQGTQVTV | | | | | | | | | | | |
| | | SSEPKTPKGGCGGGLEH | | | | | | | | | | | |
| | | HHHHH | | | | | | | | | | | |
| G78 | Chymo | MASMTGGQQMGRDPAD | SEQ ID | 2 | 0 | 2 | Yes | / | 2.082 | / | | | DSS |
| | | VQLVESGGGLVQAGGSL | NO: 78 | | | | | | | | | | |
| | | RLSCTASQSILYINVMG | | | | | | | | | | | |
| | | WYRQAPGKQRELVAEIP | | | | | | | | | | | |
| | | TGGNTDYADSVKGRFTI | | | | | | | | | | | |
| | | SRDNVKNTVSLQMNSLK | | | | | | | | | | | |
| | | PEDTAVYYCNVRGGVLS | | | | | | | | | | | |
| | | PYDYWGQGTQVTVSSEP | | | | | | | | | | | |
| | | KTPKGGCGGGLEHHHH | | | | | | | | | | | |
| | | HH | | | | | | | | | | | |
| G79 | Chymo | MASMTGGQQMGRDPAD | SEQ ID | 2 | 2 | 2 | Yes | / | 2.07 | / | | | / |
| | | VQLVESGGGLVQAGGSL | NO: 79 | | | | | | | | | | |
| | | RLSCATSGRTFSTYAAG | | | | | | | | | | | |
| | | WFRQRPGKEREFVATIH | | | | | | | | | | | |
| | | WNDGRTLYADSVKGRF | | | | | | | | | | | |
| | | TLSRDNAQNTVYLQMN | | | | | | | | | | | |
| | | SLKPEDTAVYYCAAYTY | | | | | | | | | | | |
| | | RIGSYDVSTSQGYDYWG | | | | | | | | | | | |
| | | QGTQVTVSSEPKTPKGG | | | | | | | | | | | |
| | | CGGGLEHHHHHH | | | | | | | | | | | |
| G80 | chymo | MASMTGGQQMGRDPAQ | SEQ ID | 2 | 1 | 2 | Yes | / | 4.428 | / | | | EDC |
| | | VQLVESGGGLVQAGASL | NO: 80 | | | | | | | | | | |
| | | RLSCAASRGTFSSYTMG | | | | | | | | | | | |
| | | WFRQAPGKERLFVASIS | | | | | | | | | | | |
| | | RDGGTTYYADSVKGRFT | | | | | | | | | | | |
| | | ISRDNAENILYLQMNSLK | | | | | | | | | | | |
| | | PEDTAVYYCAAASRHPS | | | | | | | | | | | |
| | | TWEVWGLEYYYWGQG | | | | | | | | | | | |
| | | TQVTVSSEPKTPKGGCG | | | | | | | | | | | |
| | | GGLEHHHHHH | | | | | | | | | | | |
| G81 | chymo | MASMTGGQQMGRDPAE | SEQ ID | 2 | 1 | 2 | Yes | / | 0, No | / | | | / |
| | | VQLVESGGELVQAGGSL | NO: 81 | | | | | | binding | | | | |
| | | RLSCAASGRTDSVTRMA | | | | | | | | | | | |
| | | WFRQAPGKEREFVAAIT | | | | | | | | | | | |
| | | WSSGYTYYPDSVKGRFT | | | | | | | | | | | |
| | | ISRDNAKNTMYLQMNSL | | | | | | | | | | | |
| | | KAEDTAVYICAAAVGVI | | | | | | | | | | | |
| | | SEYNSWGQGTQVTVSSE | | | | | | | | | | | |
| | | PKTPKGGCGGGLEHHHH | | | | | | | | | | | |
| | | HH | | | | | | | | | | | |
| G82 | Chymo | MASMTGGQQMGRDPAH | SEQ ID | 2 | 1 | 2 | Yes | / | 7.218 | 1.11E+06 | 5.61E−04 | 5.04E−10 | EDC |
| | | VQLVESGGGLVQAGGSL | NO: 82 | | | | | | | | | | |
| | | RLSCAASGKIFSLSTMG | | | | | | | | | | | |
| | | WYRQAPGKQRELVAAL | | | | | | | | | | | |
| | | TSGGSTNYADSVKGRFTI | | | | | | | | | | | |
| | | SRDNAKYTTYLQMNSL | | | | | | | | | | | |
| | | KPEDTAVYYCNVRYYS | | | | | | | | | | | |
| | | GYDGYESNSWGQGTQV | | | | | | | | | | | |
| | | TVSSEPKTPKGGCGGGL | | | | | | | | | | | |
| | | EHHHHHH | | | | | | | | | | | |
| G83 | Trypsin | MASMTGGQQMGRDPAQ | SEQ ID | 2 | 1 | 2 | Yes | / | 8.716 | 9.87E+05 | 7.51E−04 | 7.61E−10 | EDC |
| | | VQLVESGGGLVQAGGSL | NO: 83 | | | | | | | | | | |
| | | RLSCAASRRTFSIYNMG | | | | | | | | | | | |
| | | WFRQAPGKEREFVATIT | | | | | | | | | | | |
| | | RYGDRTYTADSVKGRFT | | | | | | | | | | | |
| | | ISSDQAKNTVYLQMNSL | | | | | | | | | | | |
| | | NPHDTAVYYCAADSAY | | | | | | | | | | | |
| | | SGPDFKHYDYWGQGTQ | | | | | | | | | | | |
| | | VTVSSEPKTPKGGCGGG | | | | | | | | | | | |
| | | LEHHHHHH | | | | | | | | | | | |
| G84 | Chymo | MASMTGGQQMGRDPAD | SEQ ID | 2 | / | 2 | No | / | / | / | | | / |
| | | VQLVESGGGLVQPGGSL | NO: 84 | | | | | | | | | | |
| | | RLSCAASGSTFSINAIGW | | | | | | | | | | | |
| | | YRQAPGKEREFVAALR | | | | | | | | | | | |
| | | WPGNIWYYADFVEGRIT | | | | | | | | | | | |
| | | ISRDNAKNTVYLQMNSL | | | | | | | | | | | |
| | | KPEDTAVYYCAATVGLD | | | | | | | | | | | |
| | | SPPRNEYDYWGQGTQV | | | | | | | | | | | |
| | | TVSSEPKTPKGGCGGGL | | | | | | | | | | | |
| | | EHHHHHH | | | | | | | | | | | |
| G85 | Trypsin | MASMTGGQQMGRDPAH | SEQ ID | 2 | 0 | / | Yes | / | 0, No | / | | | / |
| | | VQLVESGGGLVQPGGSL | NO: 85 | | | | | | binding | | | | |
| | | RLSCAASGFTFSTYAMG | | | | | | | | | | | |
| | | WVRQAPGKGPEWVATI | | | | | | | | | | | |
| | | YSKGDTTHYANSAKGRF | | | | | | | | | | | |
| | | TISRDNARNTLYLQMNS | | | | | | | | | | | |
| | | LKPEDTAVYYCAKGISD | | | | | | | | | | | |
| | | SYLRVESNYRGQGTQVT | | | | | | | | | | | |
| | | VSSEPKTPKGGCGGGLE | | | | | | | | | | | |
| | | HHHHHH | | | | | | | | | | | |
| G86 | Trypsin | MASMTGGQQMGRDPAE | SEQ ID | 2 | / | / | Yes | / | 5.542 | / | | | / |
| | | VQLVESGGGLVQAGDSL | NO: 86 | | | | | | | | | | |
| | | RLSCAASGRTFSSYTMG | | | | | | | | | | | |
| | | WFRQAPGKEREFVAGIR | | | | | | | | | | | |
| | | WSGGSTYFTNYEDSVKG | | | | | | | | | | | |
| | | RFTISKDNAKNTVFLQM | | | | | | | | | | | |
| | | NSLRPEDTAVYYCAFTG | | | | | | | | | | | |
| | | HYSTYDSPQRYDYWGQ | | | | | | | | | | | |
| | | GTQVTVSSEPKTPKGGC | | | | | | | | | | | |
| | | GGGLEHHHHHH | | | | | | | | | | | |
| G87 | Trypsin | MASMTGGQQMGRDPAE | SEQ ID | 0 | 1 | 1 | Yes | / | 0, No | / | | | / |
| | | VQLVESGGGLVQAGDSL | NO: 87 | | | | | | binding | | | | |
| | | RLSCAASGRTFSSYNLG | | | | | | | | | | | |
| | | WFRQAPGKEREFVAVM | | | | | | | | | | | |
| | | NCRYGDTDYPDSVKGRF | | | | | | | | | | | |
| | | TMSRDNAKNTLYLEMN | | | | | | | | | | | |
| | | NLKPEDTAVYYCAAKVL | | | | | | | | | | | |
| | | AYCGSGYYYRRNDYGY | | | | | | | | | | | |
| | | WGQGTQVTVSSEPKTPK | | | | | | | | | | | |
| | | GGCGGGLEHHHHHH | | | | | | | | | | | |
| G88 | Chymo | MASMTGGQQMGRDPAE | SEQ ID | 0 | 1 | 0 | No | / | / | / | | | / |
| | | VQLVESGGGLVKPGESL | NO: 88 | | | | | | | | | | |
| | | KLSCVASGETLSSYIMG | | | | | | | | | | | |
| | | WFRQAPGKEREFVAAVS | | | | | | | | | | | |
| | | WSGNQQDYADSVKGQF | | | | | | | | | | | |
| | | TISRDNAEKTVDLQMNS | | | | | | | | | | | |
| | | LNPEDTAVYYCAGDQM | | | | | | | | | | | |
| | | GFWSSRTQAHEYEYWG | | | | | | | | | | | |
| | | QGTQVTVSSEPKTPKGG | | | | | | | | | | | |
| | | CGGGLEHHHHHH | | | | | | | | | | | |
| G89 | Chymo | MASMTGGQQMGRDPAE | SEQ ID | 0 | / | 2 | Yes | / | 5.736 | / | | | / |
| | | VQLVESGGGLVQAGGSL | NO: 89 | | | | | | | | | | |
| | | SLSCAASGSINSINAMG | | | | | | | | | | | |
| | | WFRQAPGKQRELVATIT | | | | | | | | | | | |
| | | RGGSTNYADSVKGRFTI | | | | | | | | | | | |
| | | SIDNAKNTVYLQMNSLK | | | | | | | | | | | |
| | | PEDTAVYYCNADRGTD | | | | | | | | | | | |
| | | DGWLYDYWGQGTQVT | | | | | | | | | | | |
| | | VSSEPKTPKGGCGGGLE | | | | | | | | | | | |
| | | HHHHHH | | | | | | | | | | | |
| G90 | Trypsin | MASMTGGQQMGRDPAD | SEQ ID | 1 | 0 | 2 | Yes | / | 5.832 | / | | | EDC |
| | | VQLVESGGGLVQAGGSL | NO: 90 | | | | | | | | | | |
| | | RLSCAASGLTFSNYAMG | | | | | | | | | | | |
| | | WFRQAPGKEREFAAGIT | | | | | | | | | | | |
| | | WNGGASHYADSVKGRF | | | | | | | | | | | |
| | | TISRDNAQNTVYLQMNS | | | | | | | | | | | |
| | | LKPEDTAVYYCAARLGS | | | | | | | | | | | |
| | | VAYPGLRYDYWGQGTQ | | | | | | | | | | | |
| | | VTVSSEPKTPKGGCGGG | | | | | | | | | | | |
| | | LEHHHHHH | | | | | | | | | | | |
| G91 | Trypsin/ | MASMTGGQQMGRDPAE | SEQ ID | 0 | / | / | Yes | / | 1.145 | / | | | / |
| | Chymo | VQLVESGGGLVQAGGSL | NO: 91 | | | | | | | | | | |
| | | RLSCAASGRTFSDYPMA | | | | | | | | | | | |
| | | WFRQALGKEREFLATIST | | | | | | | | | | | |
| | | SGSRTMYADSVKGRFTI | | | | | | | | | | | |
| | | SRDNAKNMMYLQMNSL | | | | | | | | | | | |
| | | KPEDAAVYYCAARQGS | | | | | | | | | | | |
| | | YYSDYNRALPGEYHYW | | | | | | | | | | | |
| | | GQGTQVTVSSEPKTPKG | | | | | | | | | | | |
| | | GCGGGLEHHHHHH | | | | | | | | | | | |
| G92 | Chymo | MASMTGGQQMGRDPAD | SEQ ID | 0 | / | 0 | No | / | / | / | | | / |
| | | VQLVESGGGLVKPGESL | NO: 92 | | | | | | | | | | |
| | | KLSCVASGETLSSYIMG | | | | | | | | | | | |
| | | WFRQAPGQGRKFVGGIN | | | | | | | | | | | |
| | | YSGSSVEYADSVKGRFTI | | | | | | | | | | | |
| | | SRDNAKNTMYLQMNSL | | | | | | | | | | | |
| | | KPEDTAAYYCASSRGYN | | | | | | | | | | | |
| | | TGTNPLGYNYWGQGTQ | | | | | | | | | | | |
| | | VTVSSEPKTPKGGCGGG | | | | | | | | | | | |
| | | LEHHHHHH | | | | | | | | | | | |
| G93 | Chymo | MASMTGGQQMGRDPAQ | SEQ ID | 0 | / | / | Yes | / | 2.16 | / | | | / |
| | | VQLVESGGGLVQPGGSL | NO: 93 | | | | | | | | | | |
| | | RLSCAASGSGFSSSIIGW | | | | | | | | | | | |
| | | HRQAPGKQRELVAAIGG | | | | | | | | | | | |
| | | PGSTNYADSVKGRFTISR | | | | | | | | | | | |
| | | DNAKNTAYLQMNNLKP | | | | | | | | | | | |
| | | EDSAVYYCEATTRSGRE | | | | | | | | | | | |
| | | YWGQGTQVTVSSEPKTP | | | | | | | | | | | |
| | | KGGCGGGLEHHHHHH | | | | | | | | | | | |
| G94 | Chymo | MASMTGGQQMGRDPAH | SEQ ID | 0 | 0 | 0 | No | / | / | / | | | / |
| | | VQLVESGGGLVQPGGSL | NO: 94 | | | | | | | | | | |
| | | RLSCVASGFTFSAYAMS | | | | | | | | | | | |
| | | WVRQVPGKGREWISGIY | | | | | | | | | | | |
| | | NDGSNIYYTDSVKGRFSI | | | | | | | | | | | |
| | | SRDNAKNTLYLQMNNL | | | | | | | | | | | |
| | | KPDDTAVYYCTKEHAR | | | | | | | | | | | |
| | | GFGGRGNPNPSDLVYDA | | | | | | | | | | | |
| | | WGQGTQVTVSSEPKTPK | | | | | | | | | | | |
| | | GGCGGGLEHHHHHH | | | | | | | | | | | |
| G95 | Trypsin/ | MASMTGGQQMGRDPAH | SEQ ID | / | 2 | 1 | Yes | / | 3.76 | 1.39E+04 | 1.07E−03 | 7.70E−08 | / |
| | Chymo | VQLVESGGGLVQAGGSL | NO: 95 | | | | | | | | | | |
| | | RLSCAASGRTFSSYAMA | | | | | | | | | | | |
| | | WFRQAVGKEREFVAAV | | | | | | | | | | | |
| | | SRSGTNLYYADSVKGRF | | | | | | | | | | | |
| | | TISRDTAKNTMYLQMNS | | | | | | | | | | | |
| | | LKPEDTALYYCAAGEAL | | | | | | | | | | | |
| | | RWGIGQQPRSEFFDYWG | | | | | | | | | | | |
| | | QGTQVTVSSEPKTPKGG | | | | | | | | | | | |
| | | CGGGLEHHHHHH | | | | | | | | | | | |
| G96 | Chymo | MASMTGGQQMGRDPAD | SEQ ID | / | 0 | / | Yes | / | 6.769 | / | | | EDC |
| | | VQLVESGGGLVQAGGSL | NO: 96 | | | | | | | | | | |
| | | KLSCAAFGVTFDINTIAW | | | | | | | | | | | |
| | | YRQAPGKQREFVAHITS | | | | | | | | | | | |
| | | GGTTYYADSVKARFTMS | | | | | | | | | | | |
| | | RDSAKNTVYLQMNNLK | | | | | | | | | | | |
| | | PEDTAVYYCNVNPTWP | | | | | | | | | | | |
| | | YSGEVDYWGQGTQVTV | | | | | | | | | | | |
| | | SSEPKTPKGGCGGGLEH | | | | | | | | | | | |
| | | HHHHH | | | | | | | | | | | |
| G97 | Chymo | MASMTGGQQMGRDPAE | SEQ ID | / | 2 | / | Yes | / | 3.298 | / | | | / |
| | | VQLVESGGGLVQAGGSL | NO: 97 | | | | | | | | | | |
| | | RLSCTASRSTFRVNPAG | | | | | | | | | | | |
| | | WYRQAPGKERELVARIT | | | | | | | | | | | |
| | | SGGSTNYADSVKGRFTIS | | | | | | | | | | | |
| | | RDNAKNTVYLQMNSLK | | | | | | | | | | | |
| | | PEDTAVYYCNVPCYME | | | | | | | | | | | |
| | | DGYEHDAWGQGTQVTV | | | | | | | | | | | |
| | | SSEPKTPKGGCGGGLEH | | | | | | | | | | | |
| | | HHHHH | | | | | | | | | | | |
| G98 | Chymo | MASMTGGQQMGRDPAQ | SEQ ID | 2 | 2 | 2 | Yes | / | 4.669 | / | | | / |
| | | VQLVESGGGLVQAGDSL | NO: 98 | | | | | | | | | | |
| | | RLSCATSGRTFSTYAAG | | | | | | | | | | | |
| | | WFRQRPGKEREFVATIH | | | | | | | | | | | |
| | | WNDGRTLYADSVKGRF | | | | | | | | | | | |
| | | TLSRDNAQNTVYLQMN | | | | | | | | | | | |
| | | SLKPEDTAVYYCAASTY | | | | | | | | | | | |
| | | RIGSYDVSTSQGYDYWG | | | | | | | | | | | |
| | | QGTQVTVSSEPKTPKGG | | | | | | | | | | | |
| | | CGGGLEHHHHHH | | | | | | | | | | | |
| | | | | | | | | | | | | | CX |
| | | | | | | | | | Cross- | CX | CX | CX | Model |
| | | | | | | | | | linked | residue | residue | Model | Epi- |
| | | | | | | | | ID | Peptides | on GST | on Nbs | Folder | tope |
| | | | | | | | | G1 | / | / | / | / | / |
| | | | | | | | | G2 | / | / | / | / | / |
| | | | | | | | | G3 | / | / | / | / | / |
| | | | | | | | | G4 | / | / | / | / | / |
| | | | | | | | | G5 | / | / | / | / | / |
| | | | | | | | | G6 | / | / | / | / | / |
| | | | | | | | | G7 | / | / | / | / | / |
| | | | | | | | | G8 | / | / | / | / | / |
| | | | | | | | | G9 | RIEAIPQIDK | GST(191) | G9(101) | Seq_ | E1 |
| | | | | | | | | | YLK (SEQ ID | | | 17023 | |
| | | | | | | | | | NO: 2537)(10)- | | | | |
| | | | | | | | | | NTVYLQMNS | | | | |
| | | | | | | | | | LKPEDTAVY | | | | |
| | | | | | | | | | YCAAGLQYG | | | | |
| | | | | | | | | | ITSLR (SEQ | | | | |
| | | | | | | | | | ID NO: | | | | |
| | | | | | | | | | 2538)(11) | | | | |
| | | | | | | | | G10 | / | / | / | / | / |
| | | | | | | | | G11 | / | / | / | / | / |
| | | | | | | | | G12 | YEEHLYERD | GST(40) | G12(90) | Seq_ | E2 |
| | | | | | | | | | EGDKWR | | | 20204 | |
| | | | | | | | | | (SEQ ID NO: | | | | |
| | | | | | | | | | 2539)(13)- | | | | |
| | | | | | | | | | DNAKNTVYL | | | | |
| | | | | | | | | | QMNSLKPED | | | | |
| | | | | | | | | | TAVYYCNVP | | | | |
| | | | | | | | | | YYR (SEQ ID | | | | |
| | | | | | | | | | NO: 2540)(4) | | | | |
| | | | | | | | | | VDFLSKLPE | GST(125) | G12(123) | | |
| | | | | | | | | | MLK (SEQ ID | | | | |
| | | | | | | | | | NO: 2541)(6)- | | | | |
| | | | | | | | | | EDGYEYDA | | | | |
| | | | | | | | | | WGQGTQVT | | | | |
| | | | | | | | | | VSSEPK (SEQ | | | | |
| | | | | | | | | | ID NO: 2542) | | | | |
| | | | | | | | | | (7) | | | | |
| | | | | | | | | | VDFLSKLPE | GST(125) | G12(121) | | |
| | | | | | | | | | MLK (SEQ ID | | | | |
| | | | | | | | | | NO: 2541)(6)- | | | | |
| | | | | | | | | | NTVYLQMNS | | | | |
| | | | | | | | | | LKPEDTAVY | | | | |
| | | | | | | | | | YCNVPYYRE | | | | |
| | | | | | | | | | DGYEYDAW | | | | |
| | | | | | | | | | GQGTQVTVS | | | | |
| | | | | | | | | | SEPK (SEQ ID | | | | |
| | | | | | | | | | NO: 2543)(31) | | | | |
| | | | | | | | | | SDLEVLFQGP | GST(233) | G12(79) | | |
| | | | | | | | | | LGSPEFPGR | | | | |
| | | | | | | | | | (SEQ ID NO: | | | | |
| | | | | | | | | | 2544)(15)- | | | | |
| | | | | | | | | | ISSGGSTNYA | | | | |
| | | | | | | | | | DSVKGR | | | | |
| | | | | | | | | | (SEQ ID NO: | | | | |
| | | | | | | | | | 2545)(14) | | | | |
| | | | | | | | | G13 | RIEAIPQIDK | GST(191) | G13(20) | Seq_ | E3 |
| | | | | | | | | | YLK (SEQ ID | | | 73309 | |
| | | | | | | | | | NO: 2537) | | | | |
| | | | | | | | | | (10)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | KRIEAIPQID | GST(181) | G13(2) | | |
| | | | | | | | | | K (SEQ ID | | | | |
| | | | | | | | | | NO: 2547)(1)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | LLLEYLEEK | GST(27) | G13(2) | | |
| | | | | | | | | | YEEHLYER | | | | |
| | | | | | | | | | (SEQ ID NO: | | | | |
| | | | | | | | | | 2548)(9)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | SSKYIAWPL | GST(197) | G13(2) | | |
| | | | | | | | | | QGWQATFG | | | | |
| | | | | | | | | | GGDHPPK | | | | |
| | | | | | | | | | (SEQ ID NO: | | | | |
| | | | | | | | | | 2549)(3)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | G14 | DFETLKVDF | GST(119) | G14(58) | Seq_ | E2 |
| | | | | | | | | | LSK (SEQ ID | | | 47378 | |
| | | | | | | | | | NO: 2550)(6)- | | | | |
| | | | | | | | | | QAPGKQR | | | | |
| | | | | | | | | | (SEQ ID NO: | | | | |
| | | | | | | | | | 2551)(5) | | | | |
| | | | | | | | | | IAYSKDFETL | GST(113) | G14(90) | | |
| | | | | | | | | | K (SEQ ID | | | | |
| | | | | | | | | | NO: 2552)(5)- | | | | |
| | | | | | | | | | DNAKNTVYL | | | | |
| | | | | | | | | | QMNSLKPED | | | | |
| | | | | | | | | | TAVYYCTVP | | | | |
| | | | | | | | | | HYR (SEQ ID | | | | |
| | | | | | | | | | NO: 2553)(4) | | | | |
| | | | | | | | | | LSCAASGSTF | GST(113) | G14(45) | | |
| | | | | | | | | | DTNPIGWYR | | | | |
| | | | | | | | | | (SEQ ID NO: | | | | |
| | | | | | | | | | 2554)(11)- | | | | |
| | | | | | | | | | IAYSKDFETL | | | | |
| | | | | | | | | | K (SEQ ID | | | | |
| | | | | | | | | | NO: 2552)(5) | | | | |
| | | | | | | | | G15 | / | / | / | / | / |
| | | | | | | | | G16 | / | / | / | / | / |
| | | | | | | | | G17 | / | / | / | / | / |
| | | | | | | | | G18 | / | / | / | / | / |
| | | | | | | | | G19 | / | / | / | / | / |
| | | | | | | | | G20 | / | / | / | / | / |
| | | | | | | | | G21 | / | / | / | / | / |
| | | | | | | | | G22 | / | / | / | / | / |
| | | | | | | | | G23 | / | / | / | / | / |
| | | | | | | | | G24 | / | / | / | / | / |
| | | | | | | | | G25 | / | / | / | / | / |
| | | | | | | | | G26 | / | / | / | / | / |
| | | | | | | | | G27 | / | / | / | / | / |
| | | | | | | | | G28 | / | / | / | / | / |
| | | | | | | | | G29 | / | / | / | / | / |
| | | | | | | | | G30 | / | / | / | / | / |
| | | | | | | | | G31 | / | / | / | / | / |
| | | | | | | | | G32 | / | / | / | / | / |
| | | | | | | | | G33 | / | / | / | / | / |
| | | | | | | | | G34 | / | / | / | / | / |
| | | | | | | | | G35 | / | / | / | / | / |
| | | | | | | | | G36 | / | / | / | / | / |
| | | | | | | | | G37 | / | / | / | / | / |
| | | | | | | | | G38 | / | / | / | / | / |
| | | | | | | | | G39 | / | / | / | / | / |
| | | | | | | | | G40 | / | / | / | / | / |
| | | | | | | | | G41 | / | / | / | / | / |
| | | | | | | | | G42 | / | / | / | / | / |
| | | | | | | | | G43 | / | / | / | / | / |
| | | | | | | | | G44 | DFETLKVDF | GST(119) | G44(58) | Seq_ | E3 |
| | | | | | | | | | LSK (SEQ ID | | | 41521 | |
| | | | | | | | | | NO: 2550)(6)- | | | | |
| | | | | | | | | | QAPGKQR | | | | |
| | | | | | | | | | (SEQ ID NO: | | | | |
| | | | | | | | | | 2551)(5) | | | | |
| | | | | | | | | | IEAIPQIDKYL | GST(191) | G44(79) | | |
| | | | | | | | | | K (SEQ ID | | | | |
| | | | | | | | | | NO: 2555)(9)- | | | | |
| | | | | | | | | | ELVAALTGG | | | | |
| | | | | | | | | | GNTNYADSV | | | | |
| | | | | | | | | | KGR (SEQ ID | | | | |
| | | | | | | | | | NO: 2556)(19) | | | | |
| | | | | | | | | | YLKSSK(SEQ | GST(194) | G44(79) | | |
| | | | | | | | | | ID NO: 2557) | | | | |
| | | | | | | | | | (3)- | | | | |
| | | | | | | | | | ELVAALTGG | | | | |
| | | | | | | | | | GNTNYADSV | | | | |
| | | | | | | | | | KGR (SEQ ID | | | | |
| | | | | | | | | | NO: 2556)(19) | | | | |
| | | | | | | | | | KRIEAIPQID | GST(181) | G44(79) | | |
| | | | | | | | | | K(SEQ ID NO: | | | | |
| | | | | | | | | | 2547)(1)- | | | | |
| | | | | | | | | | ELVAALTGG | | | | |
| | | | | | | | | | GNTNYADSV | | | | |
| | | | | | | | | | KGR (SEQ ID | | | | |
| | | | | | | | | | NO: 2556)(19) | | | | |
| | | | | | | | | G45 | / | / | / | / | / |
| | | | | | | | | G46 | / | / | / | / | / |
| | | | | | | | | G47 | IAYSKDFETL | GST(113) | G47(102 | Seq_ | E2 |
| | | | | | | | | | K(SEQ ID NO: | | | 54055 | |
| | | | | | | | | | 2552)(5)- | | | | |
| | | | | | | | | | DTVDLQMNS | | | | |
| | | | | | | | | | LKPEDTAVY | | | | |
| | | | | | | | | | YCSAK(SEQ | | | | |
| | | | | | | | | | ID NO: | | | | |
| | | | | | | | | | 2558)(11) | | | | |
| | | | | | | | | | IAYSKDFETL | GST(113) | G47(115) | | |
| | | | | | | | | | K (SEQ ID | | | | |
| | | | | | | | | | NO: 2552)(5)- | | | | |
| | | | | | | | | | KYCGSTYNR | | | | |
| | | | | | | | | | (SEQ ID NO: | | | | |
| | | | | | | | | | 2559)(1) | | | | |
| | | | | | | | | | SDLEVLFQGP | GST(222) | G47(91) | | |
| | | | | | | | | | LGSPEFPGR | | | | |
| | | | | | | | | | (SEQ ID NO: | | | | |
| | | | | | | | | | 2545)(4)- | | | | |
| | | | | | | | | | DGAKDTVDL | | | | |
| | | | | | | | | | QMNSLK | | | | |
| | | | | | | | | | (SEQ ID NO: | | | | |
| | | | | | | | | | 2560)(4) | | | | |
| | | | | | | | | | IKGLVQPTR | GST(11) | G47(128) | | |
| | | | | | | | | | (SEQ ID NO: | | | | |
| | | | | | | | | | 2561)(2)- | | | | |
| | | | | | | | | | AEGYDYWG | | | | |
| | | | | | | | | | QGTQVTVSS | | | | |
| | | | | | | | | | EPK(SEQ ID | | | | |
| | | | | | | | | | NO: 2562)(5) | | | | |
| | | | | | | | | G48 | / | / | / | / | / |
| | | | | | | | | G49 | IEAIPQIDKYL | GST(191) | G49(79) | Seq_ | E3 |
| | | | | | | | | | K (SEQ ID | | | 24699 | |
| | | | | | | | | | NO: 2555)(9)- | | | | |
| | | | | | | | | | ELVAAISSGG | | | | |
| | | | | | | | | | SANYADSVK | | | | |
| | | | | | | | | | GR(SEQ ID | | | | |
| | | | | | | | | | NO: 2563)(19) | | | | |
| | | | | | | | | | LERPHRD | GST(239) | G49(90) | | |
| | | | | | | | | | (SEQ IDNO: | | | | |
| | | | | | | | | | 2564)(2)- | | | | |
| | | | | | | | | | DNAKNTVYL | | | | |
| | | | | | | | | | QMNSLKPED | | | | |
| | | | | | | | | | TAVYYCR | | | | |
| | | | | | | | | | (SEQ ID NO: | | | | |
| | | | | | | | | | 2565)(4) | | | | |
| | | | | | | | | | LERPHRD | GST(244) | G49(90) | | |
| | | | | | | | | | (SEQ ID NO: | | | | |
| | | | | | | | | | 2564)(7)- | | | | |
| | | | | | | | | | DNAKNTVYL | | | | |
| | | | | | | | | | QMNSLKPED | | | | |
| | | | | | | | | | TAVYYCR | | | | |
| | | | | | | | | | (SEQ ID NO: | | | | |
| | | | | | | | | | 2565)(4) | | | | |
| | | | | | | | | | LERPHRD(SEQ | GST(244) | G49(101) | | |
| | | | | | | | | | ID NO: | | | | |
| | | | | | | | | | 2564)(7)- | | | | |
| | | | | | | | | | NTVYLQMNS | | | | |
| | | | | | | | | | LKPEDTAVY | | | | |
| | | | | | | | | | YCR (SEQ ID | | | | |
| | | | | | | | | | NO: 2566)(11) | | | | |
| | | | | | | | | | SDLEVLFQGP | GST(233) | G49(79) | | |
| | | | | | | | | | LGSPEFPGR | | | | |
| | | | | | | | | | (SEQ ID NO: | | | | |
| | | | | | | | | | 2545)(15)- | | | | |
| | | | | | | | | | ELVAAISSGG | | | | |
| | | | | | | | | | SANYADSVK | | | | |
| | | | | | | | | | GR (SEQ ID | | | | |
| | | | | | | | | | NO: 2563)(19) | | | | |
| | | | | | | | | | LERPHRD | GST(239) | G49(101) | | |
| | | | | | | | | | (SEQ ID NO: | | | | |
| | | | | | | | | | 2564)(2)- | | | | |
| | | | | | | | | | NTVYLQMNS | | | | |
| | | | | | | | | | LKPEDTAVY | | | | |
| | | | | | | | | | YCR (SEQ ID | | | | |
| | | | | | | | | | NO: 2566)(11) | | | | |
| | | | | | | | | G50 | / | / | / | / | / |
| | | | | | | | | G51 | / | / | / | / | / |
| | | | | | | | | G52 | / | / | / | / | / |
| | | | | | | | | G53 | LLLEYLEEK | GST(27) | G53(80) | Seq_ | E3 |
| | | | | | | | | | YEEHLYER | | | 55403 | |
| | | | | | | | | | (SEQ ID NO: | | | | |
| | | | | | | | | | 2548)(9)- | | | | |
| | | | | | | | | | IYYTESVKGR | | | | |
| | | | | | | | | | (SEQ ID NO: | | | | |
| | | | | | | | | | 2567)(8) | | | | |
| | | | | | | | | | LLLEYLEEK | GST(27) | G53(66) | | |
| | | | | | | | | | YEEHLYER | | | | |
| | | | | | | | | | (SEQ ID NO: | | | | |
| | | | | | | | | | 2548)(9)- | | | | |
| | | | | | | | | | EREIVAAKN | | | | |
| | | | | | | | | | WSGAR(SEQ | | | | |
| | | | | | | | | | ID NO: | | | | |
| | | | | | | | | | 2568)(8) | | | | |
| | | | | | | | | | IKGLVQPTR | GST(11) | G53(66) | | |
| | | | | | | | | | (SEQ ID NO: | | | | |
| | | | | | | | | | 2561)(2)- | | | | |
| | | | | | | | | | EIVAAKNWS | | | | |
| | | | | | | | | | GAR(SEQ ID | | | | |
| | | | | | | | | | NO: 2568)(6) | | | | |
| | | | | | | | | G54 | / | / | / | / | / |
| | | | | | | | | G55 | / | / | / | / | / |
| | | | | | | | | G56 | / | / | / | / | / |
| | | | | | | | | G57 | / | / | / | / | / |
| | | | | | | | | G58 | / | / | / | / | / |
| | | | | | | | | G59 | / | / | / | / | / |
| | | | | | | | | G60 | / | / | / | / | / |
| | | | | | | | | G61 | / | / | / | / | / |
| | | | | | | | | G62 | / | / | / | / | / |
| | | | | | | | | G63 | / | / | / | / | / |
| | | | | | | | | G64 | / | / | / | / | / |
| | | | | | | | | G65 | / | / | / | / | / |
| | | | | | | | | G66 | IEAIPQIDKYL | GST(191) | G66(58) | Seq_ | E3 |
| | | | | | | | | | K (SEQ ID | | | 58516 | |
| | | | | | | | | | NO: 2555)(9)- | | | | |
| | | | | | | | | | QRPGKER | | | | |
| | | | | | | | | | (SEQ ID NO: | | | | |
| | | | | | | | | | 2575)(5) | | | | |
| | | | | | | | | G67 | / | / | / | / | / |
| | | | | | | | | G68 | / | / | / | / | / |
| | | | | | | | | G69 | QAPGKDRDF | GST(194) | G69(58) | Seq_ | E3 |
| | | | | | | | | | VAAINR(SEQ | | | 20239 | |
| | | | | | | | | | ID NO: | | | | |
| | | | | | | | | | 2569)(5)- | | | | |
| | | | | | | | | | YLKSSK (SEQ | | | | |
| | | | | | | | | | ID NO: | | | | |
| | | | | | | | | | 2557)(3) | | | | |
| | | | | | | | | | QAPGKDRDF | GST(191) | G69(58) | | |
| | | | | | | | | | VAAINR (SEQ | | | | |
| | | | | | | | | | ID NO: | | | | |
| | | | | | | | | | 2569)(5)- | | | | |
| | | | | | | | | | RIEAIPQIDK | | | | |
| | | | | | | | | | YLK (SEQ ID | | | | |
| | | | | | | | | | NO: 2537)(10) | | | | |
| | | | | | | | | | NTVYLQLNS | GST(113) | G69(102) | | |
| | | | | | | | | | LKPEDTAVY | | | | |
| | | | | | | | | | YCAAR (SEQ | | | | |
| | | | | | | | | | ID NO: | | | | |
| | | | | | | | | | 2570)(11)- | | | | |
| | | | | | | | | | IAYSKDFETL | | | | |
| | | | | | | | | | K (SEQ ID | | | | |
| | | | | | | | | | NO: 2552)(5) | | | | |
| | | | | | | | | | SGVITNYADS | GST(18 [symbol] | G69 | | |
| | | | | | | | | | VKGR (SEQ | | | | |
| | | | | | | | | | ID NO: | | | | |
| | | | | | | | | | 2571)(12)- | | | | |
| | | | | | | | | | KRIEAIPQID | | | | |
| | | | | | | | | | K (SEQ ID | | | | |
| | | | | | | | | | NO: 2547)(1) | | | | |
| | | | | | | | | G70 | / | / | / | / | / |
| | | | | | | | | G71 | / | / | / | / | / |
| | | | | | | | | G72 | / | / | / | / | / |
| | | | | | | | | G73 | / | / | / | / | / |
| | | | | | | | | G74 | / | / | / | / | / |
| | | | | | | | | G75 | / | / | / | / | / |
| | | | | | | | | G76 | / | / | / | / | / |
| | | | | | | | | G77 | / | / | / | / | / |
| | | | | | | | | G78 | NTVSLQMNS | GST(113) | G78(101) | Seq_ | E2 |
| | | | | | | | | | LKPEDTAVY | | | 6584 | |
| | | | | | | | | | YCNVR (SEQ | | | | |
| | | | | | | | | | ID NO: | | | | |
| | | | | | | | | | 2572)(11)- | | | | |
| | | | | | | | | | IAYSKDFETL | | | | |
| | | | | | | | | | K (SEQ ID | | | | |
| | | | | | | | | | NO: 2552)(5) | | | | |
| | | | | | | | | G79 | / | / | / | / | / |
| | | | | | | | | G80 | DEGDKWR | GST(37) | G80(58) | Seq_ | E3 |
| | | | | | | | | | (SEQ ID NO: | | | 51356 | |
| | | | | | | | | | 2573)(2)- | | | | |
| | | | | | | | | | QAPGKER | | | | |
| | | | | | | | | | (SEQ ID NO: | | | | |
| | | | | | | | | | 2574)(5) | | | | |
| | | | | | | | | | DGGTTYYAD | GST(37) | G80(80) | | |
| | | | | | | | | | SVKGR (SEQ | | | | |
| | | | | | | | | | ID NO: | | | | |
| | | | | | | | | | 2576)(12)- | | | | |
| | | | | | | | | | DEGDKWR | | | | |
| | | | | | | | | | (SEQ ID NO: | | | | |
| | | | | | | | | | 2573)(2) | | | | |
| | | | | | | | | | DPAQVQLVE | GST(181) | G80(13) | | |
| | | | | | | | | | SGGGLVQAG | | | | |
| | | | | | | | | | ASLR (SEQ ID | | | | |
| | | | | | | | | | NO: 2577)(1)- | | | | |
| | | | | | | | | | KRIEAIPQID | | | | |
| | | | | | | | | | K (SEQ ID | | | | |
| | | | | | | | | | NO: 2547)(1) | | | | |
| | | | | | | | | | DEGDKWR | GST(36) | G80(58) | | |
| | | | | | | | | | (SEQ ID NO: | | | | |
| | | | | | | | | | 2573)(1)- | | | | |
| | | | | | | | | | QAPGKER | | | | |
| | | | | | | | | | (SEQ ID NO: | | | | |
| | | | | | | | | | 2574)(5) | | | | |
| | | | | | | | | G81 | / | / | / | / | / |
| | | | | | | | | G82 | DNAKYTTYL | GST(36) | G82(90) | Seq_ | E2 |
| | | | | | | | | | QMNSLKPED | | | 1411 | |
| | | | | | | | | | TAVYYCNVR | | | | |
| | | | | | | | | | (SEQ ID NO: | | | | |
| | | | | | | | | | 2578)(4)- | | | | |
| | | | | | | | | | DEGDKWR | | | | |
| | | | | | | | | | (SEQ ID NO: | | | | |
| | | | | | | | | | 2573)(1) | | | | |
| | | | | | | | | | DPAHVQLVE | GST(113) | G82(13) | | |
| | | | | | | | | | SGGGLVQAG | | | | |
| | | | | | | | | | GSLR (SEQ ID | | | | |
| | | | | | | | | | NO: 2579)(1)- | | | | |
| | | | | | | | | | IAYSKDFETL | | | | |
| | | | | | | | | | KVDFLSK | | | | |
| | | | | | | | | | (SEQ ID NO: | | | | |
| | | | | | | | | | 2580)(5) | | | | |
| | | | | | | | | | DPAHVQLVE | GST(119) | G82(13) | | |
| | | | | | | | | | SGGGLVQAG | | | | |
| | | | | | | | | | GSLR (SEQ ID | | | | |
| | | | | | | | | | NO: 2579)(1)- | | | | |
| | | | | | | | | | DFETLKVDF | | | | |
| | | | | | | | | | LSK (SEQ ID | | | | |
| | | | | | | | | | NO: 2550)(6) | | | | |
| | | | | | | | | | KFELGLEFPN | GST(51) | G82(58) | | |
| | | | | | | | | | LPYYIDGDV | | | | |
| | | | | | | | | | K (SEQ ID | | | | |
| | | | | | | | | | NO: 2581)(7)- | | | | |
| | | | | | | | | | QAPGKQR(SEQ | | | | |
| | | | | | | | | | ID NO: | | | | |
| | | | | | | | | | 2551)(5) | | | | |
| | | | | | | | | G83 | DPAQVQLVE | GST(194) | G83(13) | Seq_ | E1 |
| | | | | | | | | | SGGGLVQAG | | | 22759 | |
| | | | | | | | | | GSLR (SEQ ID | | | | |
| | | | | | | | | | NO: 2582)(1)- | | | | |
| | | | | | | | | | YLKSSK (SEQ | | | | |
| | | | | | | | | | ID NO: | | | | |
| | | | | | | | | | 2557)(3) | | | | |
| | | | | | | | | | DPAQVQLVE | GST(191) | G83(13) | | |
| | | | | | | | | | SGGGLVQAG | | | | |
| | | | | | | | | | GSLR (SEQ ID | | | | |
| | | | | | | | | | NO: 2582)(1)- | | | | |
| | | | | | | | | | RIEAIPQIDK | | | | |
| | | | | | | | | | YLK (SEQ ID | | | | |
| | | | | | | | | | NO: 2537)(10) | | | | |
| | | | | | | | | G84 | / | / | / | / | / |
| | | | | | | | | G85 | / | / | / | / | / |
| | | | | | | | | G86 | / | / | / | / | / |
| | | | | | | | | G87 | / | / | / | / | / |
| | | | | | | | | G88 | / | / | / | / | / |
| | | | | | | | | G89 | / | / | / | / | / |
| | | | | | | | | G90 | MASMTGGQ | GST(2) | G90(13) | Seq_ | E3 |
| | | | | | | | | | QMGRDPAD | | | 13998 | |
| | | | | | | | | | VQLVESGGG | | | | |
| | | | | | | | | | LVQAGGSLR | | | | |
| | | | | | | | | | (SEQ ID NO: | | | | |
| | | | | | | | | | 2583)(13)- | | | | |
| | | | | | | | | | SPILGYWK | | | | |
| | | | | | | | | | (SEQ ID NO: | | | | |
| | | | | | | | | | 2584)(1) | | | | |
| | | | | | | | | G91 | / | / | / | / | / |
| | | | | | | | | G92 | / | / | / | / | / |
| | | | | | | | | G93 | / | / | / | / | / |
| | | | | | | | | G94 | / | / | / | / | / |
| | | | | | | | | G95 | / | / | / | / | / |
| | | | | | | | | G96 | DPADVQLVE | GST(40) | G96(13) | Seq_ | E1 |
| | | | | | | | | | SGGGLVQAG | | | 17861 | |
| | | | | | | | | | GSLK (SEQ ID | | | | |
| | | | | | | | | | NO: 2585)(1)- | | | | |
| | | | | | | | | | YEEHLYERD | | | | |
| | | | | | | | | | EGDKWR | | | | |
| | | | | | | | | | (SEQ ID NO: | | | | |
| | | | | | | | | | 2539)(13) | | | | |
| | | | | | | | | | DPADVQLVE | GST(40) | G96(16) | | |
| | | | | | | | | | SGGGLVQAG | | | | |
| | | | | | | | | | GSLK (SEQ ID | | | | |
| | | | | | | | | | NO: 2585)(4)- | | | | |
| | | | | | | | | | YEEHLYERD | | | | |
| | | | | | | | | | EGDKWR | | | | |
| | | | | | | | | | (SEQ ID NO: | | | | |
| | | | | | | | | | 2539)(13) | | | | |
| | | | | | | | | | LLLEYLEEK | GST(27) | G96(13) | | |
| | | | | | | | | | YEEHLYERD | | | | |
| | | | | | | | | | EGDK (SEQ | | | | |
| | | | | | | | | | ID NO: | | | | |
| | | | | | | | | | 2586)(9)- | | | | |
| | | | | | | | | | DPADVQLVE | | | | |
| | | | | | | | | | SGGGLVQAG | | | | |
| | | | | | | | | | GSLK (SEQ ID | | | | |
| | | | | | | | | | NO: 2585)(1) | | | | |
| | | | | | | | | | DPADVQLVE | GST(9) | G96(13) | | |
| | | | | | | | | | SGGGLVQAG | | | | |
| | | | | | | | | | GSLK (SEQ ID | | | | |
| | | | | | | | | | NO: 2585)(1)- | | | | |
| | | | | | | | | | MSPILGYWKI | | | | |
| | | | | | | | | | K(SEQ ID NO: | | | | |
| | | | | | | | | | 2587)(9) | | | | |
| | | | | | | | | G97 | / | / | / | / | / |
| | | | | | | | | G98 | / | / | / | / | / |

[symbol]

indicates data missing or illegible when filed

TABLE 2
Summary of HSA Nbs and their biophysical and physiochemical properties.

| | | | | | | | | ELISA | | | | | |
|---|---|---|---|---|---|---|---|---|---|---|---|---|---|
| | | | | | | | | affinity | | | | SPR | |
| | | | | salt | lowpH | highpH | | (LogIC50 | Mutant | SPR ka | SPR kd | KD | Cross- |
| ID | Enzyme | Protein Sequence | SEQ ID NO | trend | trend | trend | Soluble | (oD450nm)) | Screening | (1/Ms) | (1/s) | (M) | linker |
| H1 | Trypsin | MASMTGGQQMGRDPGSS | SEQ ID NO: | 2 | 1 | 2 | Yes | 4.916 | / | 9.73E+06 | 1.19E−03 | 1.22E−09 | DSS |
| | | SGSMAQVQLVESGGGLV | 99 | | | | | | | | | | |
| | | QPGGSLRLSCVASGIMFDI | | | | | | | | | | | |
| | | YTMRWYRQAPGKQRELV | | | | | | | | | | | |
| | | AAITGAGRANYNDDSVK | | | | | | | | | | | |
| | | GRFTISRDNAKNTVYLQM | | | | | | | | | | | |
| | | NRMKPEDTALYECNTEIL | | | | | | | | | | | |
| | | GGGPNYWGRGTQVTVSE | | | | | | | | | | | |
| | | PKTPKGGKGGGLEHHHH | | | | | | | | | | | |
| | | HH | | | | | | | | | | | |
| H2 | Trypsin/ | MASMTGGQQMGRDPENL | SEQ ID NO: | 2 | / | 2 | Yes | 5.883 | Decreased | 2.34E+05 | 3.99E−05 | 1.70E−10 | DSS |
| | Chymo | YFQGAQVQLVESGGGLV | 100 | | | | | | | | | | |
| | | QAGGSLRLSCTASGRTFTP | | | | | | | | | | | |
| | | YTIGWFRQAPGKEREFVA | | | | | | | | | | | |
| | | SILWSGINTDYADSVKGRF | | | | | | | | | | | |
| | | AISRDNAKNAAYLQMSNL | | | | | | | | | | | |
| | | KPEDTAVYYCATGGGLG | | | | | | | | | | | |
| | | YYRSVSQYDYWGQGTQV | | | | | | | | | | | |
| | | TVSEPKTPKGGKGGGLEH | | | | | | | | | | | |
| | | HHHHH | | | | | | | | | | | |
| H3 | Chymo | MASMTGGQQMGRDPENL | SEQ ID NO: | 1 | 0 | 2 | Yes | 7 | Decreased | 1.11E+06 | 5.04E−04 | 4.54E−10 | EDC |
| | | YFQGAQVQLVESGGGLV | [symbol] | | | | | | | | | | |
| | | QAGGSLRLSCVASGRTFE | | | | | | | | | | | |
| | | PFVMGWFRQAPGKEREFV | | | | | | | | | | | |
| | | ATISWSGGSLSYADSVKG | | | | | | | | | | | |
| | | RFTVSRDNAKNTVYLQM | | | | | | | | | | | |
| | | NSLKPEDTAVYYCAAAPG | | | | | | | | | | | |
| | | VGNYRYTFQYDYWGQGT | | | | | | | | | | | |
| | | QVTVSEPKTPKGGKGGGL | | | | | | | | | | | |
| | | EHHHHHH | | | | | | | | | | | |
| H4 | Trypsin | MASMTGGQQMGRDPNSA | SEQ ID NO: | 0 | 0 | 2 | Yes | 5.4 | No change | 9.92E+05 | 2.64E−04 | 2.66E−10 | DSS |
| | | HVQLVESGGGLVQTGGSL | 102 | | | | | | | | | | |
| | | RLACAASGRAFSTYAMG | | | | | | | | | | | |
| | | WFRQAPGKEREFVASINR | | | | | | | | | | | |
| | | SGSSTYYADSVKGRFTISR | | | | | | | | | | | |
| | | DNGKDTVYLQMNRLIPED | | | | | | | | | | | |
| | | TAVYYCAADSEGVGFRN | | | | | | | | | | | |
| | | MLEYDYWGQGTQVTVSS | | | | | | | | | | | |
| | | EPKTPKGGCGGGAAALEH | | | | | | | | | | | |
| | | HHHHH | | | | | | | | | | | |
| H5 | Chymo | MASMTGGQQMGRDPNSA | SEQ ID NO: | 1 | 0 | 2 | No | / | / | / | | | / |
| | | EVQLVESGGGLVQAGGSL | | | | | | | | | | | |
| | | RLSCAASGRTFIPYTTGWF | | | | | | | | | | | |
| | | RQTPGKEREFVATITWSGI | | | | | | | | | | | |
| | | STKYADSVKGRFTISRDN | | | | | | | | | | | |
| | | AKNTVYLQMNSLKPEDT | | | | | | | | | | | |
| | | AVYYCTKNPRALALNRD | | | | | | | | | | | |
| | | YWGQGTQVTVSSEPKTPK | | | | | | | | | | | |
| | | GGCGGGAAALEHHHHHH | | | | | | | | | | | |
| H6 | Chymo | MASMTGGQQMGRDPNSA | SEQ ID NO: | 2 | 1 | / | Yes | 2.905 | No change | / | | | DSS |
| | | EVQLVESGGGLVQVGGSL | 104 | | | | | | | | | | |
| | | TLSCAAAGSTFTTNAMA | | | | | | | | | | | |
| | | WFRQFPGKERELVAAISW | | | | | | | | | | | |
| | | GGLGYVADSVRGRFTISR | | | | | | | | | | | |
| | | PTKNMMILQLNSLEREDT | | | | | | | | | | | |
| | | AIYYCAARKMSTVATEAT | | | | | | | | | | | |
| | | MYAYWGHGTQVTVSSEP | | | | | | | | | | | |
| | | KTPKGGCGGGAAALEHH | | | | | | | | | | | |
| | | HHHH | | | | | | | | | | | |
| H7 | Chymo | MASMTGGQQMGRDPNSA | SEQ ID NO: | 1 | 0 | 2 | Yes | 5.621 | No change | 9.35E+04 | 9.79E−05 | 1.09E−09 | DSS |
| | | DVQLVESGGGSVQAGGSL | 105 | | | | | | | | | | |
| | | RLSCAASGGTFSSYAMGW | | | | | | | | | | | |
| | | YRQAPGKEREFVSGISWS | | | | | | | | | | | |
| | | GSSIDYVDSVKGRFTISRD | | | | | | | | | | | |
| | | NAKNTVYLQMNSLKPED | | | | | | | | | | | |
| | | TAVYYCGAADPMGLGYG | | | | | | | | | | | |
| | | LGPRPVDRLLSAECDYWG | | | | | | | | | | | |
| | | QGTQVTVSSEPKTPKGGC | | | | | | | | | | | |
| | | GGAAALEHHHHHH | | | | | | | | | | | |
| H8 | Chymo | MASMTGGQQMGRDPNSA | SEQ ID NO: | / | 0 | 2 | No | / | / | / | | | / |
| | | DVQLVESGGGLVQAGGSL | 106 | | | | | | | | | | |
| | | RLSCAASGRTFSSYAMGW | | | | | | | | | | | |
| | | FRQAPGKEREFVSAISRSG | | | | | | | | | | | |
| | | GSTYYTDSVKGRFTISRDN | | | | | | | | | | | |
| | | AKNTVYLQMNSLKPEDT | | | | | | | | | | | |
| | | AVYYCAAAEGLASGSYD | | | | | | | | | | | |
| | | YTPPLKSSWYDYWGQGT | | | | | | | | | | | |
| | | QVTVSSEPKTPKGGCGGA | | | | | | | | | | | |
| | | AALEHHHHHH | | | | | | | | | | | |
| H9 | Trypsin | MASMTGGQQMGRDPNSA | SEQ ID NO: | 2 | / | 2 | No | / | / | / | | | / |
| | | EVQLVESGGGLVQAGGSL | 107 | | | | | | | | | | |
| | | RLSCVASGRTFSYRAMG | | | | | | | | | | | |
| | | WFHQAPGKEREFVAAVG | | | | | | | | | | | |
| | | SSGLTTYYADSVKGRFTIS | | | | | | | | | | | |
| | | RDNAKNTVYLQMNSLQL | | | | | | | | | | | |
| | | EDTAVYYCAAAKFGYVV | | | | | | | | | | | |
| | | VTAKEYEYWGQGTQVTV | | | | | | | | | | | |
| | | SSEPKTPKGGCGGAAALE | | | | | | | | | | | |
| | | HHHHHH | | | | | | | | | | | |
| H10 | Chymo | MASMTGGQQMGRDPNSA | SEQ ID NO: | 2 | / | / | Yes | 1.462 | / | / | | | / |
| | | DVQLVESGGGLVQAGGSL | 108 | | | | | | | | | | |
| | | RLSCRASGLPFGPYTMGW | | | | | | | | | | | |
| | | FRQTPGQEREFVAAITWSS | | | | | | | | | | | |
| | | MNTNYADSVKGRFTISRD | | | | | | | | | | | |
| | | SAKNTVYLQMNTLKPDD | | | | | | | | | | | |
| | | TAVYYCAADDRAVPMLG | | | | | | | | | | | |
| | | DFEDYIYWGQGTQVTVSS | | | | | | | | | | | |
| | | EPKTPKGGCGGAAALEHH | | | | | | | | | | | |
| | | HHHH | | | | | | | | | | | |
| H11 | Trypsin | MASMTGGQQMGRDPNSA | SEQ ID NO: | 2 | / | 2 | Yes | 6.272 | No change | 2.55E+05 | 1.55E−05 | 6.10E−11 | DSS |
| | | QVQLVESGGGLVQVGGSL | 109 | | | | | | | | | | |
| | | RLSCAASGRTFSNYVMG | | | | | | | | | | | |
| | | WFRQAPGKEREFVAYIHW | | | | | | | | | | | |
| | | SGSSTSYADSVKGRFTISR | | | | | | | | | | | |
| | | DNTKNTMYLQMNSLKPE | | | | | | | | | | | |
| | | DTAVYYCTADQYASTLLR | | | | | | | | | | | |
| | | AAGEYWGQGTQVTVSSE | | | | | | | | | | | |
| | | PKTPKGGCGGAAALEHH | | | | | | | | | | | |
| | | HHHH | | | | | | | | | | | |
| H12 | Chymo | MASMTGGQQMGRDPNSA | SEQ ID NO: | 2 | 0 | / | Yes | 1.617 | No change | / | | | / |
| | | HVQLVESGGGLVQAGGSL | 110 | | | | | | | | | | |
| | | RLSCVSSGRTYRWNAMG | | | | | | | | | | | |
| | | WFRQAPGKEREFVAAIDW | | | | | | | | | | | |
| | | DGRNTDYADSVKGRFTIS | | | | | | | | | | | |
| | | RDNAKNTVFLQMNRLKS | | | | | | | | | | | |
| | | EDTAVYSCALDRVVITSM | | | | | | | | | | | |
| | | RTNFDVWGQGTQVTVSSE | | | | | | | | | | | |
| | | PKTPKGGCGGAAALEHH | | | | | | | | | | | |
| | | HHHH | | | | | | | | | | | |
| H13 | Trypsin | MASMTGGQQMGRDPNSA | SEQ ID NO: | / | / | 2 | Yes | 1.962 | / | / | | | / |
| | | HVQLVESGGGLVQAGGSL | 111 | | | | | | | | | | |
| | | RLSCAASGRTFSTYHMGW | | | | | | | | | | | |
| | | FRQAPGKAREFVAAITGS | | | | | | | | | | | |
| | | GGITYYADSVKGRFTISRD | | | | | | | | | | | |
| | | NAKNTVYLQMNSLKPED | | | | | | | | | | | |
| | | TAVYYCAADTRAYGLVPS | | | | | | | | | | | |
| | | TTSSRYNYWGQGTQVTVS | | | | | | | | | | | |
| | | SEPKTPKGGCGGAAALEH | | | | | | | | | | | |
| | | HHHHH | | | | | | | | | | | |
| H14 | Trypsin | MASMTGGQQMGRDPNSA | SEQ ID NO: | 2 | 1 | 2 | Yes | 5.964 | Decresed | 2.50E+05 | 5.57E−06 | 2.23E−11 | / |
| | | HVQLVESGGGLVQAGGSL | 112 | | | | | | | | | | |
| | | RLSCTASGRTFTPYTMGW | | | | | | | | | | | |
| | | FRQAPGKEREFVASILWS | | | | | | | | | | | |
| | | GNNRDYADSVKDRFAISR | | | | | | | | | | | |
| | | DNAKNTAYLQMNSLKPE | | | | | | | | | | | |
| | | DTAVYYCAAGDGLGFYR | | | | | | | | | | | |
| | | SVNQYDYWGQGTQVTVS | | | | | | | | | | | |
| | | SEPKTPKGGCGGAAALEH | | | | | | | | | | | |
| | | HHHHH | | | | | | | | | | | |
| H15 | Trypsin | MASMTGGQQMGRDPNSA | SEQ ID NO: | 2 | 0 | 0 | Yes | No | / | / | | | / |
| | | QVQLVESGGGLVQAGDSL | 113 | | | | | binding | | | | | |
| | | RLSCAASERTSNYAMGWF | | | | | | | | | | | |
| | | RQAPGKEREFVADINHTG | | | | | | | | | | | |
| | | GRRKYGDSVKGRFTISRD | | | | | | | | | | | |
| | | NAENMVYLQMNNLQVED | | | | | | | | | | | |
| | | TAVYYCATGLRYDVSGY | | | | | | | | | | | |
| | | APDYRYWGRGTQVTVSS | | | | | | | | | | | |
| | | EPKTPKGGCGGAAALEHH | | | | | | | | | | | |
| | | HHHH | | | | | | | | | | | |
| H16 | Chymo | MASMTGGQQMGRDPNSA | SEQ ID NO: | / | 0 | 2 | Yes | 5.986 | No change | 1.34E+06 | 1.16E−04 | 8.62E−11 | / |
| | | QVQLVESGGGLVQTGGSL | 114 | | | | | | | | | | |
| | | TLSCAASGRTFSTKSMGW | | | | | | | | | | | |
| | | FRQAPGKEREFVADINWN | | | | | | | | | | | |
| | | GGITHYADSVEGRFTISRD | | | | | | | | | | | |
| | | NANDMVYLQMNSLKPED | | | | | | | | | | | |
| | | TAVYYCAGGRYSTLFSKS | | | | | | | | | | | |
| | | EADYDYWGQGTQVTVSS | | | | | | | | | | | |
| | | EPKTPKGGCGGAAALEHH | | | | | | | | | | | |
| | | HHHH | | | | | | | | | | | |
| H17 | Trypsin | MASMTGGQQMGRDPNSA | SEQ ID NO: | 2 | 0 | 2 | No | / | / | / | | | / |
| | | QVQLVESGGGLAQAGGSL | 115 | | | | | | | | | | |
| | | RLSCAASGGTFSNSCMGW | | | | | | | | | | | |
| | | FRQAPGMEREFVVIIRSTG | | | | | | | | | | | |
| | | HTTYADSVEGRFTVSREIA | | | | | | | | | | | |
| | | KNTVYLEMNSLKPEDTAV | | | | | | | | | | | |
| | | YVCAAGVSDYGCYRTSGI | | | | | | | | | | | |
| | | NYWGQGTQVTVSSEPKTP | | | | | | | | | | | |
| | | KGGCGGAAALEHHHHHH | | | | | | | | | | | |
| H18 | Trypsin | MASMTGGQQMGRDPNSA | SEQ ID NO: | 23 | 2 | 2 | Yes | 5.646 | Decreased | 1.71E+05 | 8.71E−05 | 5.02E−10 | DSS |
| | | EVQLVESGGGLVQAGGSL | 116 | | | | | | | | | | |
| | | RLSCTASGPKDTPYTMGW | | | | | | | | | | | |
| | | FRQVPGKEREFVASVLWS | | | | | | | | | | | |
| | | GINTDYADSVKGRFAISRN | | | | | | | | | | | |
| | | NAKNTMYLQMNSLKPED | | | | | | | | | | | |
| | | TAVYYCAAGYGLGFYRSI | | | | | | | | | | | |
| | | SQYDYWGHGTQVTVSSEP | | | | | | | | | | | |
| | | KTPKGGCGGAAALEHHH | | | | | | | | | | | |
| | | HHH | | | | | | | | | | | |
| H19 | Trypsin | MASMTGGQQMGRDPNSA | SEQ ID NO: | 2 | 2 | 2 | No | / | / | / | | | / |
| | | HVQLVESGGGLVQAGGSL | 117 | | | | | | | | | | |
| | | RLSCTASGPKDTPYTMGW | | | | | | | | | | | |
| | | FRQVPGKEREFVASVLWS | | | | | | | | | | | |
| | | GINTDYADSVKGRFAISRN | | | | | | | | | | | |
| | | NAKNTMYLQMNSLKPED | | | | | | | | | | | |
| | | TAVYYCAAGYGLGFYRS | | | | | | | | | | | |
| | | VSQHDYWGHGTQVTVSS | | | | | | | | | | | |
| | | EPKTPKGGCGGAAALEHH | | | | | | | | | | | |
| | | HHHH | | | | | | | | | | | |
| H20 | Trypsin | MASMTGGQQMGRDPNSA | SEQ ID NO: | 2 | 2 | 2 | Yes | 5.759 | Decreased | 1.54E+05 | 3.80E−05 | 2.47E−10 | DSS |
| | | EVQLVESGGGLVQAGGSL | 118 | | | | | | | | | | |
| | | RLSCTASGPKDTPYTMGW | | | | | | | | | | | |
| | | FRQVPGKEREFVASVLWS | | | | | | | | | | | |
| | | GINTDYADSVKGRFAISRN | | | | | | | | | | | |
| | | NAKNTMYLQMNSLKPED | | | | | | | | | | | |
| | | TAVYYCAAGYGLGFYRT | | | | | | | | | | | |
| | | VSQYDYWGHGTQVTVSS | | | | | | | | | | | |
| | | EPKTPKGGCGGAAALEHH | | | | | | | | | | | |
| | | HHHH | | | | | | | | | | | |
| | | | | | | | | | | | | | EDC |
| H21 | Trypsin | MASMTGGQQMGRDPNSA | SEQ ID NO: | 1 | 0 | 2 | Yes | 5.628 | No change | 6.83E+05 | 1.82E−04 | 2.66E−10 | DSS |
| | | QVQLVESGGGLVQAGGSL | 119 | | | | | | | | | | |
| | | RLSCAASGYTSGNDAMG | | | | | | | | | | | |
| | | WFRQAPGKEREFVGAIRW | | | | | | | | | | | |
| | | SGVSTYYADSVKGRFTISR | | | | | | | | | | | |
| | | DGAKNTLYLQMNSLKPE | | | | | | | | | | | |
| | | DTAVYYCAAKFTGSAWY | | | | | | | | | | | |
| | | GVQKLESTYWDYWGQGT | | | | | | | | | | | |
| | | QVTVSSEPKTPKGGCGGA | | | | | | | | | | | |
| | | AALEHHHHHH | | | | | | | | | | | |
| H22 | Trypsin | MASMTGGQQMGRDPNSA | SEQ ID NO: | 1 | 0 | 2 | Yes | 4.211 | No change | / | | | DSS |
| | | HVQLVESGGGLVQAGGSL | 120 | | | | | | | | | | |
| | | RLSCTASARTSNAMGWFR | | | | | | | | | | | |
| | | RAPGKERDFVAAISESGRT | | | | | | | | | | | |
| | | TDYADSVKGRFTISRDTA | | | | | | | | | | | |
| | | KNTVYLQMISLKPEDTAV | | | | | | | | | | | |
| | | YYCARKRVADAISSNYEF | | | | | | | | | | | |
| | | RYDYWGQGTQVTVSSEP | | | | | | | | | | | |
| | | KTPKGGCGGAAALEHHH | | | | | | | | | | | |
| | | HHH | | | | | | | | | | | |
| H23 | Chymo | MASMTGGQQMGRDPNSA | SEQ ID NO: | 1 | 0 | 2 | Yes | 1.625 | / | / | | | / |
| | | DVQLVESGGGLVQAGGSL | 121 | | | | | | | | | | |
| | | TLSCAASGRTFSSSTMGW | | | | | | | | | | | |
| | | FRRAPGKEREFVAAISGSA | | | | | | | | | | | |
| | | RTTDYADSVKGRFTISRD | | | | | | | | | | | |
| | | NAKNTVYLQMISLKPEDT | | | | | | | | | | | |
| | | AIYYCARKRVVDVTTSNY | | | | | | | | | | | |
| | | ELRYDYWGQGTQVTVSS | | | | | | | | | | | |
| | | EPKTPKGGCGGAAALEHH | | | | | | | | | | | |
| | | HHHH | | | | | | | | | | | |
| H24 | Chymo | MASMTGGQQMGRDPNSA | SEQ ID NO: | 2 | 2 | / | Yes | No | / | / | | | / |
| | | QVQLVESGGGLVQAGGSL | 122 | | | | | binding | | | | | |
| | | RLSCVSSGRTYRWNAMG | | | | | | | | | | | |
| | | WFRQAPGKEREFVAAIDW | | | | | | | | | | | |
| | | DGRNTDYADSVKGRFTIS | | | | | | | | | | | |
| | | RDNAKNTVYLQMNSLKV | | | | | | | | | | | |
| | | EDTAIYYCAAREWGSGGY | | | | | | | | | | | |
| | | SSIASYAYWGQGTQVTVS | | | | | | | | | | | |
| | | SEPKTPKGGCGGAAALEH | | | | | | | | | | | |
| | | HHHHH | | | | | | | | | | | |
| H25 | Trypsin | MASMTGGQQMGRDPNSA | SEQ ID NO: | 1 | / | 2 | Yes | 5.344 | No change | 9.36E+05 | 8.02E−04 | 8.58E−10 | DSS |
| | | DVQLVESGGGLVQAGGSL | 123 | | | | | | | | | | |
| | | RLSCAASGRTISDYGMAW | | | | | | | | | | | |
| | | FRQAPGKEREFVGVITSNS | | | | | | | | | | | |
| | | VTTYYADSVKGRFTISRD | | | | | | | | | | | |
| | | NTKNTVYLQMISLKPEDT | | | | | | | | | | | |
| | | AIYYCAARIPVGFYYNAR | | | | | | | | | | | |
| | | NYDFWGQGTQVTVSSEPK | | | | | | | | | | | |
| | | TPKGGCGGAAALEHHHH | | | | | | | | | | | |
| | | HH | | | | | | | | | | | |
| H26 | Chymo | MASMTGGQQMGRDPNSA | SEQ ID NO: | 1 | 0 | 2 | Yes | 2.813 | No change | / | | | DSS |
| | | QVQLVESGGGLVQAGGSL | 124 | | | | | | | | | | |
| | | RLSCAASGRTPYVMGWF | | | | | | | | | | | |
| | | RQAPGNEREFVASISWTY | | | | | | | | | | | |
| | | GYTNYANSVKGRFRISKD | | | | | | | | | | | |
| | | NAKNTVLLQMNSLKPEDT | | | | | | | | | | | |
| | | AVYYCAARRGEDPEYDY | | | | | | | | | | | |
| | | WGQGTQVTVSSEPKTPKG | | | | | | | | | | | |
| | | GCGGAAALEHHHHHH | | | | | | | | | | | |
| H27 | Chymo | MASMTGGQQMGRDPNSA | SEQ ID NO: | 2 | / | 0 | Yes | 2.575 | No change | / | | | DSS |
| | | HVQLVESGGGLVQAGGSL | 125 | | | | | | | | | | |
| | | RLSCIASGRTFSTYHMGW | | | | | | | | | | | |
| | | FREAPGKGREFVAAITQN | | | | | | | | | | | |
| | | GGTTYYADSVKGRFTISR | | | | | | | | | | | |
| | | DNAKNTVYLQMGSLKPE | | | | | | | | | | | |
| | | DTAVYYCAASPALIGRIYF | | | | | | | | | | | |
| | | GNENYSWGQGTQVTVSS | | | | | | | | | | | |
| | | EPKTPKGGCGGAAALEHH | | | | | | | | | | | |
| | | HHHH | | | | | | | | | | | |
| H28 | Trypsin | MASMTGGQQMGRDPNSA | SEQ ID NO: | 2 | / | / | Yes | 5.116 | No change | 5.24E+06 | 8.68E−03 | 1.66E−09 | DSS |
| | | DVQLVESGGGLAQAGGSL | 126 | | | | | | | | | | |
| | | RLSCAASGRTFSNECMGW | | | | | | | | | | | |
| | | FRQAPGKEREFVATIRSTG | | | | | | | | | | | |
| | | HISYATSVQGRFTVSRDIA | | | | | | | | | | | |
| | | KNTVYLEMNNLKPEDTA | | | | | | | | | | | |
| | | VYSCGAGVSDYGCYRTSG | | | | | | | | | | | |
| | | YNYWGQGTQVTVSSEPK | | | | | | | | | | | |
| | | TPKGGCGGAAALEHHHH | | | | | | | | | | | |
| | | HH | | | | | | | | | | | |
| H29 | Chymo | MASMTGGQQMGRDPNSA | SEQ ID NO: | 2 | 2 | 1 | Yes | 1.862 | No change | / | | | / |
| | | QVQLVESGGGLVPAGGSL | 127 | | | | | | | | | | |
| | | RLSCAASGRTFSLYRMGW | | | | | | | | | | | |
| | | FRQAPGKEREFVAAIIWSS | | | | | | | | | | | |
| | | GSTYYADSVKGRFTISRDI | | | | | | | | | | | |
| | | AKNTVYLEMNSLKPEDTA | | | | | | | | | | | |
| | | VYSCGAGVSDYGCYRTSG | | | | | | | | | | | |
| | | YAYWGQGTQVTVSSEPK | | | | | | | | | | | |
| | | TPKGGCGGAAALEHHHH | | | | | | | | | | | |
| | | HH | | | | | | | | | | | |
| H30 | Trypsin | MASMTGGQQMGRDPNSA | SEQ ID NO: | / | 0 | 2 | Yes | 5.895 | No change | 1.03E+06 | 1.68E−04 | 1.64E−10 | / |
| | | HVQLVESGGGLAQAGGSL | 128 | | | | | | | | | | |
| | | RLSCAASGGTFSNSCMGW | | | | | | | | | | | |
| | | FRQAPGMEREFVAIIRSTG | | | | | | | | | | | |
| | | HTTYADSVEGRFTVSRDI | | | | | | | | | | | |
| | | AKNTVYLEMNSLKPEDTA | | | | | | | | | | | |
| | | VYSCVAGVSDYGCYRTSG | | | | | | | | | | | |
| | | IKYWGQGTQVTVSSEPKT | | | | | | | | | | | |
| | | PKGGCGGAAALEHHHHH | | | | | | | | | | | |
| | | H | | | | | | | | | | | |
| H31 | Chymo | MASMTGGQQMGRDPNSA | SEQ ID NO: | 0 | 0 | 0 | Yes | 1.075 | / | / | | | / |
| | | QVQLVESGGGLVQPGGSL | 129 | | | | | | | | | | |
| | | RLSCTPSGFRLEDYPIAWF | | | | | | | | | | | |
| | | RQAPGKEREGLSCITSGDG | | | | | | | | | | | |
| | | RTYYEESVKGRFTISRDNA | | | | | | | | | | | |
| | | QNKVYLQMNKLTPEDTA | | | | | | | | | | | |
| | | VYHCATVPSDNLCGYLHR | | | | | | | | | | | |
| | | RPFASWGQGTQVTVSSEP | | | | | | | | | | | |
| | | KTPKGGCGGGAAALEHH | | | | | | | | | | | |
| | | HHHH | | | | | | | | | | | |
| H32 | Chymo | MASMTGGQQMGRDPNSA | SEQ ID NO: | / | 0 | 0 | Yes | 5.335 | / | / | | | / |
| | | HVQLVESGGGLVQAGGSL | 130 | | | | | | | | | | |
| | | RLSCAASDTIDNYARAWF | | | | | | | | | | | |
| | | RQAPGKEREFVAAITWTF | | | | | | | | | | | |
| | | GTPYYTDSVKGRFTISRDD | | | | | | | | | | | |
| | | AKNTVYLQMNSLKPEDT | | | | | | | | | | | |
| | | AVYYCAASLYLPVRTASG | | | | | | | | | | | |
| | | GYRLDTDRPQYWGQGTQ | | | | | | | | | | | |
| | | VTVSSEPKTPKGGCGGGA | | | | | | | | | | | |
| | | AALEHHHHHH | | | | | | | | | | | |
| H33 | Trypsin | MASMTGGQQMGRDPNSA | SEQ ID NO: | 1 | 0 | 0 | Yes | 5.033 | / | / | | | DSS |
| | | HVQLVESGGGLVQAGGSL | [symbol] | | | | | | | | | | |
| | | RLSCAASGRTLSSYDMGW | | | | | | | | | | | |
| | | FRQPPGKEREFVAAITRHD | | | | | | | | | | | |
| | | FNTFYRDSVKGRFTISRDN | | | | | | | | | | | |
| | | AKNTVYLQMNSLKSEDT | | | | | | | | | | | |
| | | AVYFCAARLDPIFASNSEY | | | | | | | | | | | |
| | | APLYDYWGQGTQVTVSS | | | | | | | | | | | |
| | | EPKTPKGGCGGGAAALEH | | | | | | | | | | | |
| | | HHHHH | | | | | | | | | | | |
| H34 | Chymo | MASMTGGQQMGRDPNSA | SEQ ID NO: | 0 | 0 | 0 | Yes | 5.065 | / | / | | | DSS |
| | | QVQLVESGGGLVQAGGSL | 132 | | | | | | | | | | |
| | | RLSCAASGRTLSSYDMGW | | | | | | | | | | | |
| | | FRKAPGKEREFVAAITRH | | | | | | | | | | | |
| | | DYNTYYRDSVKGRFTISR | | | | | | | | | | | |
| | | DNAKNTVYLQMNSLKSE | | | | | | | | | | | |
| | | DTAVYFCAARLDPIFASNS | | | | | | | | | | | |
| | | AYSNLYDYWGQGTQVTV | | | | | | | | | | | |
| | | SSEPKTPKGGCGGGAAAL | | | | | | | | | | | |
| | | EHHHHHH | | | | | | | | | | | |
| H35 | Trypsin | MASMTGGQQMGRDPNSA | SEQ ID NO: | 0 | / | 0 | Yes | No | / | / | | | / |
| | | EVQLVESGGGLVQAGGSL | 133 | | | | | binding | | | | | |
| | | RVSCAVSGISIYHSGWYR | | | | | | | | | | | |
| | | QAPGKERELVAGISRGGS | | | | | | | | | | | |
| | | TNYADSVKGRFTISRDSGE | | | | | | | | | | | |
| | | NTVYLQMNSLKPEDTAV | | | | | | | | | | | |
| | | YYCKIDWDYRGVSQTAW | | | | | | | | | | | |
| | | GQGTQVTVSSEPKTPKGG | | | | | | | | | | | |
| | | CGGGAAALEHHHHHH | | | | | | | | | | | |
| H36 | Chymo | MASMTGGQQMGRDPNSA | SEQ ID NO: | 0 | 1 | 0 | No | / | / | / | | | / |
| | | EVQLVESGGGLVQAGGSL | 134 | | | | | | | | | | |
| | | RLSCAAPAIALADYAIGW | | | | | | | | | | | |
| | | FRQGPGKEREGISCVASET | | | | | | | | | | | |
| | | DTTRYADSVKGRFTISRD | | | | | | | | | | | |
| | | NAKNLVYLQMNSLKPDD | | | | | | | | | | | |
| | | TAVYYCATEVMECRGLS | | | | | | | | | | | |
| | | YNAWGSWGQGTQVTVSS | | | | | | | | | | | |
| | | EPKTPKGGCGGGAAALEH | | | | | | | | | | | |
| | | HHHHH | | | | | | | | | | | |
| H37 | Chymo | MASMTGGQQMGRDPNSA | SEQ ID NO: | 2 | 0 | 1 | Yes | 1.49 | / | / | | | / |
| | | QVQLVESGGGLVQAGGSL | 135 | | | | | | | | | | |
| | | RLSCAASGLTFSNYALGW | | | | | | | | | | | |
| | | FRRAPGKERDFVAAISYSG | | | | | | | | | | | |
| | | GSTDYADSVKGRFTISRD | | | | | | | | | | | |
| | | NAKNTVYLQMNSLKPED | | | | | | | | | | | |
| | | TAVYYCAAAYLGWGTAR | | | | | | | | | | | |
| | | TAYEYWGQGTQVTVSSEP | | | | | | | | | | | |
| | | KTPKGGCGGGAAALEHH | | | | | | | | | | | |
| | | HHHH | | | | | | | | | | | |
| H38 | Chymo | MASMTGGQQMGRDPNSA | SEQ ID NO: | 0 | / | 1 | Yes | 2.435 | / | / | | | / |
| | | HVQLVESGGGLVQAGGSL | 136 | | | | | | | | | | |
| | | RLSCAASELTFSNYAMGW | | | | | | | | | | | |
| | | FRRAPGKERGFVAAISYSG | | | | | | | | | | | |
| | | GSTDYADSVKGRFTISRD | | | | | | | | | | | |
| | | NAKKTVYLQMNSLKPED | | | | | | | | | | | |
| | | TAVYYCAAAYMGWGTA | | | | | | | | | | | |
| | | RSAYEYWGQGTQVTVSSE | | | | | | | | | | | |
| | | PKTPKGGCGGGAAALEH | | | | | | | | | | | |
| | | HHHHH | | | | | | | | | | | |
| H39 | Chymo | MASMTGGQQMGRDPNSA | SEQ ID NO: | / | / | 1 | Yes | 5.196 | / | / | | | EDC |
| | | QVQLVESGGGLVQAGVSL | 137 | | | | | | | | | | |
| | | RLSCAASERTFSSYIMGWF | | | | | | | | | | | |
| | | RQAPGKEREFIAAISWSGG | | | | | | | | | | | |
| | | NTDYAGSVQGRFTISRDN | | | | | | | | | | | |
| | | AQNTVYLQMNSLEPEDTA | | | | | | | | | | | |
| | | VYYCAADATHSWSYGSR | | | | | | | | | | | |
| | | WYDRNYNYWGQGTQVT | | | | | | | | | | | |
| | | VSSEPKTPKGGCGGGAAA | | | | | | | | | | | |
| | | LEHHHHHH | | | | | | | | | | | |
| H40 | Chymo | MASMTGGQQMGRDPNSA | SEQ ID NO: | / | / | 1 | Yes | 3.156 | / | / | | | DSS |
| | | EVQLVESGGGLVQAGASL | 138 | | | | | | | | | | |
| | | RLSCAASGGTFSSYIMGW | | | | | | | | | | | |
| | | FRQAPGKEREFVAAISWS | | | | | | | | | | | |
| | | GRSTHYADSVKGRFAISR | | | | | | | | | | | |
| | | DNDRVYLQMNSLKPEDT | | | | | | | | | | | |
| | | AVYSCAADPNYTWRDDR | | | | | | | | | | | |
| | | YYREEGYTYWGQGTQVT | | | | | | | | | | | |
| | | VSSEPKTPKGGCGGGAAA | | | | | | | | | | | |
| | | LEHHHHHH | | | | | | | | | | | |
| H41 | Chymo | MASMTG | SEQ ID NO: | 1 | / | 1 | No | / | / | / | | | / |
| | | GQQMGR | [symbol] | | | | | | | | | | |
| | | DPNSAQV | | | | | | | | | | | |
| | | QLVESGG | | | | | | | | | | | |
| | | GLVQASS | | | | | | | | | | | |
| | | SLRLSCA | | | | | | | | | | | |
| | | ASGLTFS | | | | | | | | | | | |
| | | NYAMGW | | | | | | | | | | | |
| | | FRQAPGK | | | | | | | | | | | |
| | | EREFVVAI | | | | | | | | | | | |
| | | SRGGNTY | | | | | | | | | | | |
| H42 | Chymo | MASMTGGQ | SEQ ID NO: | 0 | / | 1 | Yes | 4.922 | / | / | | | DSS |
| | | QMGRDPNSA | 140 | | | | | | | | | | |
| | | HVQLVESGG | | | | | | | | | | | |
| | | GLVQAGGSL | | | | | | | | | | | |
| | | RLSCAASGL | | | | | | | | | | | |
| | | TFSNYAMG | | | | | | | | | | | |
| | | WFRQAPGKE | | | | | | | | | | | |
| | | REFVVAISW | | | | | | | | | | | |
| | | SGANTYYSD | | | | | | | | | | | |
| | | SVKGRFTAS | | | | | | | | | | | |
| | | RDNAKKTVY | | | | | | | | | | | |
| H43 | Chymo | MASMTGGQQ | SEQ ID NO: | 2 | 0 | 1 | Yes | No | / | / | | | / |
| | | MGRDPNSAH | 141 | | | | | binding | | | | | |
| | | VQLVESGGG | | | | | | | | | | | |
| | | LVQAGGSLR | | | | | | | | | | | |
| | | LSCAASGLTF | | | | | | | | | | | |
| | | SNYALGWFR | | | | | | | | | | | |
| | | RAPGKERDF | | | | | | | | | | | |
| | | VAAISYSGGS | | | | | | | | | | | |
| | | TDYADSVKG | | | | | | | | | | | |
| | | RFTISRDNAK | | | | | | | | | | | |
| | | NTVYLQMNS | | | | | | | | | | | |
| H44 | Chymo | MASMTGGQ | SEQ ID NO: | 2 | 1 | 1 | Yes | 5.622 | / | / | | | / |
| | | QMGRDPNS | 142 | | | | | | | | | | |
| | | AEVQLVESG | | | | | | | | | | | |
| | | GGLAQAGGS | | | | | | | | | | | |
| | | LRLSCAASG | | | | | | | | | | | |
| | | GTFSNSCMG | | | | | | | | | | | |
| | | WFRQAPGM | | | | | | | | | | | |
| | | EREFVAIIRS | | | | | | | | | | | |
| | | TGHTTYADS | | | | | | | | | | | |
| | | VEGRFTVSR | | | | | | | | | | | |
| | | DIAKNTVYL | | | | | | | | | | | |
| | | | | | | | | | | | | | CX |
| | | | | | | | | | Cross- | CX | CX | CX | Model |
| | | | | | | | | | linked | residue | residue | Model | Epi- |
| | | | | | | | | ID | Peptides | on Nbs | on HSA | Folder | tope |
| | | | | | | | | H1 | SLHTLFGDKL | H1 (98) | HSA(97) | Seq_ | E1 |
| | | | | | | | | | CTVATLR | | | 16529 | |
| | | | | | | | | | (SEQ ID NO: | | | | |
| | | | | | | | | | 2588)(9)- | | | | |
| | | | | | | | | | DNAKNTVYL | | | | |
| | | | | | | | | | QMNR (SEQ | | | | |
| | | | | | | | | | ID NO: | | | | |
| | | | | | | | | | 2589)(4) | | | | |
| | | | | | | | | | DNAKNTVYL | H1 (98) | HSA(249) | | |
| | | | | | | | | | QMNR (SEQ | | | | |
| | | | | | | | | | ID NO: | | | | |
| | | | | | | | | | 2589)(4)- | | | | |
| | | | | | | | | | FPKAEFAEVS | | | | |
| | | | | | | | | | K (SEQ ID NO: | | | | |
| | | | | | | | | | 2590)(3) | | | | |
| | | | | | | | | | ANYNDDSVK | H1 (86) | HSA(161) | | |
| | | | | | | | | | GR (SEQ ID | | | | |
| | | | | | | | | | NO: 2591)(9)- | | | | |
| | | | | | | | | | KYLYEIAR | | | | |
| | | | | | | | | | (SEQ ID NO: | | | | |
| | | | | | | | | | 2592)(1) | | | | |
| | | | | | | | | H2 | SVSQYDYWG | H2 (127) | HSA(375) | Seq_ | E2 |
| | | | | | | | | | QGTQVTVSE | | | 8598 | |
| | | | | | | | | | PKTPK (SEQ | | | | |
| | | | | | | | | | ID NO: | | | | |
| | | | | | | | | | 2593)(20)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | DNAKNAAYL | H2 (77) | HSA(438) | | |
| | | | | | | | | | QMSNLKPED | | | | |
| | | | | | | | | | TAVYYCATG | | | | |
| | | | | | | | | | GGLGYYR | | | | |
| | | | | | | | | | (SEQ ID NO: | | | | |
| | | | | | | | | | 2595)(4)- | | | | |
| | | | | | | | | | KVPQVSTPTL | | | | |
| | | | | | | | | | VEVSR (SEQ | | | | |
| | | | | | | | | | ID NO: | | | | |
| | | | | | | | | | 2596)(1) | | | | |
| | | | | | | | | H3 | YTFQYDYWG | HS (134) | HSA(402) | Seq_ | E2 |
| | | | | | | | | | QGTQVTVSE | | | 14034 | |
| | | | | | | | | | PK (SEQ ID | | | | |
| | | | | | | | | | NO: 2597)(6)- | | | | |
| | | | | | | | | | VFDEFKPLVE | | | | |
| | | | | | | | | | EPQNLIK | | | | |
| | | | | | | | | | (SEQ ID NO: | | | | |
| | | | | | | | | | 2598)(6) | | | | |
| | | | | | | | | | YTFQYDYWG | H3 (134) | HSA(565) | | |
| | | | | | | | | | QGTQVTVSE | | | | |
| | | | | | | | | | PK (SEQ ID | | | | |
| | | | | | | | | | NO: 2597)(6)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | DPENLYFQG | H3 (13) | HSA(383) | | |
| | | | | | | | | | AQVQLVESG | | | | |
| | | | | | | | | | GGLVQAGGS | | | | |
| | | | | | | | | | LR (SEQ ID | | | | |
| | | | | | | | | | NO: 2600)(1)- | | | | |
| | | | | | | | | | TYETTLEKCC | | | | |
| | | | | | | | | | AAADPHECY | | | | |
| | | | | | | | | | AK (SEQ ID | | | | |
| | | | | | | | | | NO: 2601)(8) | | | | |
| | | | | | | | | H4 | VFDEFKPLVE | H4 (82) | HSA(402) | Seq_ | E2 |
| | | | | | | | | | EPQNLIK | | | 29830 | |
| | | | | | | | | | (SEQ ID NO: | | | | |
| | | | | | | | | | 2598)(6)- | | | | |
| | | | | | | | | | SGSSTYYADS | | | | |
| | | | | | | | | | VKGR (SEQ | | | | |
| | | | | | | | | | ID NO: | | | | |
| | | | | | | | | | 2602)(12) | | | | |
| | | | | | | | | | CCAAADPHE | H4 (82) | HSA(396) | | |
| | | | | | | | | | CYAKVFDEF | | | | |
| | | | | | | | | | KPLVEEPQNL | | | | |
| | | | | | | | | | IK (SEQ ID | | | | |
| | | | | | | | | | NO: 2628)(13)- | | | | |
| | | | | | | | | | SGSSTYYADS | | | | |
| | | | | | | | | | VKGR (SEQ | | | | |
| | | | | | | | | | ID NO: | | | | |
| | | | | | | | | | 2602)(12) | | | | |
| | | | | | | | | | VFDEFKPLVE | H4 (79) | HSA(402) | | |
| | | | | | | | | | EPQNLIK | | | | |
| | | | | | | | | | (SEQ ID NO: | | | | |
| | | | | | | | | | 2598)(6)- | | | | |
| | | | | | | | | | SGSSTYYADS | | | | |
| | | | | | | | | | VK (SEQ ID | | | | |
| | | | | | | | | | NO: 2603)(9) | | | | |
| | | | | | | | | | VFDEFKPLVE | H4 (82) | HSA(406) | | |
| | | | | | | | | | EPQNLIK | | | | |
| | | | | | | | | | (SEQ ID NO: | | | | |
| | | | | | | | | | 2598)(10)- | | | | |
| | | | | | | | | | SGSSTYYADS | | | | |
| | | | | | | | | | VKGR (SEQ | | | | |
| | | | | | | | | | ID NO: | | | | |
| | | | | | | | | | 2602)(12) | | | | |
| | | | | | | | | H5 | / | / | / | / | / |
| | | | | | | | | H6 | KMSTVATEA | H6 (115) | HSA(341) | Seq_ | E2 |
| | | | | | | | | | TMYAYWGH | | | 35308 | |
| | | | | | | | | | GTQVTVSSEP | | | | |
| | | | | | | | | | K (SEQ ID NO: | | | | |
| | | | | | | | | | 2604)(1)- | | | | |
| | | | | | | | | | DVCKNYAEA | | | | |
| | | | | | | | | | K (SEQ ID NO: | | | | |
| | | | | | | | | | 2605)(4) | | | | |
| | | | | | | | | H7 | DNAKNTVYL | H7 (93) | HSA(28) | Seq_ | E3 |
| | | | | | | | | | QMNSLKPED | | | 45799 | |
| | | | | | | | | | TAVYYCGAA | | | | |
| | | | | | | | | | DPMGLGYGL | | | | |
| | | | | | | | | | GPRPVDR | | | | |
| | | | | | | | | | (SEQ ID NO: | | | | |
| | | | | | | | | | 2606)(4)- | | | | |
| | | | | | | | | | RDAHKSEVA | | | | |
| | | | | | | | | | HR (SEQ ID | | | | |
| | | | | | | | | | NO: 2607)(5) | | | | |
| | | | | | | | | | EREFVSGISW | H7 (82) | HSA(300) | | |
| | | | | | | | | | SGSSIDYVDS | | | | |
| | | | | | | | | | VKGR (SEQ | | | | |
| | | | | | | | | | ID NO: | | | | |
| | | | | | | | | | 2608)(22)- | | | | |
| | | | | | | | | | LKECCEK | | | | |
| | | | | | | | | | (SEQ ID NO: | | | | |
| | | | | | | | | | 2609)(2) | | | | |
| | | | | | | | | H8 | / | / | / | / | / |
| | | | | | | | | H9 | / | / | / | / | / |
| | | | | | | | | H10 | / | / | / | / | / |
| | | | | | | | | H11 | EFVAYIHWS | H11 (82) | HSA(565) | Seq_ | E4 |
| | | | | | | | | | GSSTSYADSV | | | 20104 | |
| | | | | | | | | | KGR (SEQ ID | | | | |
| | | | | | | | | | NO: 2610)(20)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | DNTKNTMYL | H11 (93) | HSA(581) | | |
| | | | | | | | | | QMNSLKPED | | | | |
| | | | | | | | | | TAVYYCTAD | | | | |
| | | | | | | | | | QYASTLLR | | | | |
| | | | | | | | | | (SEQ ID NO: | | | | |
| | | | | | | | | | 2611)(4)- | | | | |
| | | | | | | | | | AVMDDFAAF | | | | |
| | | | | | | | | | VEKCCK | | | | |
| | | | | | | | | | (SEQ ID NO: | | | | |
| | | | | | | | | | 2612)(12) | | | | |
| | | | | | | | | | DNTKNTMYL | H11 (93) | HSA(584) | | |
| | | | | | | | | | QMNSLKPED | | | | |
| | | | | | | | | | TAVYYCTAD | | | | |
| | | | | | | | | | QYASTLLR | | | | |
| | | | | | | | | | (SEQ ID NO: | | | | |
| | | | | | | | | | 2611)(4)- | | | | |
| | | | | | | | | | CCKADDKET | | | | |
| | | | | | | | | | CFAEEGK | | | | |
| | | | | | | | | | (SEQ ID NO: | | | | |
| | | | | | | | | | 2613)(3) | | | | |
| | | | | | | | | H12 | / | / | / | / | / |
| | | | | | | | | H13 | / | / | / | / | / |
| | | | | | | | | H14 | / | / | / | / | / |
| | | | | | | | | H15 | / | / | / | / | / |
| | | | | | | | | H16 | / | / | / | / | / |
| | | | | | | | | H17 | / | / | / | / | / |
| | | | | | | | | H18 | LSCTASGPKD | H18 (45) | HSA(375) | Seq_ | E2 |
| | | | | | | | | | TPYTMGWFR | | | 45285 | |
| | | | | | | | | | (SEQ ID NO: | | | | |
| | | | | | | | | | 2614)(9)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | LSCTASGPKD | H18(45) | HSA(438) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | KVPQVSTPTL | | | | |
| | | | | | | | | | VEVSR (SEQ | | | | |
| | | | | | | | | | ID NO: | | | | |
| | | | | | | | | | 2596)(1) | | | | |
| | | | | | | | | | LSCTASGPKD | H18(45) | HSA(499) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | LSCTASGPKD | H18*45) | HSA(565) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | LSCTASGPKD | H18(45) | HSA(516) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | RPCFSALEVD | | | | |
| | | | | | | | | | ETYVPK (SEQ | | | | |
| | | | | | | | | | ID NO: | | | | |
| | | | | | | | | | 2616)(8) | | | | |
| | | | | | | | | | DPNSAEVQL | H18(13) | HSA(375) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(1)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | DPNSAEVQL | H18(18) | HSA(499) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(6)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | DPNSAEVQL | H18(13) | HSA(499) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(1)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | LSCTASGPKD | H18(45) | HSA(407) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | VFDEFKPLVE | | | | |
| | | | | | | | | | EPQNLIK | | | | |
| | | | | | | | | | (SEQ ID NO: | | | | |
| | | | | | | | | | 2598)(11) | | | | |
| | | | | | | | | | LSCTASGPKD | H18(45) | HSA(503) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | CCTESLVNR | | | | |
| | | | | | | | | | (SEQ ID NO: | | | | |
| | | | | | | | | | 2618)(4) | | | | |
| | | | | | | | | H19 | / | / | / | / | / |
| | | | | | | | | H20 | LSCTASGPKD | H20(45) | HSA(375) | Seq_ | E2 |
| | | | | | | | | | TPYTMGWFR | | | 45284 | |
| | | | | | | | | | (SEQ ID NO: | | | | |
| | | | | | | | | | 2614)(9)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | LSCTASGPKD | H20(45) | HSA(499) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | LSCTASGPKD | H20(45) | HSA(438) | | |
| | | | | | | | | | TPYTMGWFR | | | | |
| | | | | | | | | | (SEQ ID NO: | | | | |
| | | | | | | | | | 2614)(9)- | | | | |
| | | | | | | | | | KVPQVSTPTL | | | | |
| | | | | | | | | | VEVSR (SEQ | | | | |
| | | | | | | | | | ID NO: | | | | |
| | | | | | | | | | 2596)(1) | | | | |
| | | | | | | | | | LSCTASGPKD | H20 [symbol] | HSA [symbol] | | |
| | | | | | | | | | TPYTMGWFR | | | | |
| | | | | | | | | | (SEQ ID NO: | | | | |
| | | | | | | | | | 2614)(9)- | | | | |
| | | | | | | | | | RPCFSALEVD | | | | |
| | | | | | | | | | ETYVPK (SEQ | | | | |
| | | | | | | | | | ID NO: | | | | |
| | | | | | | | | | 2616)(8) | | | | |
| | | | | | | | | | DPNSAEVQL | H20(13) | HSA(375) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(1)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | DPNSAEVQL | H20(18) | HSA(499) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(6)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | DPNSAEVQL | H20(13) | HSA(499) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2617)(1)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | H21 | WSGVSTYYA | H21(82) | HSA(300) | Seq_ | E5 |
| | | | | | | | | | DSVKGR | | | 6523 | |
| | | | | | | | | | (SEQ ID NO: | | | | |
| | | | | | | | | | 2619)(13)- | | | | |
| | | | | | | | | | LKECCEK | | | | |
| | | | | | | | | | (SEQ ID NO: | | | | |
| | | | | | | | | | 2609)(2) | | | | |
| | | | | | | | | | DGAKNTLYL | H21(93) | HSA(198) | | |
| | | | | | | | | | QMNSLKPED | | | | |
| | | | | | | | | | TAVYYCAAK | | | | |
| | | | | | | | | | (SEQ ID NO: | | | | |
| | | | | | | | | | 2620)(4)- | | | | |
| | | | | | | | | | AAFTECCQA | | | | |
| | | | | | | | | | ADKAACLLP | | | | |
| | | | | | | | | | K (SEQ ID NO: | | | | |
| | | | | | | | | | 2621)(12) | | | | |
| | | | | | | | | H22 | TPKGGCGGA | H22(147) | HSA(236) | Seq_ | E3 |
| | | | | | | | | | AALEHHHHH | | | 2558 | |
| | | | | | | | | | H (SEQ ID NO: | | | | |
| | | | | | | | | | 2622)(3)- | | | | |
| | | | | | | | | | AFKAWAVA | | | | |
| | | | | | | | | | R (SEQ ID NO: | | | | |
| | | | | | | | | | 2623)(3) | | | | |
| | | | | | | | | H23 | / | / | / | / | / |
| | | | | | | | | H24 | / | / | / | / | / |
| | | | | | | | | H25 | NYDFWGQGT | H25(144) | HSA(161) | Seq_ | E3 |
| | | | | | | | | | QVTVSSEPKT | | | 4162 | |
| | | | | | | | | | PK (SEQ ID | | | | |
| | | | | | | | | | NO: 2623)(18)- | | | | |
| | | | | | | | | | KYLYEIAR | | | | |
| | | | | | | | | | (SEQ ID NO: | | | | |
| | | | | | | | | | 2592)(1) | | | | |
| | | | | | | | | H26 | LAKTYETTLE | H26(87) | HSA(375) | Seq_ | E2 |
| | | | | | | | | | K (SEQ ID NO: | | | 18634 | |
| | | | | | | | | | 2594)(3)- | | | | |
| | | | | | | | | | ISKDNAK | | | | |
| | | | | | | | | | (SEQ ID NO: | | | | |
| | | | | | | | | | 2625)(3) | | | | |
| | | | | | | | | | VFDEFKPLVE | H26(87) | HSA(402) | | |
| | | | | | | | | | EPQNLIK | | | | |
| | | | | | | | | | (SEQ ID NO: | | | | |
| | | | | | | | | | 2598)(6)- | | | | |
| | | | | | | | | | ISKDNAK | | | | |
| | | | | | | | | | (SEQ ID NO: | | | | |
| | | | | | | | | | 2625)(3) | | | | |
| | | | | | | | | | DNAKNTVLL | H26(91) | HSA(402) | | |
| | | | | | | | | | QMNSLKPED | | | | |
| | | | | | | | | | TAVYYCAAR | | | | |
| | | | | | | | | | (SEQ ID NO: | | | | |
| | | | | | | | | | 2626)(4)- | | | | |
| | | | | | | | | | VFDEFKPLVE | | | | |
| | | | | | | | | | EPQNLIK | | | | |
| | | | | | | | | | (SEQ ID NO: | | | | |
| | | | | | | | | | 2598)(6) | | | | |
| | | | | | | | | | NTVLLQMNS | H26(102) | HSA(375) | | |
| | | | | | | | | | LKPEDTAVY | | | | |
| | | | | | | | | | YCAAR (SEQ | | | | |
| | | | | | | | | | ID NO: | | | | |
| | | | | | | | | | 2627)(11)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | | TYETTLEKCC | H26(87) | HSA(383) | | |
| | | | | | | | | | AAADPHECY | | | | |
| | | | | | | | | | AK (SEQ ID | | | | |
| | | | | | | | | | NO: 2601)(8)- | | | | |
| | | | | | | | | | ISKDNAK | | | | |
| | | | | | | | | | (SEQ ID NO: | | | | |
| | | | | | | | | | 2625)(3) | | | | |
| | | | | | | | | | CCAAADPHE | H26(87) | HSA(396) | | |
| | | | | | | | | | CYAKVFDEF | | | | |
| | | | | | | | | | KPLVEEPQNL | | | | |
| | | | | | | | | | IK (SEQ ID | | | | |
| | | | | | | | | | NO: 2628)(13)- | | | | |
| | | | | | | | | | ISKDNAK | | | | |
| | | | | | | | | | (SEQ ID NO: | | | | |
| | | | | | | | | | 2625)(3) | | | | |
| | | | | | | | | | CCAAADPHE | H26(81) | HSA(396) | | |
| | | | | | | | | | CYAKVFDEF | | | | |
| | | | | | | | | | KPLVEEPQNL | | | | |
| | | | | | | | | | IK (SEQ ID | | | | |
| | | | | | | | | | NO: 2628)(13)- | | | | |
| | | | | | | | | | DNAKNTVLL | | | | |
| | | | | | | | | | QMNSLKPED | | | | |
| | | | | | | | | | TAVYYCAAR | | | | |
| | | | | | | | | | (SEQ ID NO: | | | | |
| | | | | | | | | | 2626)(4) | | | | |
| | | | | | | | | | EFVASISWTY | H26(80) | HSA(499) | | |
| | | | | | | | | | GYTNYANSV | | | | |
| | | | | | | | | | KGR (SEQ ID | | | | |
| | | | | | | | | | NO: 2629)(20)- | | | | |
| | | | | | | | | | VTKCCTESL | | | | |
| | | | | | | | | | VNR (SEQ ID | | | | |
| | | | | | | | | | NO: 2615)(3) | | | | |
| | | | | | | | | | RGEDPEYDY | H26(120) | HSA(565) | | |
| | | | | | | | | | WGQGTQVT | | | | |
| | | | | | | | | | VSSEPK (SEQ | | | | |
| | | | | | | | | | ID NO: | | | | |
| | | | | | | | | | 2630)(6)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | RGEDPEYDY | H26(122) | HSA(565) | | |
| | | | | | | | | | WGQGTQVT | | | | |
| | | | | | | | | | VSSEPK (SEQ | | | | |
| | | | | | | | | | ID NO: | | | | |
| | | | | | | | | | 2630)(8)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | DPNSAQVQL | H26(13) | HSA(565) | | |
| | | | | | | | | | VESGGGLVQ | | | | |
| | | | | | | | | | AGGSLR (SEQ | | | | |
| | | | | | | | | | ID NO: | | | | |
| | | | | | | | | | 2631)(1)- | | | | |
| | | | | | | | | | ATKEQLK | | | | |
| | | | | | | | | | (SEQ ID NO: | | | | |
| | | | | | | | | | 2599)(3) | | | | |
| | | | | | | | | H27 | EFVAAITQNG | H27(82) | HSA(28) | Seq_ | E3 |
| | | | | | | | | | GTTYYADSV | | | 10156 | |
| | | | | | | | | | KGR (SEQ ID | | | | |
| | | | | | | | | | NO: 2632)(20)- | | | | |
| | | | | | | | | | DAHKSEVAH | | | | |
| | | | | | | | | | R (SEQ ID NO: | | | | |
| | | | | | | | | | 2633)(4) | | | | |
| | | | | | | | | | EFVAAITQNG | H27(82) | HSA(305) | | |
| | | | | | | | | | GTTYYADSV | | | | |
| | | | | | | | | | KGR (SEQ ID | | | | |
| | | | | | | | | | NO: 2632)(20)- | | | | |
| | | | | | | | | | ECCEKPLLEK | | | | |
| | | | | | | | | | (SEQ ID NO: | | | | |
| | | | | | | | | | 2634)(5) | | | | |
| | | | | | | | | | EFVAAITQNG | H27(82) | HSA(36) | | |
| | | | | | | | | | GTTYYADSV | | | | |
| | | | | | | | | | KGR (SEQ ID | | | | |
| | | | | | | | | | NO: 2632)(20)- | | | | |
| | | | | | | | | | FKDLGEENF | | | | |
| | | | | | | | | | K (SEQ ID NO: | | | | |
| | | | | | | | | | 2635)(2) | | | | |
| | | | | | | | | | FKDLGEENF | H27(60) | HSA(36 [symbol] | | |
| | | | | | | | | | K (SEQ ID NO: | | | | |
| | | | | | | | | | 2635)(2)- | | | | |
| | | | | | | | | | EAPGKGR | | | | |
| | | | | | | | | | (SEQ ID NO: | | | | |
| | | | | | | | | | 2636)(5) | | | | |
| | | | | | | | | H28 | NTVYLEMNN | N28(103) | HSA(565) | Seq_ | E2 |
| | | | | | | | | | LKPEDTAVY | | | 14266 | |
| | | | | | | | | | SCGAGVSDY | | | | |
| | | | | | | | | | GCYR (SEQ | | | | |
| | | | | | | | | | ID NO: | | | | |
| | | | | | | | | | 2637)(11)- | | | | |
| | | | | | | | | | ATKEQLK | | | | |
| | | | | | | | | | (SEQ ID NO: | | | | |
| | | | | | | | | | 2599)(3) | | | | |
| | | | | | | | | | TSGYNYWGQ | N28(143) | HSA(375) | | |
| | | | | | | | | | GTQ VTVS SEP | | | | |
| | | | | | | | | | KTPK (SEQ ID | | | | |
| | | | | | | | | | NO: 2638)(20)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | H29 | / | / | / | / | / |
| | | | | | | | | H30 | / | / | / | / | / |
| | | | | | | | | H31 | / | / | / | / | / |
| | | | | | | | | H32 | / | / | / | / | / |
| | | | | | | | | H33 | LAKTYETTLE | H33(82) | HSA(375) | Seq_ | E2 |
| | | | | | | | | | K (SEQ ID NO: | | | 28093 | |
| | | | | | | | | | 2594)(3)- | | | | |
| | | | | | | | | | DSVKGR | | | | |
| | | | | | | | | | (SEQ ID NO: | | | | |
| | | | | | | | | | 2639)(4) | | | | |
| | | | | | | | | | CCAAADPHE | H33(93) | HSA(396) | | |
| | | | | | | | | | CYAKVFDEF | | | | |
| | | | | | | | | | KPLVEEPQNL | | | | |
| | | | | | | | | | IK (SEQ ID | | | | |
| | | | | | | | | | NO: 2628)(13)- | | | | |
| | | | | | | | | | DNAKNTVYL | | | | |
| | | | | | | | | | QMNSLK | | | | |
| | | | | | | | | | (SEQ ID NO: | | | | |
| | | | | | | | | | 2640)(4) | | | | |
| | | | | | | | | | DNAKNTVYL | H33(93) | HSA(375) | | |
| | | | | | | | | | QMNSLK | | | | |
| | | | | | | | | | (SEQ ID NO: | | | | |
| | | | | | | | | | 2640)(4)- | | | | |
| | | | | | | | | | LAKTYETTLE | | | | |
| | | | | | | | | | K (SEQ ID NO: | | | | |
| | | | | | | | | | 2594)(3) | | | | |
| | | | | | | | | H34 | PLVEEPQNLI | H34(82) | HSA(413) | Seq_ | E2 |
| | | | | | | | | | KQNCELFEQ | | | 9366 | |
| | | | | | | | | | LGEYK(11)- | | | | |
| | | | | | | | | | DSVKGR | | | | |
| | | | | | | | | | (SEQ ID NO: | | | | |
| | | | | | | | | | 2639)(4) | | | | |
| | | | | | | | | | CCAAADPHE | H34(93) | HSA(396) | | |
| | | | | | | | | | CYAKVFDEF | | | | |
| | | | | | | | | | KPLVEEPQNL | | | | |
| | | | | | | | | | IK (SEQ ID | | | | |
| | | | | | | | | | NO: 2628)(13)- | | | | |
| | | | | | | | | | DNAKNTVYL | | | | |
| | | | | | | | | | QMNSLK | | | | |
| | | | | | | | | | (SEQ ID NO: | | | | |
| | | | | | | | | | 2640)(4) | | | | |
| | | | | | | | | | VFDEFKPLVE | H34(72) | HAS(402) | | |
| | | | | | | | | | EPQNLIK | | | | |
| | | | | | | | | | (SEQ ID NO: | | | | |
| | | | | | | | | | 2598)(6)- | | | | |
| | | | | | | | | | HDYNTYYR | | | | |
| | | | | | | | | | (SEQ ID NO: | | | | |
| | | | | | | | | | 2641)(2) | | | | |
| | | | | | | | | H35 | / | / | / | / | / |
| | | | | | | | | H36 | / | / | / | / | / |
| | | | | | | | | H37 | / | / | / | / | / |
| | | | | | | | | H38 | / | / | / | / | / |
| | | | | | | | | H39 | SHCIAEVEND | H39(2) | HSA(325) | Seq_ | E2 |
| | | | | | | | | | EMPADLPSL | | | 43495 | |
| | | | | | | | | | AADFVESK | | | | |
| | | | | | | | | | (SEQ ID NO: | | | | |
| | | | | | | | | | 2642)(15)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | RPCFSALEVD | H39(2) | HSA(516) | | |
| | | | | | | | | | ETYVPK (SEQ | | | | |
| | | | | | | | | | ID NO: | | | | |
| | | | | | | | | | 2616)(8)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | SHCIAEVEND | H39(2) | HSA(332) | | |
| | | | | | | | | | EMPADLPSL | | | | |
| | | | | | | | | | AADFVESK | | | | |
| | | | | | | | | | (SEQ ID NO: | | | | |
| | | | | | | | | | 2642)(22)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | | SHCIAEVEND | H39(2) | HSA(321) | | |
| | | | | | | | | | EMPADLPSL | | | | |
| | | | | | | | | | AADFVESK | | | | |
| | | | | | | | | | (SEQ ID NO: | | | | |
| | | | | | | | | | 2642)(11)- | | | | |
| | | | | | | | | | ASMTGGQQ | | | | |
| | | | | | | | | | MGR (SEQ ID | | | | |
| | | | | | | | | | NO: 2546)(1) | | | | |
| | | | | | | | | H40 | LAKTYETTLE | H40(82) | HSA(375) | Seq_ | E2 |
| | | | | | | | | | K (SEQ ID NO: | | | 45710 | |
| | | | | | | | | | 2594)(3)- | | | | |
| | | | | | | | | | STHYADSVK | | | | |
| | | | | | | | | | GR (SEQ ID | | | | |
| | | | | | | | | | NO: 2643)(9) | | | | |
| | | | | | | | | H41 | / | / | / | / | / |
| | | | | | | | | H42 | KTVYLQMNS | H42(94) | HSA(468) | Seq_ | E2 |
| | | | | | | | | | LKPEDTAVY | | | 44732 | |
| | | | | | | | | | YCAADYR | | | | |
| | | | | | | | | | (SEQ ID NO: | | | | |
| | | | | | | | | | 2644)(1)- | | | | |
| | | | | | | | | | HPEAKR | | | | |
| | | | | | | | | H43 | / | / | / | / | / |
| | | | | | | | | H44 | / | / | / | / | / |

[symbol]

indicates data missing or illegible when filed

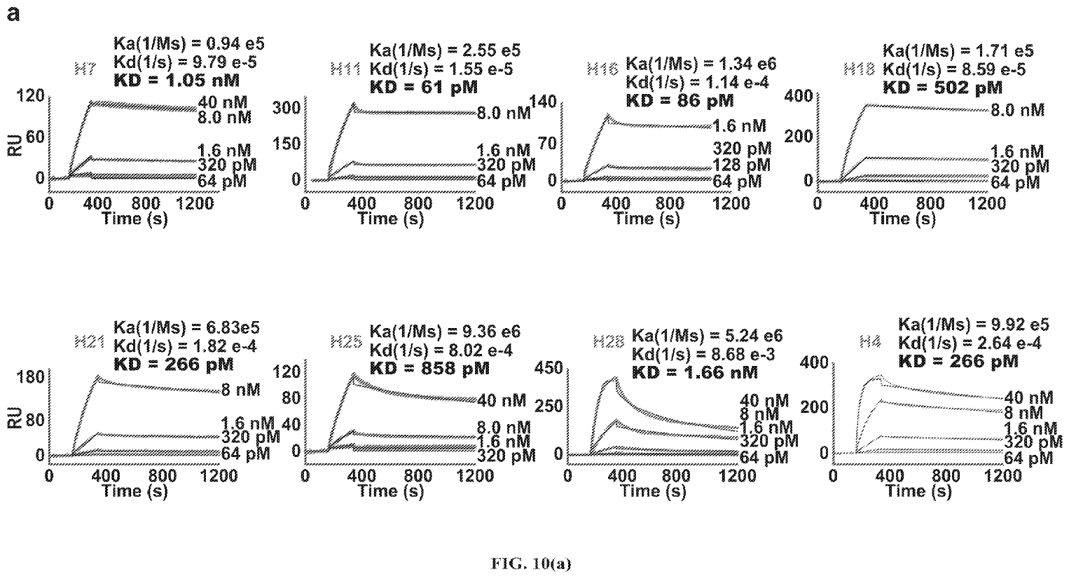

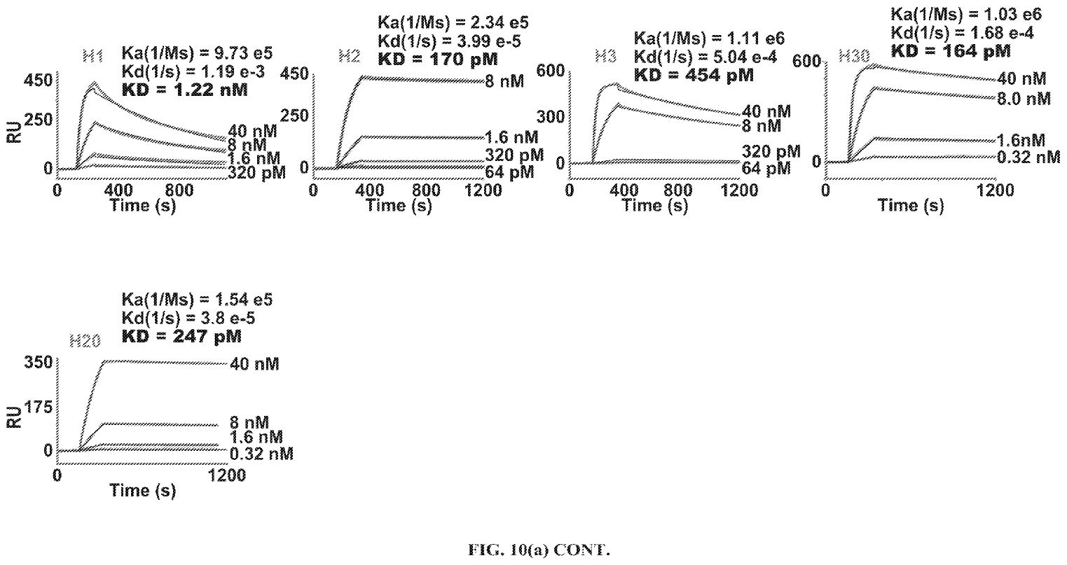

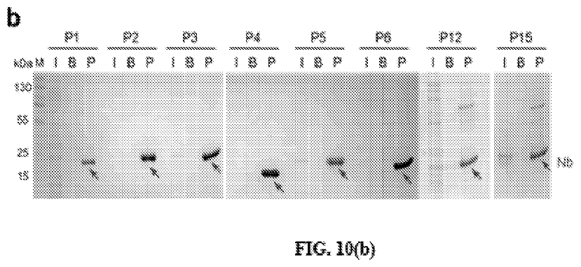

TABLE 3
Summary of PDZ Nbs and their biophysical and physiochemical properties.

| | | | | | Bind by | WT | Mutant | |
|---|---|---|---|---|---|---|---|---|
| | | | | | beads- | ELISA | ELISA | |
| | | | | | binding | affinity | affinity | Affinity |
| | | | SEQ ID | | assay | (LogIC50 | (LogIC50 | fold |
| ID | Enzyme | Protein Sequences | NO | Soluble | (FIG. 10b) | (oD450nm)) | (oD450nm)) | change |
| P1 | Trypsin/ | MASMTG | SEQ ID | Yes | Yes | / | / | / |
| | Chymo | GQQMGR | NO: 143 | | | | | |
| | | NSADVQL | | | | | | |
| | | VESGGGL | | | | | | |
| | | VQPGGSL | | | | | | |
| | | RLSCAAS | | | | | | |
| | | GFTLDDY | | | | | | |
| | | AIGWFRQ | | | | | | |
| | | APGKERE | | | | | | |
| | | GVSCISSH | | | | | | |
| | | GSTYYAD | | | | | | |
| | | SVKGRFTI | | | | | | |
| | | SRDNVKN | | | | | | |
| | | TLYLQMN | | | | | | |
| | | SLKPEDT | | | | | | |
| | | ALYYCAA | | | | | | |
| | | SYYSDYE | | | | | | |
| | | VAVCRSD | | | | | | |
| | | ALDAWG | | | | | | |
| | | GQTQVTV | | | | | | |
| | | SEPKTPK | | | | | | |
| | | GGCGGGL | | | | | | |
| | | EHHHHHH | | | | | | |
| P2 | Trypsin/ | MASMTG | SEQ ID | Yes | Yes | 5.437 | 4.354 | 12.106 |
| | Chymo | GQQMGR | NO: 144 | | | | | |
| | | NSADVQL | | | | | | |
| | | VESGGGL | | | | | | |
| | | VQAGGSL | | | | | | |
| | | RLSCAAS | | | | | | |
| | | GHTFSSY | | | | | | |
| | | TMGWFH | | | | | | |
| | | QAPGKER | | | | | | |
| | | EFVAEISG | | | | | | |
| | | TGGNTGY | | | | | | |
| | | ASDVKGR | | | | | | |
| | | FTISRDNA | | | | | | |
| | | KNTVYLQ | | | | | | |
| | | MNSLKPE | | | | | | |
| | | DTAVYYC | | | | | | |
| | | AAVIGSPT | | | | | | |
| | | DSSDYRS | | | | | | |
| | | SLDYDYW | | | | | | |
| | | GQGTQVT | | | | | | |
| | | VSEPKTP | | | | | | |
| | | KGGCGGG | | | | | | |
| | | LEHHHHH | | | | | | |
| | | H | | | | | | |
| P3 | Trypsin/ | MASMTG | SEQ ID | Yes | Yes | 5.264 | 4.781 | 3.04089 |
| | Chymo | GQQMGR | NO: 145 | | | | | |
| | | NSADVQL | | | | | | |
| | | VESGGGL | | | | | | |
| | | VQAGGSL | | | | | | |
| | | RLSCAAS | | | | | | |
| | | GRTFSSYT | | | | | | |
| | | MGWFHQ | | | | | | |
| | | APGKERE | | | | | | |
| | | FVAEIGGT | | | | | | |
| | | GGNTGYA | | | | | | |
| | | DSVKGRF | | | | | | |
| | | TISRDNA | | | | | | |
| | | KNTVYLQ | | | | | | |
| | | MNSLKPE | | | | | | |
| | | DTAVYYC | | | | | | |
| | | AAVIGSPT | | | | | | |
| | | DSSDYRS | | | | | | |
| | | SLDYDYW | | | | | | |
| | | GQGTQVT | | | | | | |
| | | VSEPKTP | | | | | | |
| | | KGGCGGG | | | | | | |
| | | LEHHHHH | | | | | | |
| | | H | | | | | | |
| P4 | Trypsin/ | MASMTGG | SEQ ID | Yes | Yes | 4.425 | 4.578 | 1 |
| | Chymo | QQMGRNS | NO: 146 | | | | | |
| | | AHVQLVE | | | | | | |
| | | SGGGLVQ | | | | | | |
| | | AGGSLRL | | | | | | |
| | | SCAAAGR | | | | | | |
| | | TSSDYAM | | | | | | |
| | | GWFRQAP | | | | | | |
| | | GKEREFV | | | | | | |
| | | SAINWSGI | | | | | | |
| | | STYYADS | | | | | | |
| | | VKGRFTIS | | | | | | |
| | | RDNAKNT | | | | | | |
| | | VHLQMNS | | | | | | |
| | | LKPEDTA | | | | | | |
| | | VYYCAAE | | | | | | |
| | | KLESLRN | | | | | | |
| | | WHDPLM | | | | | | |
| | | YDYWGQ | | | | | | |
| | | GTQVTVS | | | | | | |
| | | EPKTPKG | | | | | | |
| | | GCGGGLE | | | | | | |
| | | HHHHHH | | | | | | |
| P5 | Trypsin/ | MASMTGG | SEQ ID | Yes | Yes | 4.704 | 0 | 50582.5 |
| | Chymo | QQMGRNS | NO: 147 | | | | | |
| | | ADVQLVES | | | | | | |
| | | GGGLVQA | | | | | | |
| | | GSSLRLSC | | | | | | |
| | | AASGITFR | | | | | | |
| | | WYTMAWF | | | | | | |
| | | RQAPGKER | | | | | | |
| | | DFVATINW | | | | | | |
| | | SGSDTNYA | | | | | | |
| | | DSVKGRFTI | | | | | | |
| | | SRDNAKNT | | | | | | |
| | | VTLQMNSL | | | | | | |
| | | QPEDTAVY | | | | | | |
| | | YCAGVPGT | | | | | | |
| | | SLSGETDPR | | | | | | |
| | | DYDYWGQ | | | | | | |
| | | GTQVTVSE | | | | | | |
| | | PKTPKGGC | | | | | | |
| | | GGGLEHHH | | | | | | |
| | | HHH | | | | | | |
| P6 | Trypsin/ | MASMTG | SEQ ID | Yes | Yes | 5.247 | 4.726 | 3.31895 |
| | Chymo | GQQMGR | NO: 148 | | | | | |
| | | NSAHVQL | | | | | | |
| | | VESGGGL | | | | | | |
| | | VQAGGSL | | | | | | |
| | | RLSCAAS | | | | | | |
| | | GRTFSRY | | | | | | |
| | | RMGWFH | | | | | | |
| | | QAPGKER | | | | | | |
| | | EFVAEISG | | | | | | |
| | | TGGNTGY | | | | | | |
| | | ADSVKGR | | | | | | |
| | | FTMSRDN | | | | | | |
| | | AKNTVYL | | | | | | |
| | | QMNSLKP | | | | | | |
| | | EDTGVYY | | | | | | |
| | | CAAVIGSP | | | | | | |
| | | TDSSDYR | | | | | | |
| | | SSLDYDY | | | | | | |
| | | WGQGTQ | | | | | | |
| | | VTVSEPK | | | | | | |
| | | TKPGGCG | | | | | | |
| | | GGLEHHH | | | | | | |
| | | HHH | | | | | | |
| P7 | Trypsin | MASMTG | SEQ ID | No | / | / | / | / |
| | | GQQMGR | NO: 149 | | | | | |
| | | NSADVQL | | | | | | |
| | | VESGGGL | | | | | | |
| | | VKPGESL | | | | | | |
| | | KLSCVAS | | | | | | |
| | | GETLSSYI | | | | | | |
| | | MGWFRQ | | | | | | |
| | | APGKERE | | | | | | |
| | | FVAAVSW | | | | | | |
| | | SGNQQDY | | | | | | |
| | | ADSVKGR | | | | | | |
| | | FTISRDNA | | | | | | |
| | | KNTVYLQ | | | | | | |
| | | MNSLKPE | | | | | | |
| | | DTAVYYC | | | | | | |
| | | ANGPCTG | | | | | | |
| | | PRAIAEVL | | | | | | |
| | | YESWGQG | | | | | | |
| | | TQVTVSE | | | | | | |
| | | PKTPKGG | | | | | | |
| | | CGGGLEH | | | | | | |
| | | HHHHH | | | | | | |
| P8 | Trypsin | MASMTG | SEQ ID | No | / | / | / | / |
| | | GQQMGR | NO: 150 | | | | | |
| | | NSAEVQL | | | | | | |
| | | VESGGGL | | | | | | |
| | | VQPGGSL | | | | | | |
| | | RLSDKAS | | | | | | |
| | | GFDFEYY | | | | | | |
| | | TIGWFRQ | | | | | | |
| | | APGKERE | | | | | | |
| | | GVSCINR | | | | | | |
| | | GDGATYY | | | | | | |
| | | RDSVKGR | | | | | | |
| | | FTISRDNA | | | | | | |
| | | KKTMYLE | | | | | | |
| | | MNNLKPE | | | | | | |
| | | DTAVYYC | | | | | | |
| | | ATADSGW | | | | | | |
| | | GCYGHRI | | | | | | |
| | | QKNEFDH | | | | | | |
| | | FGQGTQV | | | | | | |
| | | TVSEPKTP | | | | | | |
| | | KGGCGGG | | | | | | |
| | | LEHHHHH | | | | | | |
| | | H | | | | | | |
| P9 | Chymo | MASMTG | SEQ ID | Yes | / | 4.878 | 2.61 | 185.353 |
| | | GQQMGR | NO: 151 | | | | | |
| | | NSADVQL | | | | | | |
| | | VESGGGL | | | | | | |
| | | VQAGGSL | | | | | | |
| | | RLSCVAS | | | | | | |
| | | VASGRTF | | | | | | |
| | | GWYDMG | | | | | | |
| | | WFRQAPG | | | | | | |
| | | KEREFVA | | | | | | |
| | | AISWSGG | | | | | | |
| | | STYYADS | | | | | | |
| | | VKGRSTIS | | | | | | |
| | | RDNAKNT | | | | | | |
| | | VYLQMNS | | | | | | |
| | | LKPEDTA | | | | | | |
| | | VYYCAAR | | | | | | |
| | | GGGTSVD | | | | | | |
| | | SDYDVGE | | | | | | |
| | | FEYDYWG | | | | | | |
| | | QGTQVTV | | | | | | |
| | | SEPKTPK | | | | | | |
| | | GGCGGGL | | | | | | |
| | | EHHHHHH | | | | | | |
| P10 | Chymo | MASMTG | SEQ ID | Yes | / | 5.205 | 3.834 | 23.4963 |
| | | GQQMGR | NO: 152 | | | | | |
| | | NSADVQL | | | | | | |
| | | VESGGGL | | | | | | |
| | | VQAGGSL | | | | | | |
| | | RLSCTAS | | | | | | |
| | | GRTFSTY | | | | | | |
| | | TMAWFR | | | | | | |
| | | QAPGKER | | | | | | |
| | | EFVAAIT | | | | | | |
| | | WSGTYYA | | | | | | |
| | | DSVKGRF | | | | | | |
| | | TISRDNA | | | | | | |
| | | KNTMYLQ | | | | | | |
| | | MNSLKPE | | | | | | |
| | | DTAVYIC | | | | | | |
| | | AAVIGST | | | | | | |
| | | VDSYSPS | | | | | | |
| | | DPLEYDY | | | | | | |
| | | WGQGTQ | | | | | | |
| | | VTVSEPK | | | | | | |
| | | TPKGGCG | | | | | | |
| | | GGLEHHH | | | | | | |
| | | HHH | | | | | | |
| P11 | Trypsin/ | MASMTGG | SEQ ID | Yes | / | 4.068 | 0 | 11695 |
| | Chymo | QQMGRNS | NO: 153 | | | | | |
| | | ADVQLVE | | | | | | |
| | | SGGGLVQ | | | | | | |
| | | AGGSLRLS | | | | | | |
| | | CVASGRT | | | | | | |
| | | FSTYTMG | | | | | | |
| | | WFRQAPG | | | | | | |
| | | KEREFVA | | | | | | |
| | | HIGWSGSS | | | | | | |
| | | TYYADSV | | | | | | |
| | | KGRFTISR | | | | | | |
| | | DNAKNTM | | | | | | |
| | | YLQMNSL | | | | | | |
| | | KPEDTAV | | | | | | |
| | | YYCAVAI | | | | | | |
| | | GSPVDSY | | | | | | |
| | | RHSDPLEY | | | | | | |
| | | DYWGQGT | | | | | | |
| | | QVTVSEP | | | | | | |
| | | KTPKGGC | | | | | | |
| | | GGGLEHH | | | | | | |
| | | HHHH | | | | | | |
| P12 | Chymo | MASMTG | SEQ ID | Yes | Yes | / | / | / |
| | | GQQMGR | NO: 154 | | | | | |
| | | NSAQVQL | | | | | | |
| | | VESGGGL | | | | | | |
| | | VQAGGSL | | | | | | |
| | | RLSCTAS | | | | | | |
| | | GRTFSTY | | | | | | |
| | | TMAWFR | | | | | | |
| | | QAPGKER | | | | | | |
| | | EFVAAIS | | | | | | |
| | | WSGAYY | | | | | | |
| | | AESVKGR | | | | | | |
| | | FTISRDNA | | | | | | |
| | | KNTVYLQ | | | | | | |
| | | MNSLKPE | | | | | | |
| | | DTAVYYC | | | | | | |
| | | AAVIGST | | | | | | |
| | | VDSYSPS | | | | | | |
| | | DPLEYDY | | | | | | |
| | | WARGPRS | | | | | | |
| | | PSEPKTPK | | | | | | |
| | | GGCGGGL | | | | | | |
| | | EHHHHHH | | | | | | |
| P13 | Chymo | MASMTG | SEQ ID | Yes | / | 4.454 | 0.0071 | 27982.3 |
| | | GQQMGR | NO: 155 | | | | | |
| | | NSAQVQL | | | | | | |
| | | VESGGGL | | | | | | |
| | | VQAGGSL | | | | | | |
| | | RLSCAAS | | | | | | |
| | | GRTFSTY | | | | | | |
| | | TMGWFR | | | | | | |
| | | QAPGKER | | | | | | |
| | | EFVAAVT | | | | | | |
| | | WSETLYS | | | | | | |
| | | DSVKGRF | | | | | | |
| | | TISRDNA | | | | | | |
| | | KNTVYLQ | | | | | | |
| | | MNSLKPE | | | | | | |
| | | DTAVYYC | | | | | | |
| | | AAVQGSP | | | | | | |
| | | VDTIVVL | | | | | | |
| | | TTSEEYD | | | | | | |
| | | YWGQGT | | | | | | |
| | | QVTVSSE | | | | | | |
| | | PKTPKGG | | | | | | |
| | | CGGGLEH | | | | | | |
| | | HHHHH | | | | | | |
| P14 | Chymo | MASMTG | SEQ ID | Yes | / | 5.151 | 3.741 | 25.704 |
| | | GQQMGR | NO: 156 | | | | | |
| | | NSAQVQL | | | | | | |
| | | VESGGGL | | | | | | |
| | | VQAGDSL | | | | | | |
| | | RLSCTAS | | | | | | |
| | | GRTFSTY | | | | | | |
| | | TMAWFR | | | | | | |
| | | QAPGKER | | | | | | |
| | | EFVAAIS | | | | | | |
| | | WSGTYYA | | | | | | |
| | | DSVKGRF | | | | | | |
| | | TISRDNA | | | | | | |
| | | KNTVYLQ | | | | | | |
| | | MNSLKPE | | | | | | |
| | | DTAVYYC | | | | | | |
| | | AAVIGST | | | | | | |
| | | VDTYSPS | | | | | | |
| | | DPLEYDA | | | | | | |
| | | WGQGTQ | | | | | | |
| | | VTVSSEP | | | | | | |
| | | KTPKGGS | | | | | | |
| | | GGGLEHH | | | | | | |
| | | HHHH | | | | | | |
| P15 | Chymo | MASMTG | SEQ ID | Yes | Yes | 4.971 | 1.657 | 2060.63 |
| | | GQQMGR | NO: 157 | | | | | |
| | | NSAQVQL | | | | | | |
| | | VESGGGL | | | | | | |
| | | VQAGGSL | | | | | | |
| | | RLSCVAS | | | | | | |
| | | GRPFSSLD | | | | | | |
| | | MGWFRQ | | | | | | |
| | | RPGKERD | | | | | | |
| | | VVATINW | | | | | | |
| | | TGDSTYY | | | | | | |
| | | LDSVKGR | | | | | | |
| | | FTISRDNA | | | | | | |
| | | KNTVFLQ | | | | | | |
| | | MNSLKPE | | | | | | |
| | | DTAVYYC | | | | | | |
| | | AARGGGS | | | | | | |
| | | SVDSEYD | | | | | | |
| | | VGEFEYD | | | | | | |
| | | YWGQGT | | | | | | |
| | | QVTVSSE | | | | | | |
| | | PKTPKGG | | | | | | |
| | | CGGGLEH | | | | | | |
| | | HHHHH | | | | | | |

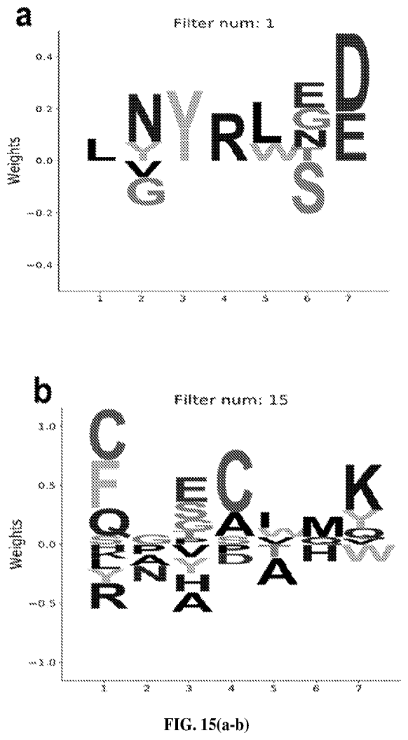

TABLE 4
GST summary: amino acid sequence filters
derived from a deep learning approach

| Region | | Activity in | Activity in |
|---|---|---|---|
| of | | Low affinity | High affinity |
| activity | Filter | prediction | prediction |
| Cdr3 | See FIG. 15A, | <1% | 56% (41% |
| | SEQ ID NO: | | in 5-best |
| | 2663 | | contributors) |
| Cdr3 | See FIG. 15B, | 76% (69% | <1% |
| | SEQ ID NO: | in 5-best | |
| | 2664 | contributors) | |

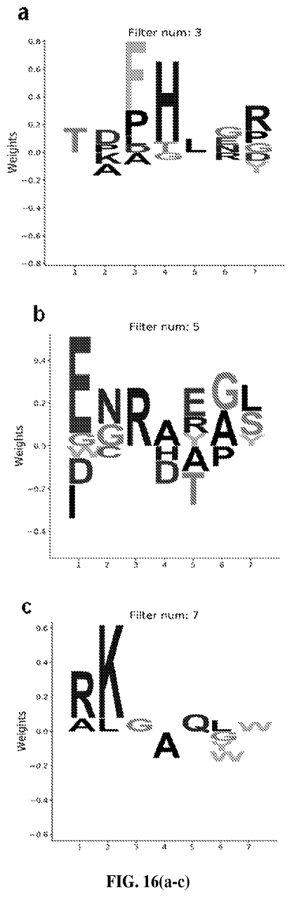

TABLE 5
HSA summary: amino acid sequence filters
derived from a deep learning approach

| Region | | Activity in | Activity in |
|---|---|---|---|
| of | | Low affinity | High affinity |
| activity | Filter | prediction | prediction |
| Cdr3 | See FIG. 16A, | 79% (65%% | 20% (<10% |
| | SEQ ID NO: | in 5-best | in 5-best |
| | 2665 | contributors) | contributors) |
| Cdr3 | See FIG. 16B; | <1% | 75%( 50% |
| | SEQ ID NO: | | in 5-best |
| | 2666 | | contributors) |
| | | | Most contributing |
| Cdr3 | See FIG. 16C; | <1% | 77% (27% |
| | SEQ ID NO: | | in 5-best |
| | 2667 | | contributors) |
| | | | Most activated |

### REFERENCES

1. Muyldermans, S. Nanobodies: natural single-domain antibodies. Annu Rev Biochem 82, 775-797 (2013).
2. Beghein, E. & Gettemans, J. Nanobody Technology: A Versatile Toolkit for Microscopic Imaging, Protein-Protein Interaction Analysis, and Protein Function Exploration. Front Immunol 8, 771 (2017).
3. Rasmussen, S. G. et al. Structure of a nanobody-stabilized active state of the beta(2) adrenoceptor. Nature 469, 175-180 (2011).
4. Jovcevska, I. & Muyldermans, S. The Therapeutic Potential of Nanobodies. BioDrugs 34, 11-26 (2020).
5. Lauwereys, M. et al. Potent enzyme inhibitors derived from dromedary heavy-chain antibodies. The EMBO journal 17, 3512-3520 (1998).
6. Pardon, E. et al. A general protocol for the generation of Nanobodies for structural biology. Nature protocols 9, 674-693 (2014).
7. McMahon, C. et al. Yeast surface display platform for rapid discovery of conformationally selective nanobodies. Nature structural & molecular biology 25, 289-296 (2018).
8. Egloff, P. et al. Engineered peptide barcodes for in-depth analyses of binding protein libraries. Nature methods 16, 421-428 (2019).
9. Fridy, P. C. et al. A robust pipeline for rapid production of versatile nanobody repertoires. Nature methods 11, 1253-1260 (2014).
10. Savitski, M. M., Wilhelm, M., Hahne, H., Kuster, B. & Bantscheff, M. A Scalable Approach for Protein False Discovery Rate Estimation in Large Proteomic Data Sets. Molecular & cellular proteomics: MCP 14, 2394-2404 (2015).
11. DeKosky, B. J. et al. High-throughput sequencing of the paired human immunoglobulin heavy and light chain repertoire. Nature biotechnology 31, 166-169 (2013).
12. Elias, J. E. & Gygi, S. P. Target-decoy search strategy for increased confidence in large-scale protein identifications by mass spectrometry. Nature methods 4, 207-214 (2007).
13. Schneidman-Duhovny, D., Inbar, Y., Nussinov, R. & Wolfson, HJ. PatchDock and SymmDock: servers for rigid and symmetric docking. Nucleic acids research 33, W363-W367 (2005).
14. Chait, B. T., Cadene, M., Olinares, P. D., Rout, M. P. & Shi, Y. Revealing Higher Order Protein Structure Using Mass Spectrometry. Journal of the American Society for Mass Spectrometry 27, 952-965 (2016).
15. Rout, M. P. & Sali, A. Principles for Integrative Structural Biology Studies. Cell 177, 1384-1403 (2019).
16. Yu, C. & Huang, L. Cross-Linking Mass Spectrometry: An Emerging Technology for Interactomics and Structural Biology. Analytical Chemistry 90, 144-165 (2018).
17. Leitner, A., Faini, M., Stengel, F. & Aebersold, R. Crosslinking and Mass Spectrometry: An Integrated Technology to Understand the Structure and Function of Molecular Machines. Trends in biochemical sciences 41, 20-32 (2016).
18. Larsen, M. T., Kuhlmann, M., Hvam, M. L. & Howard, K. A. Albumin-based drug delivery: harnessing nature to cure disease. Mol Cell Ther 4, 3 (2016).
19. Zhu, W. H., Smith, J. W. & Huang, C. M. Mass Spectrometry-Based Label-Free Quantitative Proteomics. J Biomed Biotechnol (2010).
20. Cox, J. & Mann, M. MaxQuant enables high peptide identification rates, individualized p.p.b.-range mass accuracies and proteome-wide protein quantification. Nature biotechnology 26, 1367-1372 (2008).
21. Shi, Y. et al. Structural characterization by cross-linking reveals the detailed architecture of a coatomer-related heptameric module from the nuclear pore complex. Molecular & cellular proteomics: MCP 13, 2927-2943 (2014).
22. Kim, S. J. et al. Integrative structure and functional anatomy of a nuclear pore complex. Nature 555, 475-482 (2018).
23. Pires, D. E. V., Ascher, D. B. & Blundell, T. L. mCSM: predicting the effects of mutations in proteins using graph-based signatures. Bioinformatics (Oxford, England) 30, 335-342 (2014).
24. Finn, J. A. et al. Improving Loop Modeling of the Antibody Complementarity-Determining Region 3 Using Knowledge-Based Restraints. PloS one 11, e0154811 (2016).
25. Tiller, K. E. et al. Arginine mutations in antibody complementarity-determining regions display context-dependent affinity/specificity trade-offs. The Journal of biological chemistry 292, 16638-16652 (2017).
26. Mitchell, L. S. & Colwell, L. J. Analysis of nanobody paratopes reveals greater diversity than classical antibodies. Protein Eng Des Sel 31, 267-275 (2018).
27. Desmyter, A. et al. Crystal structure of a camel single-domain VH antibody fragment in complex with lysozyme. Nat Struct Biol 3, 803-811 (1996).
28. Li, T. et al. Immuno-targeting the multifunctional CD38 using nanobody. Scientific reports 6 (2016).
29. Sheng, M. & Sala, C. PDZ domains and the organization of supramolecular complexes. Annu Rev Neurosci 24, 1-29 (2001).
30. Doyle, D. A. et al. Crystal structures of a complexed and peptide-free membrane protein-binding domain: Molecular basis of peptide recognition by PDZ. Cell 85, 1067-1076 (1996).
31. Niethammer, M. et al. CRIPT, a novel postsynaptic protein that binds to the third PDZ domain of PSD-95/SAP90. Neuron 20,693-707 (1998).
32. Akram, A. & Inman, R. D. Immunodominance: A pivotal principle in host response to viral infections. Clin Immunol 143, 99-115 (2012).
33. Bar-On, Y. M., Phillips, R. & Milo, R. The biomass distribution on Earth. Proceedings of the National Academy of Sciences of the United States of America 115, 6506-6511 (2018).
34. Chaplin, D. D. Overview of the immune response. J Allergy Clin Immun 125, S3-S23 (2010).
35. Acharya, P. et al. Heavy chain-only IgG2b llama antibody effects near-pan HIV-1 neutralization by recognizing a CD4-induced epitope that includes elements of coreceptor- and CD4-binding sites. J Virol 87, 10173-10181 (2013).
36. Arabi, Y. M. et al. Middle East Respiratory Syndrome. New EngI J Med 376,584-594 (2017).
37. Flajnik, M. F., Deschacht, N. & Muyldermans, S. A Case Of Convergence: Why Did a Simple Alternative to Canonical Antibodies Arise in Sharks and Camels? PLoS biology 9 (2011).
38. Sircar, A., Sanni, K. A., Shi, J. & Gray, J. J. Analysis and modeling of the variable region of camelid single-domain antibodies. J Immunol 186, 6357-6367 (2011).
39. Baran, D. et al. Principles for computational design of binding antibodies. Proceedings of the National Academy of Sciences of the United States of America 114, 10900-10905 (2017).
40. Chevalier, A. et al. Massively parallel de novo protein design for targeted therapeutics. Nature 550, 74-79 (2017).
41. Arbabi Ghahroudi, M., Desmyter, A., Wyns, L., Hamers, R. & Muyldermans, S. Selection and identification of single domain antibody fragments from camel heavy-chain antibodies. FEBS letters 414, 521-526 (1997).
42. Shi, Y. et al. A strategy for dissecting the architectures of native macromolecular assemblies. Nature methods 12, 1135-1138 (2015).
43. Chen, Z. L. et al. A high-speed search engine pLink 2 with systematic evaluation for proteome-scale identification of cross-linked peptides. Nature communications 10, 3404 (2019).
44. Dunbar, J. & Deane, C. M. ANARCI: antigen receptor numbering and receptor classification. Bioinformatics (Oxford, England) 32, 298-300 (2016).
45. Lefranc, M. P. et al. IMGT unique numbering for immunoglobulin and T cell receptor variable domains and Ig superfamily V-like domains. Dev Comp Immunol 27, 55-77 (2003).
46. Crooks, G. E., Hon, G., Chandonia, J. M. & Brenner, S. E. WebLogo: a sequence logo generator. Genome research 14, 1188-1190 (2004).
47. Sievers, F. & Higgins, D. G. Clustal Omega, accurate alignment of very large numbers of sequences. Methods in molecular biology 1079, 105-116 (2014).
48. Letunic, I. & Bork, P. Interactive Tree Of Life (iTOL): an online tool for phylogenetic tree display and annotation. Bioinformatics (Oxford, England) 23, 127-128 (2007).
49. Waterhouse, A. M., Procter, J. B., Martin, D. M., Clamp, M. & Barton, GJ. Jalview Version 2—a multiple sequence alignment editor and analysis workbench. Bioinformatics (Oxford, England) 25, 1189-1191 (2009).
50. Kall, L., Canterbury, J. D., Weston, J., Noble, W. S. & MacCoss, M. J. Semi-supervised learning for peptide identification from shotgun proteomics datasets. Nature methods 4, 923-925 (2007).
51. Webb, B. & Sali, A. Comparative Protein Structure Modeling Using MODELLER. Curr Protoc Bioinformatics 47, 5 6 1-32 (2014).
52. Dong, G. Q., Fan, H., Schneidman-Duhovny, D., Webb, B. & Sali, A. Optimized atomic statistical potentials: assessment of protein interfaces and loops. Bioinformatics (Oxford, England) 29, 3158-3166 (2013).
53. Schneidman-Duhovny, D. & Wolfson, H. J. Modeling of Multimolecular Complexes. Methods in molecular biology 2112, 163-174 (2020).
54. Russel, D. et al. Putting the pieces together: integrative modeling platform software for structure determination of macromolecular assemblies. PLoS biology 10, e1001244 (2012).
55. Fernandez-Martinez, J. et al. Structure and Function of the Nuclear Pore Complex Cytoplasmic mRNA Export Platform. Cell 167, 1215-1228 e1225 (2016).

1. A method of identifying a group of complementarity determining region (CDR)3, 2 and/or 1 nanobody amino acid sequences (CDR3, CDR2 and/or CDR1 sequences) wherein a reduced number of the CDR3, CDR2 and/or CDR1 sequences are false positives as compared to a control, the method comprising:
a. obtaining a blood sample from a camelid immunized with an antigen;
b. using the blood sample to obtain a nanobody cDNA library;
c. identifying the sequence of each cDNA in the library;
d. isolating nanobodies from the same or a second blood sample from the camelid immunized with the antigen;
e. digesting the nanobodies with trypsin or chymotrypsin to create a group of digestion products;
f. performing a mass spectrometry analysis of the digestion products to obtain mass spectrometry data;
g. selecting sequences identified in step c. that correlate with the mass spectrometry data;
h. identifying sequences of CDR3, CDR2 and/or CDR1 regions in the sequences from step g.; and
i. selecting from the CDR3, CDR2 and/or CDR1 region sequences of step h. those sequences having equal to or more than a required fragmentation coverage percentage; wherein the fragmentation coverage percentage is determined by a formula f(x,chymotrypsin)=0.0023x²−0.0497x+0.7723,x[5,30] when chymotrypsin is used in step e. or a formula f(x,trypsin)=0.00006x²−0.00444x+0.9194, x[5,30] when trypsin is used in step e., and wherein x is the length of the CDR3, CDR2 or CDR1 region sequence, respectively; and
j. wherein the selected sequences of step i. comprise a group having the reduced number of false positive CDR3, CDR2 and/or CDR1 sequences.

2. The method of claim 1, wherein the required fragmentation coverage percentage is about 30.

3. The method of claim 1, wherein the required fragmentation coverage percentage is about 50 and trypsin is used in step e.

4. The method of claim 1, wherein the required fragmentation coverage percentage is about 40 and chymotrypsin is used in step e.

5. The method of claim 1, wherein step d. comprises obtaining plasma from the blood sample and isolating nanobodies using one or more affinity isolation methods.

6. The method of claim 5, wherein the one or more affinity isolation methods of step d. comprise one or more of protein G sepharose affinity chromatography and protein A sepharose affinity chromatography.

7. The method of claim 1, wherein step d. further comprises a functional selection step comprising selecting antigen-specific nanobodies using an antigen-specific affinity chromatography and eluting the antigen-specific nanobodies under varying degrees of stringency thereby creating different nanobody fractions, and performing steps e. through i. on each fraction individually and estimating an affinity of each different step i. CDR3, CDR2 and/or CDR1 region sequence for the antigen based on a relative abundance of the CDR3, CDR2 and/or CDR1 region sequence in each of the nanobody fractions, respectively.

8. The method of claim 7, wherein the antigen-specific affinity chromatography is a resin conjugated to the antigen.

9. The method of claim 7, wherein the antigen-specific affinity chromatography is a resin coupled to maltose binding protein and the antigen.

10. The method of claim 1, further comprising creating a CDR3, CDR2 and/or CDR1 peptide having a sequence identified in step i.

11. The method of claim 1, further comprising creating a nanobody comprising a CDR3, CDR2 and/or CDR1 region having a sequence identified in step i.

12. A nanobody comprising an amino acid sequence selected from SEQ ID NOs: 1-2536 and SEQ ID NOs: 2665-2667.

13. A computer-implemented method, comprising:
receiving a nanobody peptide sequence;
identifying a plurality of complementarity-determining region (CDR) regions of the nanobody peptide sequence, the CDR regions including CDR3, CDR2 and/or CDR1 regions;
applying a fragmentation filter to discard one or more false positive CDR3, CDR2 and/or CDR1 regions of the nanobody peptide sequence;
quantifying an abundance of one or more non-discarded CDR3, CDR2 and/or CDR1 regions of the nanobody peptide sequence; and
inferring an antigen affinity based on the quantified abundance of the one or more non-discarded CDR3, CDR2 and/or CDR1 regions of the nanobody peptide sequence.

14. The computer-implemented method of claim 13, further comprising classifying the one or more non-discarded CDR3, CDR2 and/or CDR1 regions of the nanobody peptide sequence as having a low antigen affinity, mediocre antigen affinity, or high antigen affinity.

15. The method of claim 14, further comprising assembling the one or more non-discarded CDR3, CDR2 and/or CDR1 regions of the nanobody peptide sequence classified as having the high antigen affinity into a nanobody protein.

16. The computer-implemented method of claim 13, wherein the fragmentation filter is configured to require a minimum calculated fragmentation coverage percentage.

17. The computer-implemented method of claim 16, wherein the minimum calculated fragmentation coverage percentage is about 30.

18. The computer-implemented method of claim 17, wherein the minimum calculated fragmentation coverage percentage is about 50 for trypsin-treated samples and about 40 for chymotrypsin-treated samples.

19. The computer-implemented method of claim 13, further comprising:
receiving a plurality of nanobody peptide sequences; and
comparing each of the nanobody peptide sequences to a database to separate the nanobody peptide sequences into an excluded subgroup and a non-excluded subgroup, wherein the nanobody peptide sequences of the excluded subgroup are not found in the database, and wherein the CDR regions are only identified in the nanobody peptide sequences of the non-excluded subgroup.

20. The computer-implemented method of claim 13, wherein the abundance of one or more non-discarded CDR3, CDR2 and/or CDR1 regions of the nanobody peptide sequence is quantified based on relative MS1 ion signal intensities.

21. The computer-implemented method of claim 13, wherein the antigen affinity is inferred using k-means clustering based on epitope similarity.

22. A method for training a deep learning model, comprising:
creating a dataset using the computer-implemented method of claim 13; and
training, using the dataset, a deep learning model to classify nanobody peptide sequences having low antigen affinity and nanobody peptide sequences having high antigen affinity, wherein the dataset comprises a plurality of nanobody peptide sequences and corresponding antigen-affinity labels.

23. The method of claim 22, wherein the deep learning model is a convolutional neural network.

24. A method for determining antigen affinity of nanobody peptide sequences, comprising:
receiving a nanobody peptide sequence;
inputting the nanobody peptide sequence into a trained deep learning model; and
classifying, using the trained deep learning model, the nanobody peptide sequence as having low antigen affinity or high antigen affinity.

25. The method of claim 24, wherein the deep learning model is a convolutional neural network.

26. The method of claim 24, wherein the trained deep learning model is trained.
